(19) United States
(12) Patent Application Publication
(10) Pub. No.: US 2022/0112204 A1
(43) Pub. Date: Apr. 14, 2022

(54) SUBSTITUTED DIHYDROPYRANOPYRIMIDINE COMPOUNDS AS KRAS INHIBITORS

(71) Applicant: ACCUTAR BIOTECHNOLOGY INC., Brooklyn, NY (US)

(72) Inventors: Jie FAN, New York, NY (US); Yimin QIAN, Plainsboro, NJ (US); Wei HE, Zionsville, IN (US); Robert Z. LUO, New City, NY (US)

(73) Assignee: ACCUTAR BIOTECHNOLOGY INC., Brooklyn, NY (US)

(21) Appl. No.: 17/500,440

(22) Filed: Oct. 13, 2021

Related U.S. Application Data
(60) Provisional application No. 63/091,393, filed on Oct. 14, 2020.

(51) Int. Cl. C07D 491/052; A61K 31/519; A61P 35/00

(52) U.S. Cl. CPC C07D 491/052; A61P 35/00; A61K 31/519

(57) ABSTRACT

The present disclosure relates to novel compounds that inhibit KRAS G120, pharmaceutical compositions containing such compounds, and their use in prevention and treatment of cancer and related diseases and conditions.

This application claims priority from U.S. Provisional Patent Application No. 63/091,393, filed Oct. 14, 2020, which is hereby incorporated by reference in its entirety.

### FIELD OF THE DISCLOSURE

The present invention relates to compounds that inhibit KRas G12C. In particular, the present invention relates to compounds that irreversibly inhibit the activity of KRas G12C, pharmaceutical compositions comprising the compounds and methods of use therefor.

### BACKGROUND OF THE DISCLOSURE

The KRAS, NRAS and HRAS genes encode a set of closely related small GTPase proteins KRas, NRas and HRas, collectively referred to herein as the Ras proteins or Ras, that share 82-90% overall sequence identity. The Ras proteins are critical components of signalling pathways transmitting signals from cell-surface receptors to regulate cellular proliferation, survival and differentiation. Ras functions as a molecular switch cycling between an inactive GDP-bound state and an active GTP-bound state. The GDP/GTP cycle of Ras is tightly regulated in cells by guanine nucleotide exchange factors (GEFs) such as Sos1 and Sos2, which promote the exchange of GDP for GTP, and GTPase activating proteins (GAPs) such as NF-1 and p120RasGAP which stimulate the intrinsic GTPase activity of Ras hydrolysing GTP to GDP.

The Ras proteins are 188-189 amino acids in length and have a highly conserved N-terminal G-domain containing the p-loop region, which binds nucleotide, and the switch I and switch II regions which are important for regulatory and effector protein interactions. The C-terminal region of the Ras proteins are more divergent and contain elements which regulate the association of Ras with the membrane including the conserved carboxyl terminal CAXX box motif which is necessary for post-translational prenylation modifications. On binding to GTP the switch I and switch II regions of Ras undergo a conformational change which enables its interaction and activation of effector proteins to regulate down-stream signalling pathways. The best characterized effector of Ras is the serine/threonine kinase Raf which regulates the activity of the mitogen-activate protein kinase (MAPK) pathway. The PI3K pathway is another important effector pathway down-stream of Ras with the p110 catalytic subunit of the class I phosphoinositide 3-kinases interacting with Ras. Other effectors of Ras including RaIGDS, Tiaml, PLC-ε and Rassfl have also been described (See, Cox et al. Nature Reviews Drug Discovery, 13:828-851 (2014)).

RAS mutations are frequently found in cancer and approximately 30% of all human cancers have a mutation in KRAS, NRAS or HRAS genes. Oncogenic Ras is typically, but not exclusively, associated with mutations at glycine 12, glycine 13 or glutamine 61 of Ras. These residues are located at the active site of Ras and mutations impair intrinsic and/or GAP-catalyzed GTPase activity favouring the formation of GTP bound Ras and aberrant activation of down-stream effector pathways. KRAS is the most frequently mutated RAS gene in cancer followed by NRAS and then HRAS.

Glycine to cysteine mutation at residue 12 of Ras (the G12C mutation) is generated from a G.C to T.A base transversion at codon 12, a mutation commonly found in RAS genes that accounts for 14% of all KRAS, 2% of all NRAS and 2% of all HRAS mutations across cancer types. The G12C mutation is particularly enriched in KRAS mutant non-small cell lung cancer with approximately half carrying this mutation, which has been associated with the DNA adducts formed by tobacco smoke. The G12C mutation is not exclusively associated with lung cancer and is found in other RAS mutant cancer types including 8% of all KRAS mutant colorectal cancer.

There are several tumour types that exhibit a high frequency of activating mutations in KRAS including pancreatic (˜90% prevalence), colorectal (˜40% prevalence) and non-small cell lung cancer (˜30% prevalence). KRAS mutations are also found in other cancer types including multiple myeloma, uterine cancer, bile duct cancer, stomach cancer, bladder cancer, diffuse large B cell lymphoma, rhabdomyosarcoma, cutaneous squamous cell carcinoma, cervical cancer, testicular germ cell cancer and others.

There remains an unmet medical need for new medical treatments for patients with pancreatic cancer, lung adenocarcinoma, or colorectal cancer, especially those who have been diagnosed to have such cancers characterized by a KRAS mutation, and including those having cancer that progressed after chemotherapy.

### SUMMARY OF THE DISCLOSURE

In some embodiments, the present disclosure is directed to a compound of Formula (1) or a tautomer, stereoisomer or a mixture of stereoisomers, or a pharmaceutically acceptable salt, or hydrate, or deuterated derivative thereof:

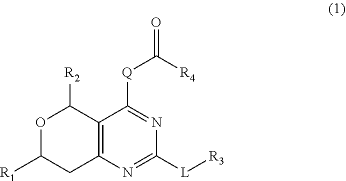

wherein:
R₁ is chosen from an aryl group, cycloalkyl, heterocycle, and heteroaryl, wherein each of the aryl group, cycloalkyl, heterocycle, and heteroaryl is independently substituted with 0, 1, 2, 3, or 4 R₅;
R₂ is chosen from hydrogen, C₁-C₄ alkyl, and cycloalkyl;
R₃ is chosen from hydrogen, C₁-C₆ alkyl, C₁-C₆ alkoxyalkyl, C₁-C₆ hydroxyalkyl, C₁-C₆ dihydroxyalkyl, C₁-C₆ dialkylaminoalkyl, cycloalkyl, heterocycle, heterocycloalkyl, aryl, and heteroaryl, each of which is independently substituted with 0, 1, 2, 3, or 4 R₆;
R₄ is chosen from a C₂-C₆ alkenyl and C₂-C₆ alkynyl, wherein each of the C₂-C₆ alkenyl and C₂-C₆ alkynyl is independently substituted with hydrogen, halogen, —CN, alkyl, haloalkyl, alkoxyalkyl, dialkylaminoalkyl, and heterocyclic alkyl;
R₅ is chosen from halogen, C₁-C₆ alkyl, C₂-alkynyl, C₁-C₃haloalkyl, deuterated C₁-C₆ alkyl, dialkylamino group, amino group, —CN, hydroxyl, C₁-₄alkoxy, cycloalkyl, and heterocycle;
R₆ is chosen from halogen, C₁-C₆ alkyl, C₁-C₃haloalkyl, dialkylamino group, amino group, —CN, hydroxyl, C₁-C₄alkoxy, cycloalkyl, heterocycle, heterocycloalkyl, and heteroaryl;
L is a bond, O, S, NH, or alkylamino;
Q is a 4-12 membered heteromonocyclic group, bridged or spiro heterocyclic group, wherein each of the heteromonocyclic group, bridged or spiro heterocyclic group is independently substituted with 0, 1, 2, 3, or 4 R₇;
R₇ is a C₁-C₄ alkyl, wherein the C₁-C₄ alkyl is further substituted with hydrogen, halogen, —CN, ester, and carboxamide.

In some embodiments, the compound of Formula (1) may be a compound of Formula (1A)

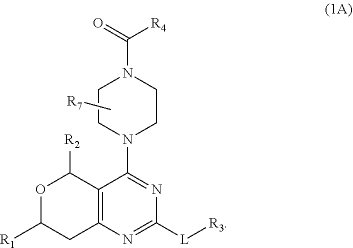

In some embodiments, the compound of Formula (1) may be a compound of Formula (1B)

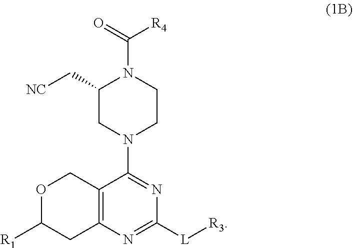

In some embodiments, the compound of Formula (1) may be a compound of Formula (1C)

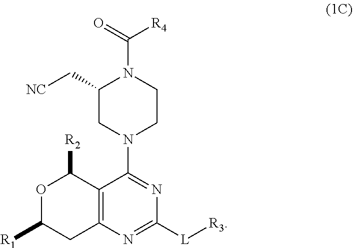

In some embodiments, R₁ is chosen from phenyl, naphthyl, pyridyl, indazolyl, indolyl, indanyl, azaindolyl, indolinyl, benzotriazolyl, benzoxadiazolyl, cinnolinyl, imidazopyridinyl, pyrazolopyridinyl, quinolinyl, isoquinolinyl, quinazolinyl, quinazolinonyl, indolinonyl, isoindolinonyl, tetrahydronaphthyl, tetrahydroquinolinyl, and tetrahydroisoquinolinyl, each of which is substituted with 0, 1, 2, 3, or 4 R₅.

In some embodiments, R₁ is chosen from

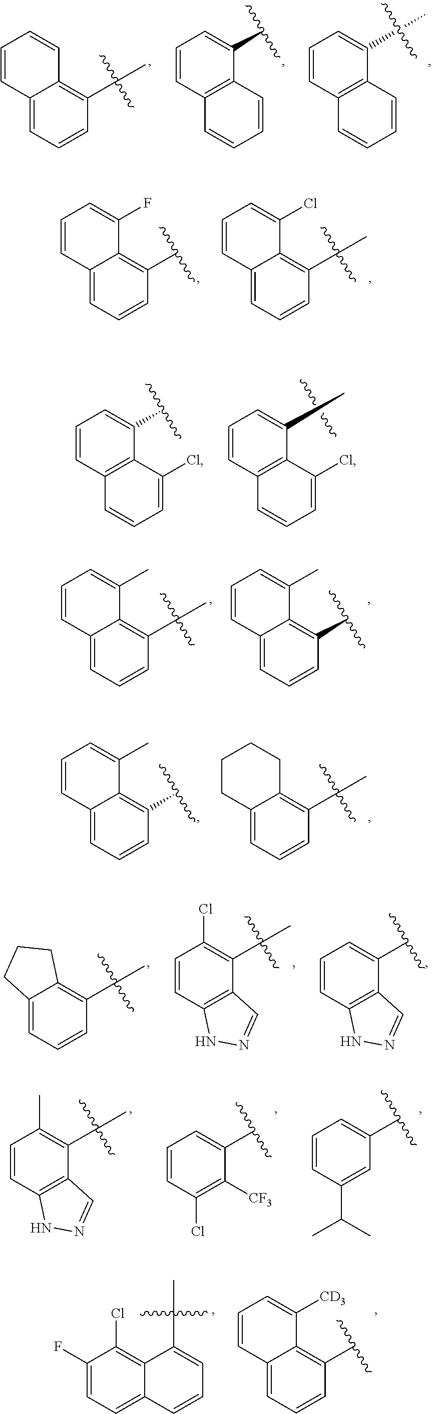

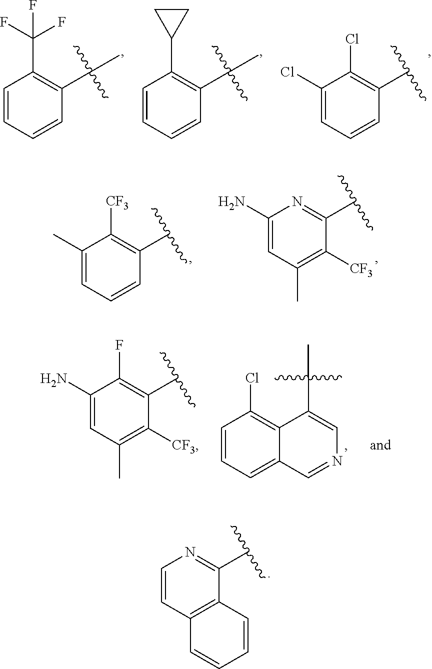

In some embodiments, R₅ is chosen from halogen, C₂-alkynyl, C₁-C₄ alkyl, CF₃, deuterated C₁-C₄ alkyl, amino group, —CN, hydroxyl, C₁-C₄alkoxy, and cycloalkyl. In some embodiments, R₅ is chosen from halogen, C₂-alkynyl, C₁-C₃ alkyl, C₁-C₃haloalkyl, deuterated C₁-C₃ alkyl, amino group, and —CN.

In some embodiments, R₂ is chosen from hydrogen, C₁-C₃alkyl, and cycloalkyl.

In some embodiments, R₃ is chosen from

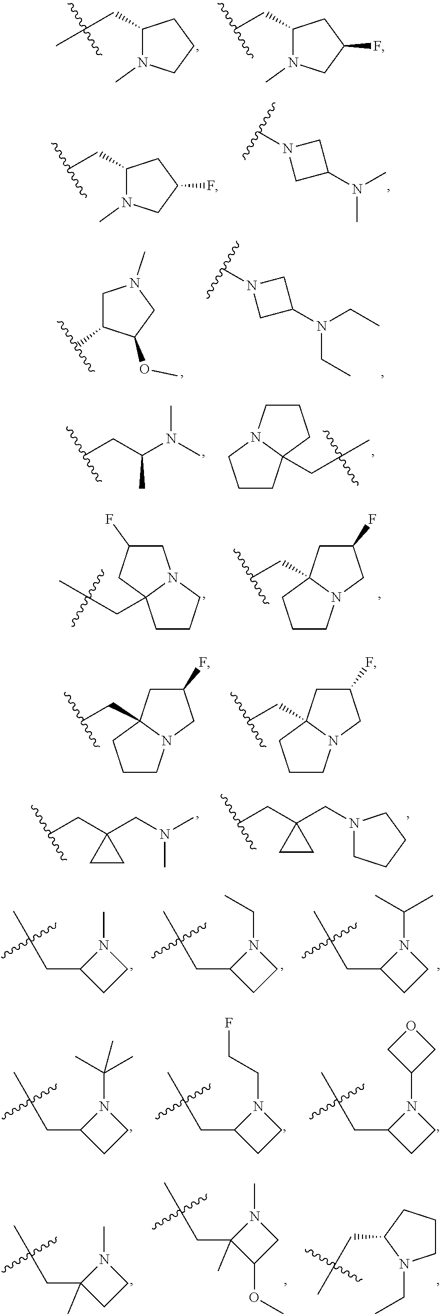

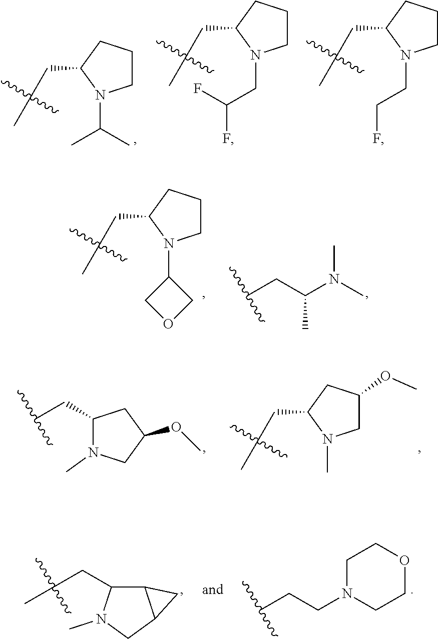

In some embodiments, R₆ is chosen from halogen, C₁-C₃ alkyl, C₁-C₃haloalkyl, C₁-C₄alkoxy, dialkylamino group, cycloalkyl, heterocycle, heterocycloalkyl, and heteroaryl.

In some embodiments, R₄ is chosen from —CH═CH₂,

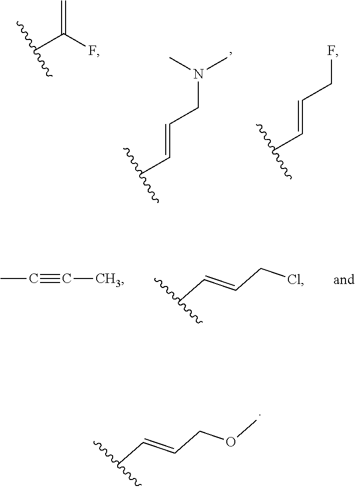

In some embodiments, L is a bond or O. In one embodiment, L is a bond. In one embodiment, L is O.

In some embodiments, Q is a 4-12 membered heteromonocyclic group, bridged or spiro heterocyclic group, wherein each of the heteromonocyclic group, bridged or spiro heterocyclic group is independently substituted with 0, 1, or 2 R₇.

In some embodiments, R₇ is a C₁-C₄ alkyl, wherein the C₁-C₄ alkyl is further substituted with hydrogen, halogen, —CN, ester, and carboxamide.

In some embodiments, Q is chosen from

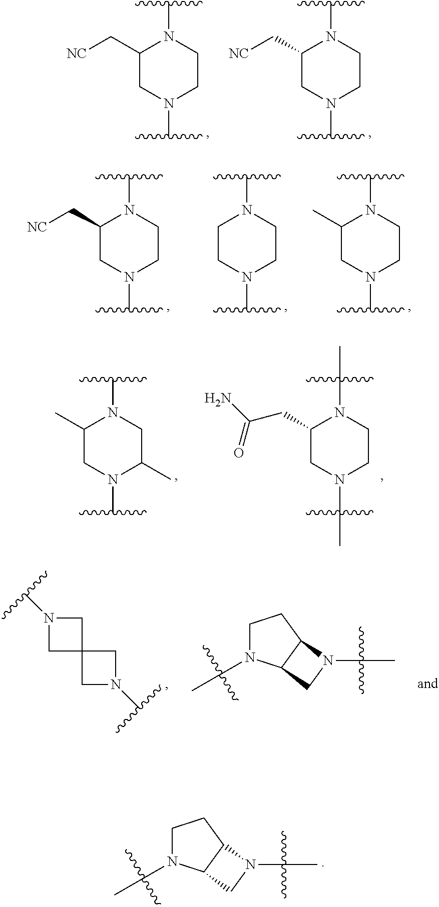

In some embodiments, the compound of Formula (1) (e.g. Formula (1A), Formula (1B), Formula (1C)) may encompass both stereoisomes and a mixture of stereoisomers. In some embodiments, the compound of Formula (1) may encompass both racemic isomers and enantiomeric isomers.

Also disclosed herein is a method of treating cancer, in a subject in need thereof, comprising administering to said subject a compound of Formula (1) (e.g. Formula (1A), Formula (1 B), Formula (1C)) or pharmaceutically acceptable salt thereof, or a pharmaceutical composition comprising a compound of Formula (1) or a pharmaceutically acceptable salt thereof. In at least one embodiment, the pharmaceutical composition of the present disclosure may be for use in (or in the manufacture of medicaments for) the treatment of cancer in the subject in need thereof.

In at least one embodiment, a therapeutically-effective amount of a pharmaceutical composition of the present disclosure may be administered to a subject diagnosed with cancer. In some embodiments, the cancer is chosen from breast cancer, lung cancer, pancreatic cancer, colorectal cancer, gall bladder cancer, thyroid cancer, bile duct cancer, ovarian cancer, endometrial cancer, prostate cancer, and esophageal cancer.

### BRIEF DESCRIPTION OF THE FIGURES

The accompanying drawings, which are incorporated in and constitute a part of this specification, illustrate the disclosed embodiments and, together with the description, explain the principles of the disclosed embodiments. In the drawings:

FIG. 1 illustrates mobility change of KRAS-G12C mutant or wild type proteins on SDS-PAGE after 5 minutes of coincubation at 25° C. with exemplary compounds of the present disclosure, indicative of covalent cysteine conjugation.

FIG. 2 illustrates phospho-ERK1/2 (Thr202/Tyr204) inhibition by exemplary compounds 4, 18, 55 and 64 of the present disclosure in a MIA PaCa-2 cell line 4 hours after incubation.

### DETAILED DESCRIPTION OF THE DISCLOSURE

### Definitions

A dash (“—”) that is not between two letters or symbols is used to indicate a point of attachment for a substituent. For example, —CN is attached through the carbon atom.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “acyl” as used herein refers to R—C(O)— groups such as, but not limited to, (alkyl)-C(O)—, (alkenyl)-C(O)—, (alkynyl)-C(O)—, (aryl)-C(O)—, (cycloalkyl)-C(O)—, (heteroaryl)-C(O)—, and (heterocyclyl)-C(O)—, wherein the group is attached to the parent molecular structure through the carbonyl functionality. In some embodiments, it is a C₁₋₁₀ acyl radical which refers to the total number of chain or ring atoms of the, for example, alkyl, alkenyl, alkynyl, aryl, cycloalkyl, or heteroaryl, portion plus the carbonyl carbon of acyl. For example, a C₄-acyl has three other ring or chain atoms plus carbonyl.

The term “alkenyl” as used herein refers to an unsaturated straight or branched hydrocarbon having at least one carbon-carbon double bond, such as a straight or branched group of 2-8 carbon atoms, referred to herein as (C₂₋C₈)alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkyl” as used herein refers to a saturated straight or branched hydrocarbon, such as a straight or branched group of 1-8 carbon atoms, referred to herein as C₁₋₈ alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

The term “alkylene” as used herein referes to a divalent alkyl radical. Representative examples of C₁₋₁₀ alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene.

The term “alkynyl” as used herein refers to an unsaturated straight or branched hydrocarbon having at least one carbon-carbon triple bond, such as a straight or branched group of 2-8 carbon atoms, referred to herein as (C₂-C₈)alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “aryl” as used herein refers to a mono-, bi-, or other multi-carbocyclic, aromatic ring system with 5 to 14 ring atoms. The aryl group can optionally be fused to one or more rings selected from aryls, cycloalkyls, heteroaryls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms.

The term “cyano” as used herein refers to —CN.

The term “cycloalkyl” as used herein refers to a saturated or unsaturated cyclic, bicyclic, or bridged bicyclic hydrocarbon group of 3-16 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —Cl, —Br, and/or —I.

“Haloalkyl” means an alkyl group substituted with one or more halogens. Examples of haloalkyl groups include, but are not limited to, trifluoromethyl, difluoromethyl, pentafluoroethyl, trichloromethyl, etc.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Exemplary heteroaryl groups include, but are not limited to, a monocyclic aromatic ring, wherein the ring comprises 2-5 carbon atoms and 1-3 heteroatoms, referred to herein as “(C₂-C₅)heteroaryl.” Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups also include, but are not limited to, a bicyclic aromatic ring, wherein the ring comprises 5-14 carbon atoms and 1-3 heteroatoms, referred to herein as “(C₅-C₁₄)heteroaryl.” Representative examples of heteroaryl include, but not limited to, indazolyl, indolyl, azaindolyl, indolinyl, benzotriazolyl, benzoxadiazolyl, imidazolyl, cinnolinyl, imidazopyridyl, pyrazolopyridyl, pyrrolopyridyl, quinolinyl, isoquinolinyl, quinazolinyl, quinazolinonyl, indolinonyl, isoindolinonyl, tetrahydronaphthyl, tetrahydroquinolinyl, tetrahydroisoquinolinyl, and the like.

The terms “heterocycle,” “heterocyclyl,” or “heterocyclic” as used herein each refer to a saturated or unsaturated 3- to 18-membered ring containing one, two, three, or four heteroatoms independently selected from nitrogen, oxygen, phosphorus, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

“Spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. A (C₃₋₁₂)spirocycloalkyl is a spirocycle containing between 3 and 12 carbon atoms.

“Spiroheterocycloalkyl” or “spiroheterocyclyl” means a spirocycle wherein at least one of the rings is a heterocycle one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

“Isomers” means compounds having the same number and kind of atoms, and hence the same molecular weight, but differing with respect to the arrangement or configuration of the atoms in space.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

It is well-known in the art that the biological and pharmacological activity of a compound is sensitive to the stereochemistry of the compound. For example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

The compounds disclosed herein may exist as tautomers and both tautomeric forms are intended to be encompassed by the scope of the present disclosure, even if only one tautomeric structure is depicted.

Additionally, unless otherwise stated, structures described herein are also meant to include compounds that differ only in the presence of one or more isotopically enriched atoms. For example, compounds having the present structures except for the replacement of hydrogen by deuterium (²H) or tritium (³H), or the replacement of a carbon by a ¹³C- or ¹⁴C-carbon atom are within the scope of this disclosure. Such compounds may be useful as, for example, analytical tools, probes in biological assays, or therapeutic agents.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, “a pharmaceutically acceptable salt” and/or “deuterated derivative thereof” is intended to encompass pharmaceutically acceptable salts of any one of the referenced compounds, deuterated derivatives of any one of the referenced compounds, and pharmaceutically acceptable salts of those deuterated derivatives.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw® Professional, version 17.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

As used herein, “cancer” refers to diseases, disorders, and conditions that involve abnormal cell growth with the potential to invade or spread to other parts of the body. Exemplary cancers include, but are not limited to, breast cancer, lung cancer, ovarian cancer, endometrial cancer, prostate cancer, and esophageal cancer.

“Subject” refers to an animal, such as a mammal, that has been or will be the object of treatment, observation, or experiment. The methods described herein may be useful for both human therapy and veterinary applications. In one embodiment, the subject refers to, for example, primates (e.g., humans, male or female), cows, sheep, goats, horses, dogs, cats, rabbits, rats, mice, fish, birds and the like. In some embodiments, the subject is a primate. In some embodiments, the subject is a human.

As used herein, the term “inhibit,” “inhibition,” or “inhibiting” refers to the reduction or suppression of a given condition, symptom, or disorder, or disease, or a significant decrease in the baseline activity of a biological activity or process.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically (e.g., through stabilization of a discernible symptom), physiologically, (e.g., through stabilization of a physical parameter), or both. In yet another embodiment, “treat,” “treating,” or “treatment” refers to preventing or delaying the onset or development or progression of the disease or disorder.

As used herein, a subject is “in need of” a treatment if such subject would benefit biologically, medically or in quality of life from such treatment.

### Compounds

In some embodiments, provided herein are compounds of Formula (I), or a tautomer, stereoisomer or a mixture of stereoisomers, or a pharmaceutically acceptable salt, or hydrate, or deuterated derivative thereof:

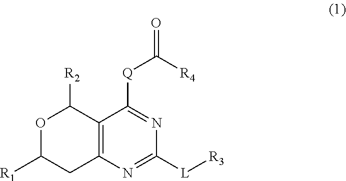

wherein:
R₁ is chosen from an aryl group, cycloalkyl, heterocycle, and heteroaryl, wherein each of the aryl group, cycloalkyl, heterocycle, and heteroaryl is independently substituted with 0, 1, 2, 3, or 4 R₅;
R₂ is chosen from hydrogen, C₁-C₄ alkyl, and cycloalkyl;
R₃ is chosen from hydrogen, C₁-C₆ alkyl, C₁-C₆ alkoxyalkyl, C₁-C₆ hydroxyalkyl, C₁-C₆ dihydroxyalkyl, C₁-C₆ dialkylaminoalkyl, cycloalkyl, heterocycle, heterocycloalkyl, aryl, and heteroaryl, each of which is independently substituted with 0, 1, 2, 3, or 4 R₆;
R₄ is chosen from a C₂-C₆ alkenyl and C₂-C₆ alkynyl, wherein each of the C₂-C₆ alkenyl and C₂-C₆ alkynyl is independently substituted with hydrogen, halogen, —CN, alkyl, haloalkyl, alkoxyalkyl, dialkylaminoalkyl, and heterocyclic alkyl;
R₅ is chosen from halogen, C₁-C₆ alkyl, C₂-alkynyl, C₁-C₃haloalkyl, deuterated C₁-C₆ alkyl, dialkylamino group, amino group, —CN, hydroxyl, C₁-C₄alkoxy, cycloalkyl, and heterocycle;
R₆ is chosen from halogen, C₁-C₆ alkyl, C₁-C₃haloalkyl, dialkylamino group, amino group, —CN, hydroxyl, C₁-C₄alkoxy, cycloalkyl, heterocycle, heterocycloalkyl, and heteroaryl;
L is a bond, O, S, NH, or alkylamino;
Q is a 4-12 membered heteromonocyclic group, bridged or spiro heterocyclic group, wherein each of the heteromonocyclic group, bridged or spiro heterocyclic group is independently substituted with 0, 1, 2, 3, or 4 R_{7;}
R₇ is a C₁-C₄alkyl, wherein the C₁-C₄ alkyl is further substituted with hydrogen, halogen, —CN, ester, and carboxamide.

In some embodiments, the compound of Formula (1) may be a compound of Formula (1A)

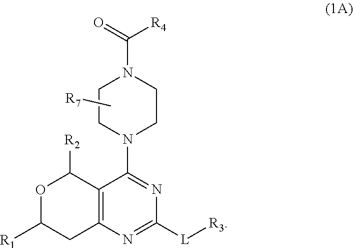

In some embodiments, the compound of Formula (1) may be a compound of Formula (1B)

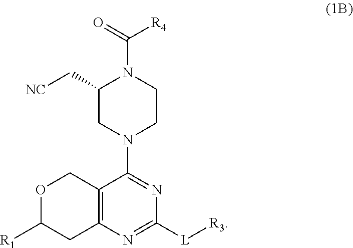

In some embodiments, the compound of Formula (1) may be a compound of Formula (1C)

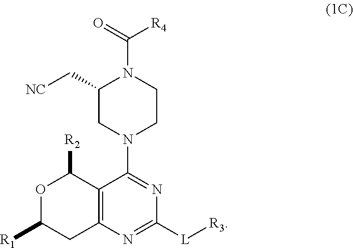

In some embodiments, the compound of Formula (1) is a compound of Formula (1A), Formula (1B), or Fomula (C), with the proviso that the compound of Formula (1) is not

2-((2S)-1-acryloyl-4-(2-(((S)-1-methylpyrrolidin-2-yl)methoxy)-7-(naphthalen-1-yl)-7,8-dihydro-5H-pyrano[4,3-c]pyrimidin-4yl)piperazin-2-yl)acetonitrile;

2-((2S)-1-acryloyl-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;

2-((S)-1-acryloyl-4-((S)-2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;

2-((S)-1-acryloyl-4-((R)-2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;

2-((S)-1-acryloyl-4-((R)-7-(8-chloronaphthalen-1-yl)-2-(((S)-1-methylpyrrolidin-2-yl)methoxy)-7,8-dihydro-5H-pyrano[4,3-c]pyrimidin-4-yl)piperazin-2-yl)acetonitrile;

2-((S)-1-acryloyl-4-((S)-7-(8-chloronaphthalen-1-yl)-2-(((S)-1-methylpyrrolidin-2-yl)methoxy)-7,8-dihydro-5H-pyrano[4,3-c]pyrimidin-4-yl)piperazin-2-yl)acetonitrile;

2-((2S)-1-acryloyl-4-(7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;

2-((S)-1-acryloyl-4-((S)-7-(8-methylnaphthalen-1-yl)-2-(((S)-1-methylpyrrolidin-2-yl)methoxy)-7,8-dihydro-5H-pyrano[4,3-c]pyrimidin-4-yl)piperazin-2-yl)acetonitrile;

2-((S)-1-acryloyl-4-((R)-7-(8-methylnaphthalen-1-yl)-2-(((S)-1-methylpyrrolidin-2-yl)methoxy)-7,8-dihydro-5H-pyrano[4,3-c]pyrimidin-4-yl)piperazin-2-yl)acetonitrile;

2-((2S)-1-(2-fluoroacryloyl)-4-(7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;

2-((S)-1-(2-fluoroacryloyl)-4-((S)-7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;

2-((S)-1-(2-fluoroacryloyl)-4-((R)-7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;

2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile;

2-((S)-4-((S)-7-(8-chloronaphthalen-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile;

2-((S)-4-((R)-7-(8-chloronaphthalen-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile;

2-((2S)-1-((E)-4-(dimethylamino)but-2-enoyl)-4-(7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile; or 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-1-((E)-4-(dimethylamino)but-2-enoyl)piperazin-2-yl)acetonitrile.

In some embodiments, R₁ is selected from an aromatic or heteroaromatic ring. In some embodiments, the aromatic or heteroaromatic ring is monocyclic or bicyclic. In some embodiments, the aromatic or heteroaromatic ring is each substituted with halogen, C₁-C₄ alkyl, CF₃, amino, CN, OH, and C₁-C₄ alkoxyl.

In some embodiments, R₁ is chosen from phenyl, naphthyl, pyridyl, indazolyl, indolyl, indanyl, azaindolyl, indolinyl, benzotriazolyl, benzoxadiazolyl, cinnolinyl, imidazopyridinyl, pyrazolopyridinyl, quinolinyl, isoquinolinyl, quinazolinyl, quinazolinonyl, indolinonyl, isoindolinonyl, tetrahydronaphthyl, tetrahydroquinolinyl, and tetrahydroisoquinolinyl, each of which is substituted with 0, 1, 2, 3, or 4 R₅;

In some embodiments, R₁ is chosen from

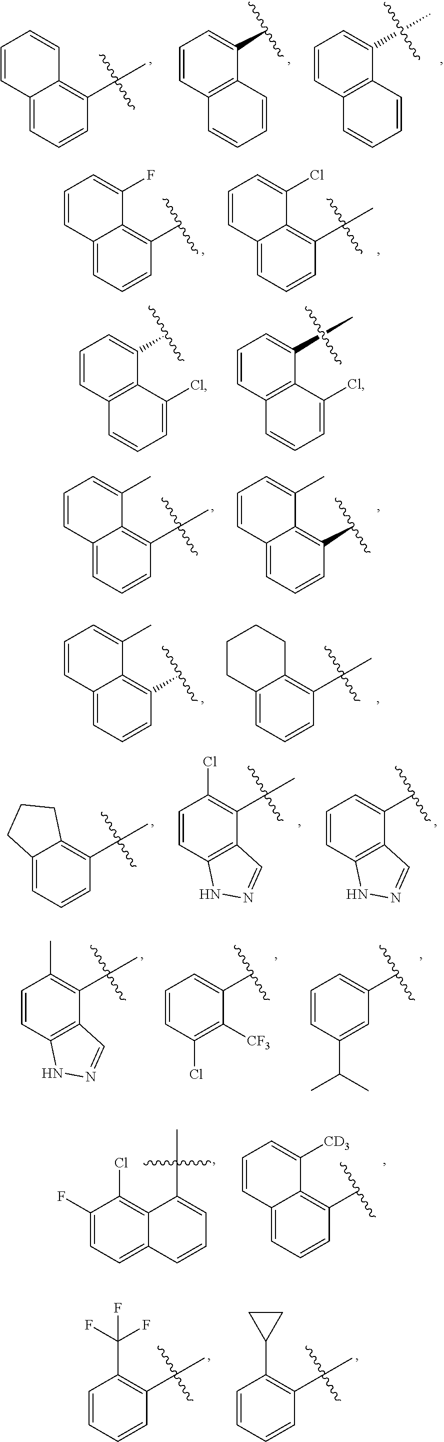

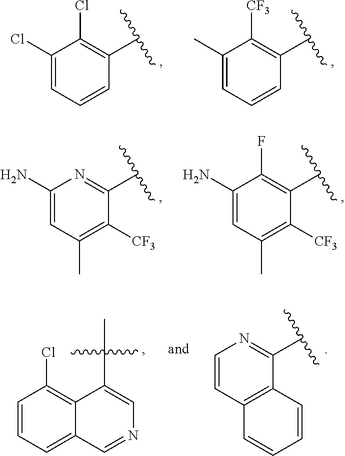

In some embodiments, R₁ is

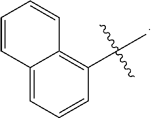 [SMILES: *c1cccc2ccccc12]

In some embodiments, R₁ is

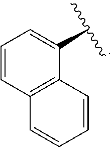

In some embodiments, R₁ is

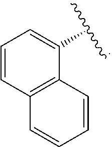

In some embodiments, R₁ is

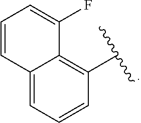 [SMILES: *c1cccc2cccc(F)c12]

In some embodiments, R₁ is

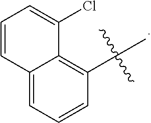 [SMILES: *c1cccc2cccc(Cl)c12]

In some embodiments, R₁ is

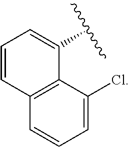

In some embodiments, R₁ is

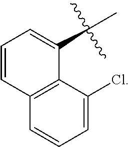

In some embodiments, R₁ is

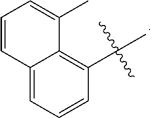 [SMILES: *c1cccc2cccc(C)c12]

In some embodiments, R₁ is

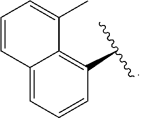

In some embodiments, R₁ is

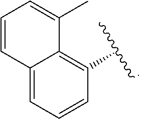

In some embodiments, R₁ is

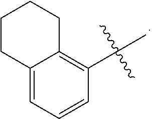 [SMILES: *c1cccc2c1CCCC2]

In some embodiments, R₁ is

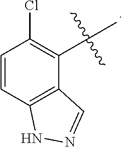 [SMILES: *c1c(Cl)ccc2[nH]ncc12]

In some embodiments, R₁ is

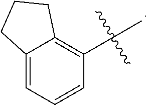 [SMILES: *c1cccc2c1CCC2]

In some embodiments, R₁ is

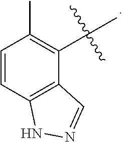 [SMILES: *c1c(C)ccc2[nH]ncc12]

In some embodiments, R₁ is

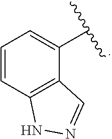 [SMILES: *c1cccc2[nH]ncc12]

In some embodiments, R₁ is

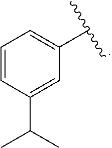 [SMILES: *c1cccc(C(C)C)c1]

In some embodiments, R₁ is

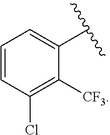 [SMILES: *c1cccc(Cl)c1C(F)(F)F]

In some embodiments, R₁ is

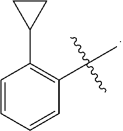 [SMILES: *c1ccccc1C1CC1]

In some embodiments, R₁ is

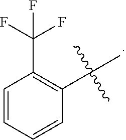 [SMILES: *c1ccccc1C(F)(F)F]

In some embodiments, R₁ is

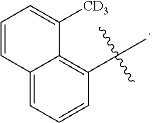 [SMILES: *c1cccc2cccc(C([2H])([2H])[2H])c12]

In some embodiments, R₁ is

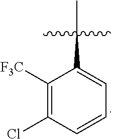

In some embodiments, R₁ is

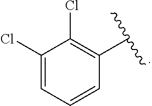 [SMILES: *c1cccc(Cl)c1Cl]

In some embodiments, R₁ is

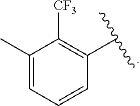 [SMILES: *c1cccc(C)c1C(F)(F)F]

In some embodiments, R₁ is

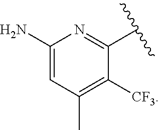 [SMILES: *c1nc(N)cc(C)c1C(F)(F)F]

In some embodiments, R₁ is

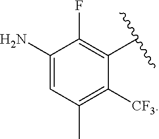 [SMILES: *c1c(F)c(N)cc(C)c1C(F)(F)F]

In some embodiments,

R₁ is

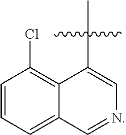 [SMILES: *c1cncc2cccc(Cl)c12]

In some embodiments, R₁ is

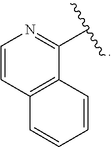 [SMILES: *c1nccc2ccccc12]

In some embodiments, R₁ is

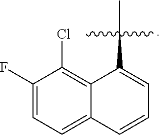

In some embodiments, R₁ is

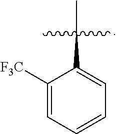

In some embodiments, R₁ is

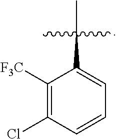

In some embodiments, R₁ is

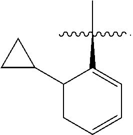

In some embodiments, R₅ is chosen from halogen, C₁-C₃ alkyl, C₁-C₃haloalkyl, deuterated C₁-C₃ alkyl, amino group, and —CN. In some embodiments, R₅ is a halogen. In some embodiments, R₅ is F. In some embodiments, R₅ is Cl. In some embodiments, R₅ is Br. In some embodiments, R₅ is I. In some embodiments, R₅ is a C₁-C₃ alkyl. In some embodiments, R₅ is —CH₃. In some embodiments, R₅ is —CH₂CH₃. In some embodiments, R₅ is C₂-alkynyl. In some embodiments, R₅ is

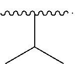 [SMILES: *C(C)C]

In some embodiments, R₅ is a C₁-C₃haloalkyl. In some embodiments, R₅ is —CF₃. In some embodiments, R₅ is deuterated C₁-C₃ alkyl. In some embodiments, R₅ is —CD₃. In some embodiments, R₅ is an amino group. In some embodiments, R₅ is —CN. In some embodiments, R₅ is

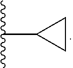 [SMILES: *C1CC1]

In some embodiments, R₂ is chosen from hydrogen, C₁-C₃alkyl, and cycloalkyl. In some embodiments, R₂ is chosen from hydrogen, C₁-C₄ alkyl, and C₃-C₄cycloalkyl. In some embodiments, R₂ is hydrogen. In some embodiments, R₂ is a C₁-C₃alkyl. In some embodiments, R₂ is —CH₃. In some embodiments, R₂ is

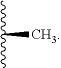

In some embodiments, R₂ is

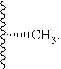

In some embodiments, R₂ is —CH₂CH₃. In some embodiments, R₂ is C₃-C₄ cycloalkyl.

R₃ is chosen from C₁-C₄ alkyl, C₁-C₄ alkoxyalkyl, C₁-C₄ hydroxyalkyl, C₁-C₆ dihydroxyalkyl, C₁-C₆ dialkylaminoalkyl, cycloalkyl, heterocycle, and, heterocycloalkyl, each of which is independently substituted with 0, 1, 2, or 3 R₆. In In some embodiments, R₃ is chosen from C₃-C₆cycloalkyl, C₃-C₆ heterocycle, and C₃-C₆heterocycloalkyl. In some embodiments, R₃ is chosen from H, C₁-C₆ alkyl, C₁-C₆ alkoxyalkyl, C₁-C₆ hydroxyalkyl, C₁-C₆ dihydroxyalkyl, C₁-C₆ dialkylaminoalkyl, C₃-C₆ cycloalkyl, C₃-C₆ heterocycle, C₃-C₆ heterocyclicalkyl, aryl, and heteroaryl.

In some embodiments, R₃ is chosen from

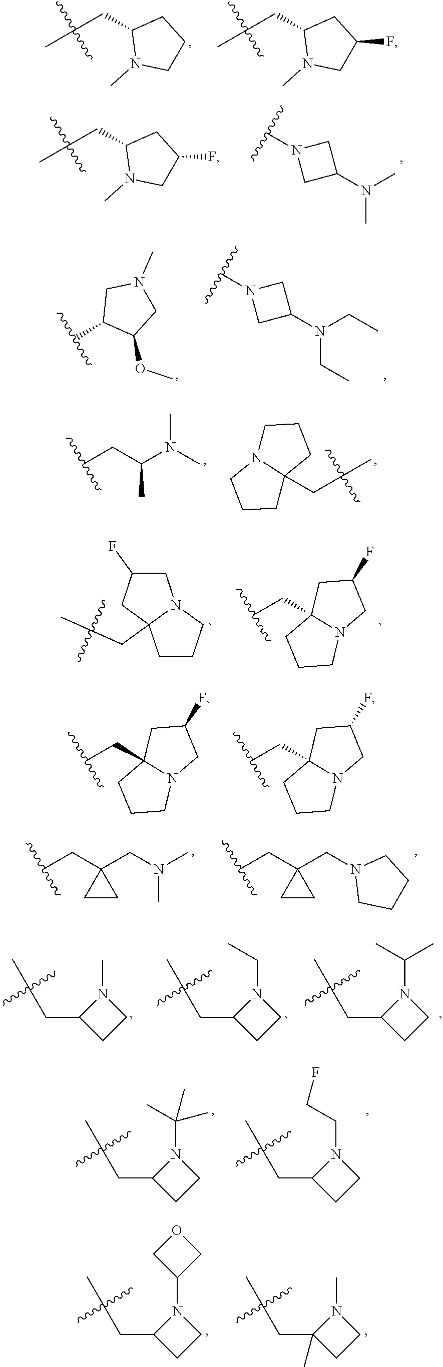

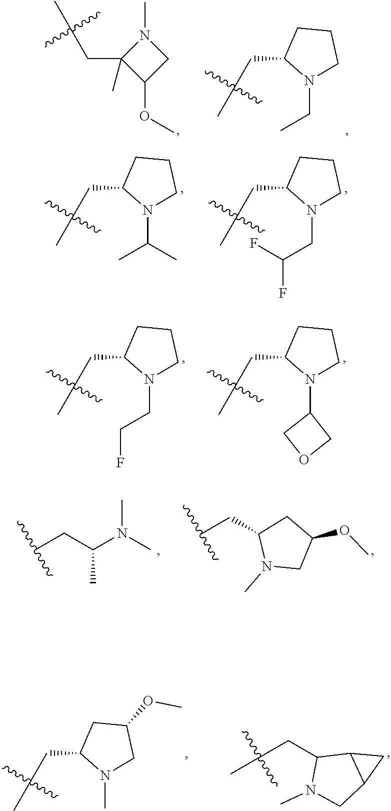

and

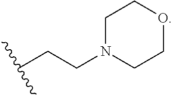 [SMILES: *CCN1CCOCC1]

In some embodiments, R₃ is

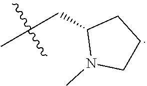 [SMILES: *C[C@@H]1CCCN1C]

In some embodiments,

R₃ is

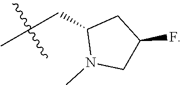 [SMILES: *C[C@@H]1C[C@@H](F)CN1C]

In some embodiments, R₃ is

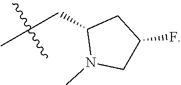 [SMILES: *C[C@@H]1C[C@H](F)CN1C]

In some embodiments, R₃ is

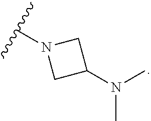 [SMILES: *N1CC(N(C)C)C1]

In some embodiments, R₃ is

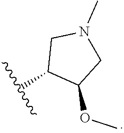 [SMILES: *[C@@H]1CN(C)C[C@H]1OC]

In some embodiments, R₃ is

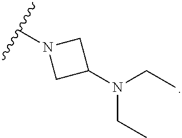 [SMILES: *N1CC(N(CC)CC)C1]

In some embodiments, R₃ is

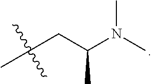 [SMILES: *C[C@H](C)N(C)C]

In some embodiments, R₃ is

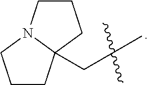 [SMILES: *CC12CCCN1CCC2]

In some embodiments, R₃ is

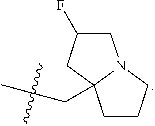 [SMILES: *CC12CCCN1CC(F)C2]

In some emboduments, R₃ is

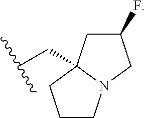 [SMILES: *C[C@@]12CCCN1C[C@H](F)C2]

In some embodiments, R₃ is

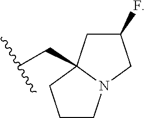 [SMILES: *C[C@]12CCCN1C[C@H](F)C2]

In some enbodiments, R₃ is

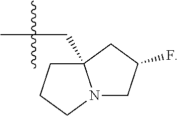 [SMILES: *C[C@@]12CCCN1C[C@@H](F)C2]

In some embodiments, R₃ is

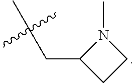 [SMILES: *CC1CCN1C]

In some embodiments, R₃ is

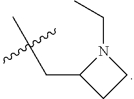 [SMILES: *CC1CCN1CC]

In some embodiments, R₃ is

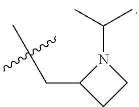 [SMILES: *CC1CCN1C(C)C]

In some embodiments, R₃ is

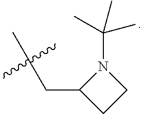 [SMILES: *CC1CCN1C(C)(C)C]

In some embodiments, R₃ is

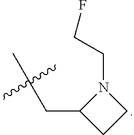 [SMILES: *CC1CCN1CCF]

In some embodiments, R₃ is

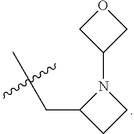 [SMILES: *CC1CCN1C1COC1]

In some embodiments, R₃ is

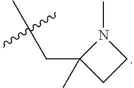 [SMILES: *CC1(C)CCN1C]

In some embodiments, R₃ is

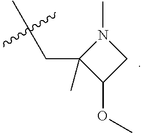 [SMILES: *CC1(C)C(OC)CN1C ; "."]

In some embodiments, R₃ is

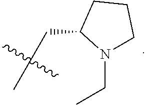 [SMILES: *C[C@@H]1CCCN1CC ; "."]

In some embodiments, R₃ is

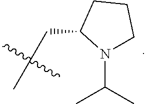 [SMILES: *C[C@@H]1CCCN1C(C)C ; "."]

In some embodiments, R₃ is

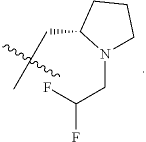 [SMILES: *C[C@@H]1CCCN1CC(F)F ; "."]

In some embodiments, R₃ is

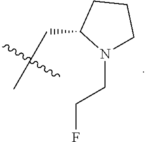 [SMILES: *C[C@@H]1CCCN1CCF ; "."]

In some embodiments, R₃ is

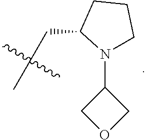 [SMILES: *C[C@@H]1CCCN1C1COC1 ; "."]

In some embodiments, R₃ is

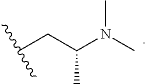 [SMILES: *C[C@@H](C)N(C)C ; "."]

In some embodiments, R₃ is

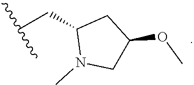 [SMILES: *C[C@@H]1C[C@@H](OC)CN1C ; "."]

In some embodiments, R₃ is

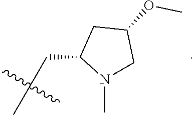 [SMILES: *C[C@@H]1C[C@H](OC)CN1C ; "."]

In some embodiments, R₃ is

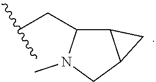 [SMILES: *CC1C2CC2CN1C ; "."]

In some embodiments, R₃ is

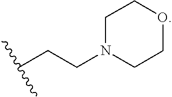 [SMILES: *CCN1CCOCC1]

In some embodiments, R₆ is chosen from halogen, C₁-C₃ alkyl, C₁-C₃haloalkyl, C₁-₄alkoxy, dialkylamino group, cycloalkyl, heterocycle, heterocycloalkyl, and heteroaryl. In some embodiments, R₆ is a halogen. In some embodiments, R₆ is F. In some embodiments, R₆ is Cl. In some embodiments, R₆ is a C₁-C₃ alkyl. In some embodiments, R₆ is —CH₃. In some embodiments, R₆ is —CH₂CH₃. In some embodiments, R₆ is

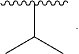 [SMILES: *C(C)C ; "."]

In some embodiments, R₆ is a C₁-C₃haloalkyl. In some embodiments, R₆ is

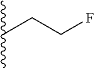 [SMILES: *CCF]

In some embodiments, R₆ is

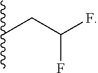 [SMILES: *CC(F)F]

In some embodiments, R₆ is a heterocycle. In some embodiments, R₆ is

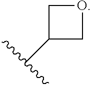 [SMILES: *C1COC1]

In some embodiments, R₆ is a C₁-₄alkoxy. In some embodiments, R₆ is a C₁-C₃alkoxy. In some embodiments, R₆ is

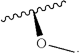

In some embodiments, R₆ is

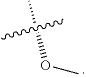

In some embodiments, R₆ is

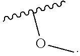 [SMILES: *OC]

In some embodiments, R₆ is a dialkylamino group. In some embodiments, R₆ is

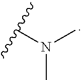 [SMILES: *N(C)C]

In some embodiments, R₆ is

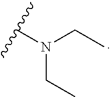 [SMILES: *N(CC)CC]

In some embodiments, R₄ is chosen from a C₂-C₆ alkenyl and C₂-C₆ alkynyl, wherein each of the C₂-C₆ alkenyl and C₂-C₆ alkynyl is independently substituted with H, halogen, CN, alkyl, dialkylaminoalkyl, and heterocyclic alkyl. In some embodiments, R₄ is chosen from —CH═CH₂,

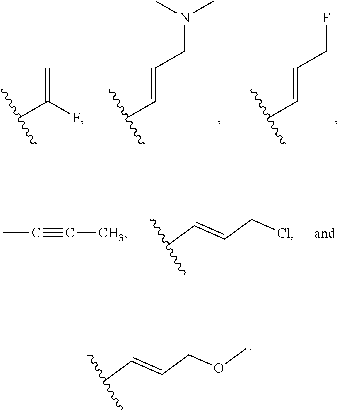

In some embodiments, R₄ is —CH═CH₂. In some embodiments, R₄ is

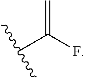 [SMILES: *C(=C)F]

In some embodiments, R₄ is

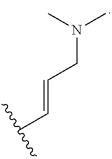 [SMILES: */C=C/CN(C)C]

In some embodiments, R₄ is

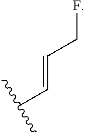 [SMILES: */C=C/CF]

In some embodiments, R₄ is —C≡C—CH₃. In some embodiments, R₄ is

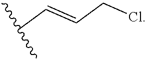 [SMILES: */C=C/CCl]

In some embodiments, R₄ is

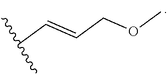 [SMILES: */C=C/COC]

In some embodiments, L is a bond (e.g. when L is a bond, it means L is absent and R₃ is attached directly) or O. In some embodiments, L is a bond. In some embodiments, L is O.

In some embodiments, Q is a 4-12 membered heteromonocyclic group, bridged or spiro heterocyclic group, wherein each of the heteromonocyclic group, bridged or spiro heterocyclic group is independently substituted with 0, 1, or 2 R₇.

In some embodiments, R₇ is a C₁-C₄ alkyl, wherein the C₁-C₄ alkyl is further substituted with hydrogen, halogen, —CN, ester, and carboxamide. In some embodiments, R₇ is a C₁-C₃alkyl. In some embodiments, R₇ is —CH₃. In some embodiments, the C₁-C₃alkyl is further substituted with halogen, —CN, and carboxamide. In some embodiments, the C₁-C₃alkyl is substituted with —CN. In some embodiments, R₇ is —CH₂—CN. In some embodiments, R₇ is

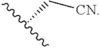

In some embodiments, R₇ is

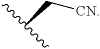

In some embodiments, the C₁-C₃alkyl is substituted with a carboxamide group.

In some embodiments, Q is chosen from

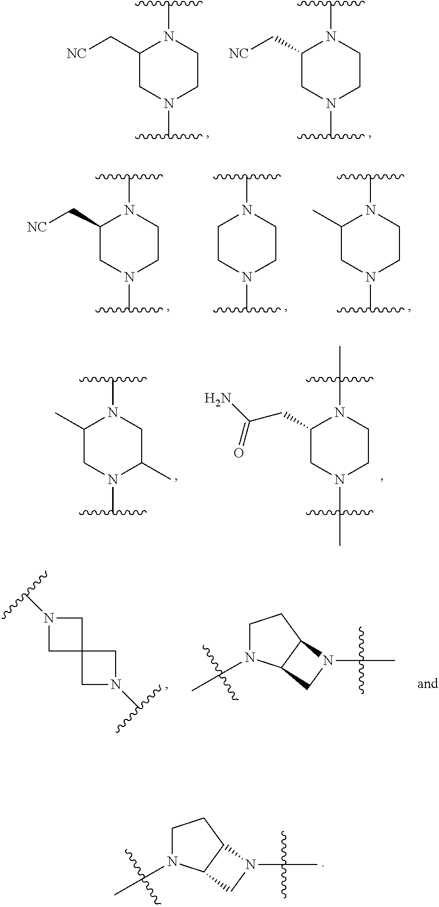

In some embodiments, Q is

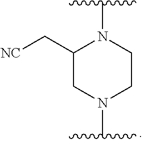 [SMILES: *N1CCN(*)C(CC#N)C1]

In some embodiments, Q is

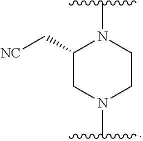 [SMILES: *N1CCN(*)[C@@H](CC#N)C1]

In some embodiments, Q is

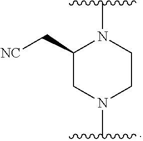 [SMILES: *N1CCN(*)[C@H](CC#N)C1]

In some embodiments, Q is

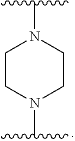 [SMILES: *N1CCN(*)CC1]

In some embodiments, Q is

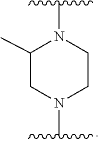

In some embodiments, Q is

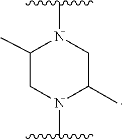

In some embodiments, Q is

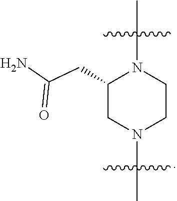

In some embodiments, Q is

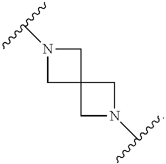 [SMILES: *N1CC2(C1)CN(*)C2]

In some embodiments, Q is

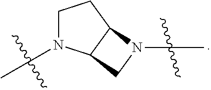 [SMILES: *N1CC[C@@H]2[C@H]1CN2*]

In some embodiments, Q is

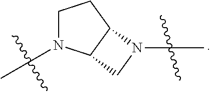 [SMILES: *N1CC[C@H]2[C@@H]1CN2*]

In some embodiments, provided herein is a compound chosen from the compounds listed in Table 1 or a tautomer, stereoisomer or a mixture of stereoisomers, or a pharmaceutically acceptable salt, or hydrate, or deuterated derivative of any of the foregoing.

TABLE 1
Exemplary Compounds of the Present Disclosure

| Example | | |
|---|---|---|
| # | Chemical Structure | IUPAC Name |
| 1 | 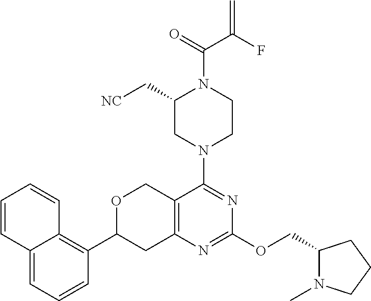 [SMILES: C=C(F)C(=O)N1CCN(c2nc(OC[C@@H]3CCCN3C)nc3c2COC(c2cccc4ccccc24)C3)C[C@@H]1CC#N] | 2-((2S)-1-(2-fluoroacryloyl)-4-(2-(((S)-1- methylpyrrolidin-2-yl)methoxy)-7-(naphthalen-1-yl)- 7,8-dihydro-5H-pyrano[4,3-d]pyrimidin-4- yl)piperazin-2-yl)acetonitrile |
| 2 | 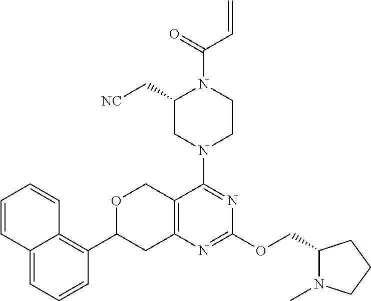 [SMILES: C=CC(=O)N1CCN(c2nc(OC[C@@H]3CCCN3C)nc3c2COC(c2cccc4ccccc24)C3)C[C@@H]1CC#N] | 2-((2S)-1-acryloyl-4-(2-(((S)-1-methylpyrrolidin-2- yl)methoxy)-7-(naphthalen-1-yl)-7,8-dihydro-5H- pyrano[4,3-d]pyrimidin-4-yl)piperazin-2- yl)acetonitrile |
| 3 | 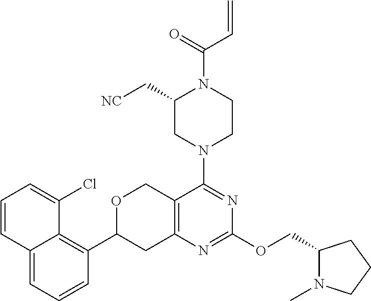 [SMILES: C=CC(=O)N1CCN(c2nc(OC[C@@H]3CCCN3C)nc3c2COC(c2cccc4cccc(Cl)c24)C3)C[C@@H]1CC#N] | 2-((2S)-1-acryloyl-4-(7-(8-chloronaphthalen-1-yl)-2- (((S)-1-methylpyrrolidin-2-yl)methoxy)-7,8-dihydro- 5H-pyrano[4,3-d]pyrimidin-4-yl)piperazin-2- yl)acetonitrile |
| 4 | 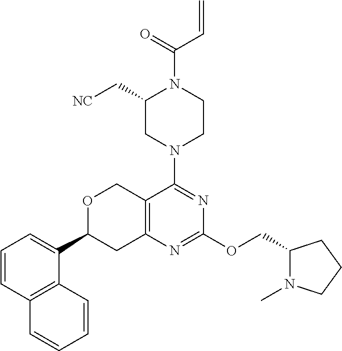 [SMILES: C=CC(=O)N1CCN(c2nc(OC[C@@H]3CCCN3C)nc3c2CO[C@H](c2cccc4ccccc24)C3)C[C@@H]1CC#N] | 2-((S)-1-acryloyl-4-((S)-2-(((S)-1-methylpyrrolidin-2- yl)methoxy)-7-(naphthalen-1-yl)-7,8-dihydro-5H- pyrano[4,3-d]pyrimidin-4-yl)piperazin-2- yl)acetonitrile |
| 5 | 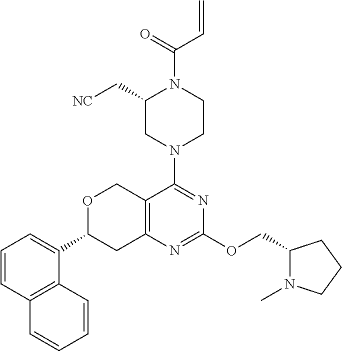 [SMILES: C=CC(=O)N1CCN(c2nc(OC[C@@H]3CCCN3C)nc3c2CO[C@@H](c2cccc4ccccc24)C3)C[C@@H]1CC#N] | 2-((S)-1-acryloyl-4-((R)-2-(((S)-1-methylpyrrolidin-2- yl)methoxy)-7-(naphthalen-1-yl)-7,8-dihydro-5H- pyrano[4,3-d]pyrimidin-4-yl)piperazin-2- yl)acetonitrile |
| 6 | 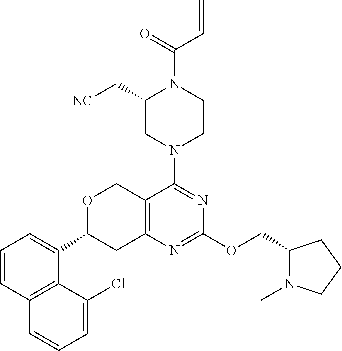 [SMILES: C=CC(=O)N1CCN(c2nc(OC[C@@H]3CCCN3C)nc3c2CO[C@@H](c2cccc4cccc(Cl)c24)C3)C[C@@H]1CC#N] | 2-((S)-1-acryloyl-4-((R)-7-(8-chloronaphthalen-1-yl)- 2-(((S)-1-methylpyrrolidin-2-yl)methoxy)-7,8-dihydro- 5H-pyrano[4,3-d]pyrimidin-4-yl)piperazin-2- yl)acetonitrile |
| 7 | 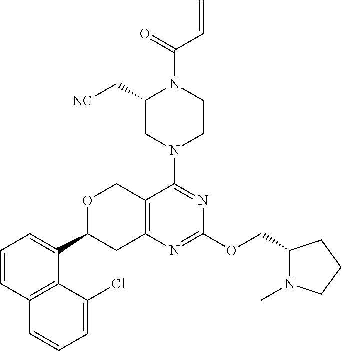 [SMILES: C=CC(=O)N1CCN(c2nc(OC[C@@H]3CCCN3C)nc3c2CO[C@H](c2cccc4cccc(Cl)c24)C3)C[C@@H]1CC#N] | 2-((S)-1-acryloyl-4-((S)-7-(8-chloronaphthalen-1-yl)- 2-(((S)-1-methylpyrrolidin-2-yl)methoxy)-7,8-dihydro- 5H-pyrano[4,3-d]pyrimidin-4-yl)piperazin-2- yl)acetonitrile |
| 8 | 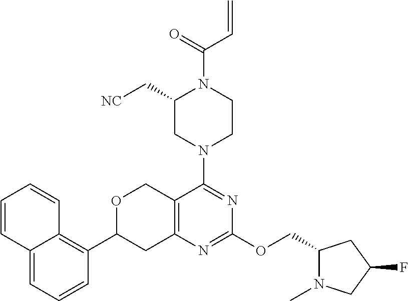 [SMILES: C=CC(=O)N1CCN(c2nc(OC[C@@H]3C[C@@H](F)CN3C)nc3c2COC(c2cccc4ccccc24)C3)C[C@@H]1CC#N] | 2-((2S)-1-acryloyl-4-(2-(((2S,4R)-4-fluoro-1- methylpyrrolidin-2-yl)methoxy)-7-(naphthalen-1-yl)- 7,8-dihydro-5H-pyrano[4,3-d]pyrimidin-4- yl)piperazin-2-yl)acetonitrile |
| 9 | 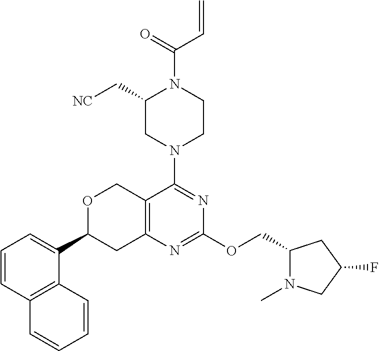 [SMILES: C=CC(=O)N1CCN(c2nc(OC[C@@H]3C[C@H](F)CN3C)nc3c2CO[C@H](c2cccc4ccccc24)C3)C[C@@H]1CC#N] | 2-((S)-1-acryloyl-4-((S)-2-(((2S,4S)-4-fluoro-1- methylpyrrolidin-2-yl)methoxy)-7-(naphthalen-1-yl)- 7,8-dihydro-5H-pyrano[4,3-d]pyrimidin-4- yl)piperazin-2-yl)acetonitrile |
| 10 | 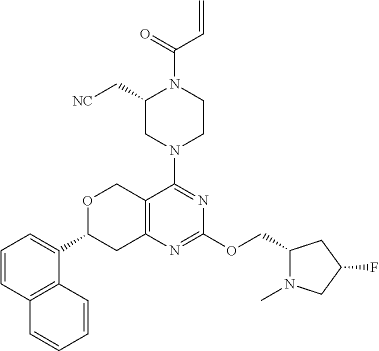 [SMILES: C=CC(=O)N1CCN(c2nc(OC[C@@H]3C[C@H](F)CN3C)nc3c2CO[C@@H](c2cccc4ccccc24)C3)C[C@@H]1CC#N] | 2-((S)-1-acryloyl-4-((R)-2-(((2S,4S)-4-fluoro-1- methylpyrrolidin-2-yl)methoxy)-7-(naphthalen-1-yl)- 7,8-dihydro-5H-pyrano[4,3-d]pyrimidin-4- yl)piperazin-2-yl)acetonitrile |
| 11 | 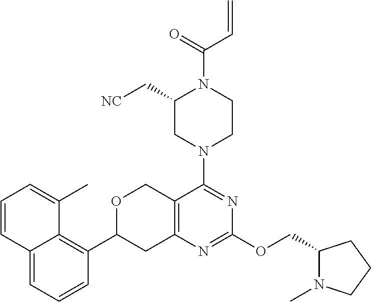 [SMILES: C=CC(=O)N1CCN(c2nc(OC[C@@H]3CCCN3C)nc3c2COC(c2cccc4cccc(C)c24)C3)C[C@@H]1CC#N] | 2-((2S)-1-acryloyl-4-(7-(8-methylnaphthalen-1-yl)-2- (((S)-1-methylpyrrolidin-2-yl)methoxy)-7,8-dihydro- 5H-pyrano[4,3-d]pyrimidin-4-yl)piperazin-2- yl)acetonitrile |
| 12 | 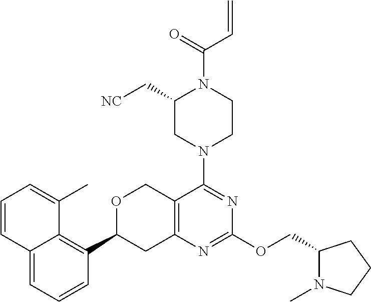 [SMILES: C=CC(=O)N1CCN(c2nc(OC[C@@H]3CCCN3C)nc3c2CO[C@H](c2cccc4cccc(C)c24)C3)C[C@@H]1CC#N] | 2-((S)-1-acryloyl-4-((S)-7-(8-methylnaphthalen-1-yl)- 2-(((S)-1-methylpyrrolidin-2-yl)methoxy)-7,8-dihydro- 5H-pyrano[4,3-d]pyrimidin-4-yl)piperazin-2- yl)acetonitrile |
| 13 | 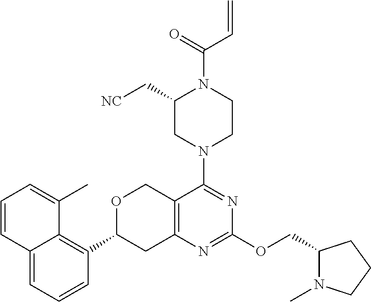 [SMILES: C=CC(=O)N1CCN(c2nc(OC[C@@H]3CCCN3C)nc3c2CO[C@@H](c2cccc4cccc(C)c24)C3)C[C@@H]1CC#N] | 2-((S)-1-acryloyl-4-((R)-7-(8-methylnaphthalen-1-yl)- 2-(((S)-1-methylpyrrolidin-2-yl)methoxy)-7,8-dihydro- 5H-pyrano[4,3-d]pyrimidin-4-yl)piperazin-2- yl)acetonitrile |
| 14 | 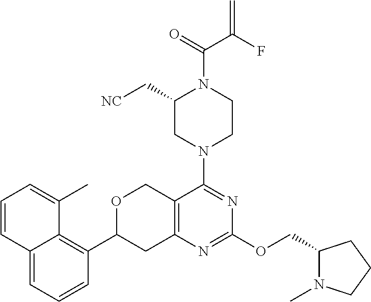 [SMILES: C=C(F)C(=O)N1CCN(c2nc(OC[C@@H]3CCCN3C)nc3c2COC(c2cccc4cccc(C)c24)C3)C[C@@H]1CC#N] | 2-((2S)-1-(2-fluoroacryloyl)-4-(7-(8- methylnaphthalen-1-yl)-2-(((S)-1-methylpyrrolidin-2- yl)methoxy)-7,8-dihydro-5H-pyrano[4,3-d]pyrimidin- 4-yl)piperazin-2-yl)acetonitrile |
| 15 | 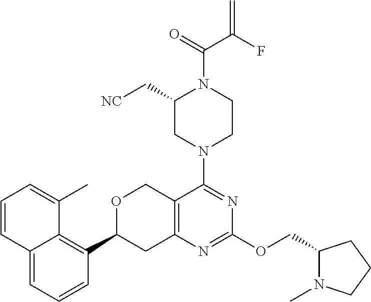 [SMILES: C=C(F)C(=O)N1CCN(c2nc(OC[C@@H]3CCCN3C)nc3c2CO[C@H](c2cccc4cccc(C)c24)C3)C[C@@H]1CC#N] | 2-((S)-1-(2-fluoroacryloyl)-4-((S)-7-(8- methylnaphthalen-1-yl)-2-(((S)-1-methylpyrrolidin-2- yl)methoxy)-7,8-dihydro-5H-pyrano[4,3-d]pyrimidin- 4-yl)piperazin-2-yl)acetonitrile |
| 16 | 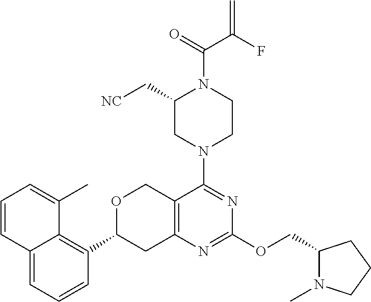 [SMILES: C=C(F)C(=O)N1CCN(c2nc(OC[C@@H]3CCCN3C)nc3c2CO[C@@H](c2cccc4cccc(C)c24)C3)C[C@@H]1CC#N] | 2-((S)-1-(2-fluoroacryloyl)-4-((R)-7-(8- methylnaphthalen-1-yl)-2-(((S)-1-methylpyrrolidin-2- yl)methoxy)-7,8-dihydro-5H-pyrano[4,3-d]pyrimidin- 4-yl)piperazin-2-yl)acetonitrile |
| 17 | 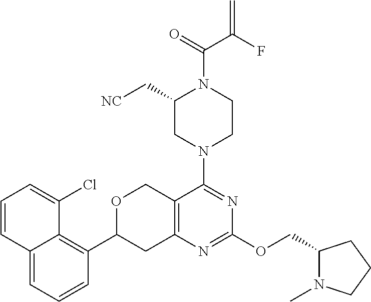 [SMILES: C=C(F)C(=O)N1CCN(c2nc(OC[C@@H]3CCCN3C)nc3c2COC(c2cccc4cccc(Cl)c24)C3)C[C@@H]1CC#N] | 2-((2S)-4-(7-(8-chloronaphthalen-1-yl)-2-(((S)-1- methylpyrrolidin-2-yl)methoxy)-7,8-dihydro-5H- pyrano[4,3-d]pyrimidin-4-yl)-1-(2- fluoroacryloyl)piperazin-2-yl)acetonitrile |
| 18 | 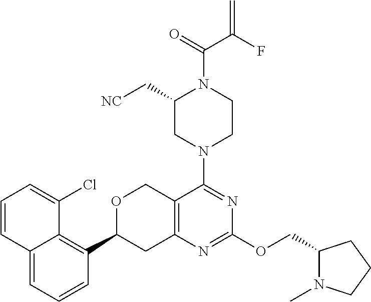 [SMILES: C=C(F)C(=O)N1CCN(c2nc(OC[C@@H]3CCCN3C)nc3c2CO[C@H](c2cccc4cccc(Cl)c24)C3)C[C@@H]1CC#N] | 2-((S)-4-((S)-7-(8-chloronaphthalen-1-yl)-2-(((S)-1- methylpyrrolidin-2-yl)methoxy)-7,8-dihydro-5H- pyrano[4,3-d]pyrimidin-4-yl)-1-(2- fluoroacryloyl)piperazin-2-yl)acetonitrile |
| 19 | 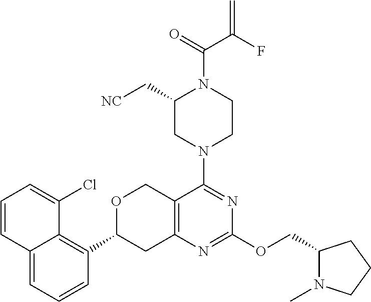 [SMILES: C=C(F)C(=O)N1CCN(c2nc(OC[C@@H]3CCCN3C)nc3c2CO[C@@H](c2cccc4cccc(Cl)c24)C3)C[C@@H]1CC#N] | 2-((S)-4-((R)-7-(8-chloronaphthalen-1-yl)-2-(((S)-1- methylpyrrolidin-2-yl)methoxy)-7,8-dihydro-5H- pyrano[4,3-d]pyrimidin-4-yl)-1-(2- fluoroacryloyl)piperazin-2-yl)acetonitrile |
| 20 | 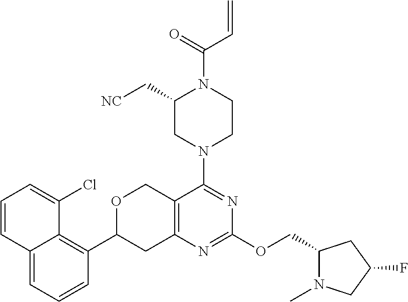 [SMILES: C=CC(=O)N1CCN(c2nc(OC[C@@H]3C[C@H](F)CN3C)nc3c2COC(c2cccc4cccc(Cl)c24)C3)C[C@@H]1CC#N] | 2-((2S)-1-acryloyl-4-(7-(8-chloronaphthalen-1-yl)-2- (((2S,4S)-4-fluoro-1-methylpyrrolidin-2-yl)methoxy)- 7,8-dihydro-5H-pyrano[4,3-d]pyrimidin-4- yl)piperazin-2-yl)acetonitrile |
| 21 | 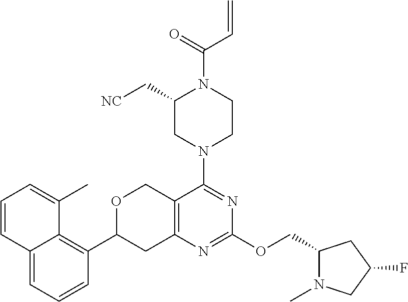 [SMILES: C=CC(=O)N1CCN(c2nc(OC[C@@H]3C[C@H](F)CN3C)nc3c2COC(c2cccc4cccc(C)c24)C3)C[C@@H]1CC#N] | 2-((2S)-1-acryloyl-4-(2-(((2S,4S)-4-fluoro-1- methylpyrrolidin-2-yl)methoxy)-7-(8- methylnaphthalen-1-yl)-7,8-dihydro-5H-pyrano[4,3- d]pyrimidin-4-yl)piperazin-2-yl)acetonitrile |
| 22 | 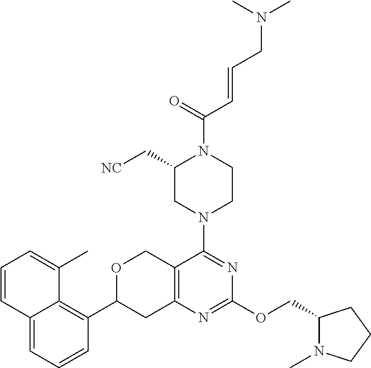 [SMILES: Cc1cccc2cccc(C3Cc4nc(OC[C@@H]5CCCN5C)nc(N5CCN(C(=O)/C=C/CN(C)C)[C@@H](CC#N)C5)c4CO3)c12] | 2-((2S)-1-((E)-4-(dimethylamino)but-2-enoyl)-4-(7- (8-methylnaphthalen-1-yl)-2-(((S)-1-methylpyrrolidin- 2-yl)methoxy)-7,8-dihydro-5H-pyrano[4,3- d]pyrimidin-4-yl)piperazin-2-yl)acetonitrile |
| 23 | 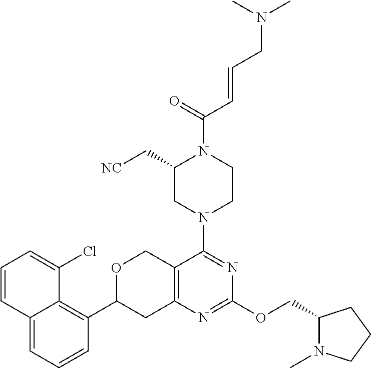 [SMILES: CN(C)C/C=C/C(=O)N1CCN(c2nc(OC[C@@H]3CCCN3C)nc3c2COC(c2cccc4cccc(Cl)c24)C3)C[C@@H]1CC#N] | 2-((2S)-4-(7-(8-chloronaphthalen-1-yl)-2-(((S)-1- methylpyrrolidin-2-yl)methoxy)-7,8-dihydro-5H- pyrano[4,3-d]pyrimidin-4-yl)-1-((E)-4- (dimethylamino)but-2-enoyl)piperazin- 2-yl)acetonitrile |
| 24 | 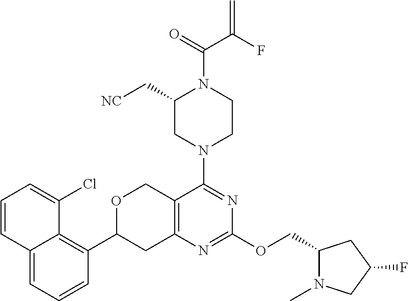 [SMILES: C=C(F)C(=O)N1CCN(c2nc(OC[C@@H]3C[C@H](F)CN3C)nc3c2COC(c2cccc4cccc(Cl)c24)C3)C[C@@H]1CC#N] | 2-((2S)-4-(7-(8-chloronaphthalen-1-yl)-2-(((2S,4S)-4- fluoro-1-methylpyrrolidin-2-yl)methoxy)-7,8-dihydro- 5H-pyrano[4,3-d]pyrimidin-4-yl)-1-(2- fluoroacryloyl)piperazin-2-yl)acetonitrile |
| 25 | 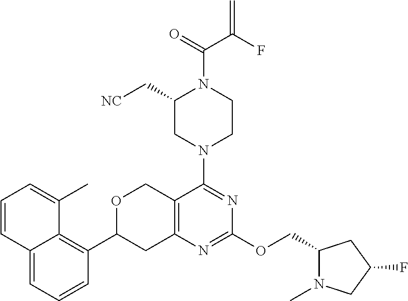 [SMILES: C=C(F)C(=O)N1CCN(c2nc(OC[C@@H]3C[C@H](F)CN3C)nc3c2COC(c2cccc4cccc(C)c24)C3)C[C@@H]1CC#N] | 2-((2S)-4-(2-(((2S,4S)-4-fluoro-1-methylpyrrolidin-2- yl)methoxy)-7-(8-methylnaphthalen-1-yl)-7,8- dihydro-5H-pyrano[4,3-d]pyrimidin-4-yl)-1-(2- fluoroacryloyl)piperazin-2-yl)acetonitrile |
| 26 | 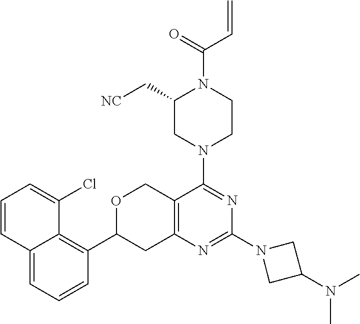 [SMILES: C=CC(=O)N1CCN(c2nc(N3CC(N(C)C)C3)nc3c2COC(c2cccc4cccc(Cl)c24)C3)C[C@@H]1CC#N] | 2-((2S)-1-acryloyl-4-(7-(8-chloronaphthalen-1-yl)-2- (3-(dimethylamino)azetidin-1-yl)-7,8-dihydro-5H- pyrano[4,3-d]pyrimidin-4-yl)piperazin-2- yl)acetonitrile |
| 27 | 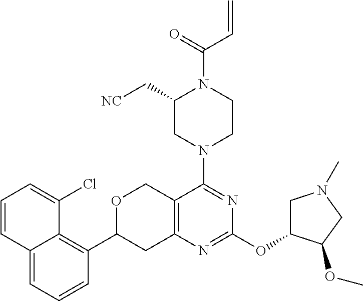 [SMILES: C=CC(=O)N1CCN(c2nc(O[C@@H]3CN(C)C[C@H]3OC)nc3c2COC(c2cccc4cccc(Cl)c24)C3)C[C@@H]1CC#N] | 2-((2S)-1-acryloyl-4-(7-(8-chloronaphthalen-1-yl)-2- (((3R,4R)-4-methoxy-1-methylpyrrolidin-3-yl)oxy)- 7,8-dihydro-5H-pyrano[4,3-d]pyrimidin-4- yl)piperazin-2-yl)acetonitrile |
| 28 | 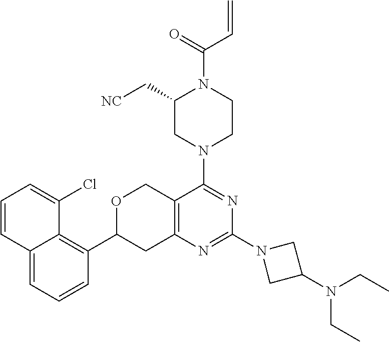 [SMILES: C=CC(=O)N1CCN(c2nc(N3CC(N(CC)CC)C3)nc3c2COC(c2cccc4cccc(Cl)c24)C3)C[C@@H]1CC#N] | 2-((2S)-1-acryloyl-4-(7-(8-chloronaphthalen-1-yl)-2- (3-(diethylamino)azetidin-1-yl)-7,8-dihydro-5H- pyrano[4,3-d]pyrimidin-4-yl)piperazin-2- yl)acetonitrile |
| 29 | 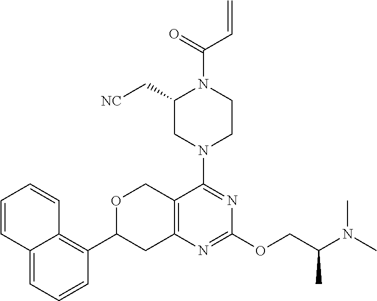 [SMILES: C=CC(=O)N1CCN(c2nc(OC[C@H](C)N(C)C)nc3c2COC(c2cccc4ccccc24)C3)C[C@@H]1CC#N] | 2-((2S)-1-acryloyl-4-(2-((S)-2- (dimethylamino)propoxy)-7-(naphthalen-1-yl)-7,8- dihydro-5H-pyrano[4,3-d]pyrimidin-4-yl)piperazin-2- yl)acetonitrile |
| 30 | 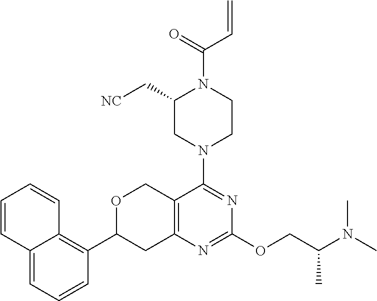 [SMILES: C=CC(=O)N1CCN(c2nc(OC[C@@H](C)N(C)C)nc3c2COC(c2cccc4ccccc24)C3)C[C@@H]1CC#N] | 2-((2S)-1-acryloyl-4-(2-((R)-2- (dimethylamino)propoxy)-7-(naphthalen-1-yl)-7,8- dihydro-5H-pyrano[4,3-d]pyrimidin-4-yl)piperazin-2- yl)acetonitrile |
| 31 | 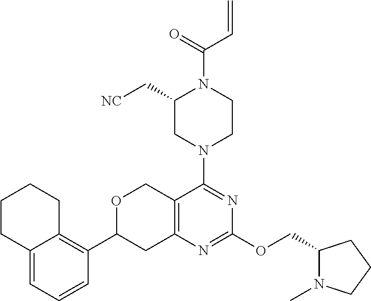 [SMILES: C=CC(=O)N1CCN(c2nc(OC[C@@H]3CCCN3C)nc3c2COC(c2cccc4c2CCCC4)C3)C[C@@H]1CC#N] | 2-((2S)-1-acryloyl-4-(2-(((S)-1-methylpyrrolidin-2- yl)methoxy)-7-(5,6,7,8-tetrahydronaphthalen-1-yl)- 7,8-dihydro-5H-pyrano[4,3-d]pyrimidin-4- yl)piperazin-2-yl)acetonitrile |
| 32 | 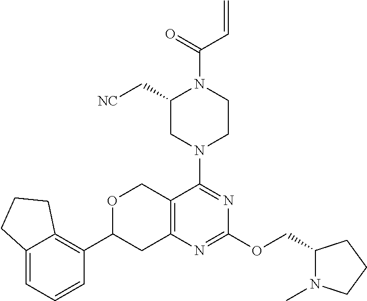 [SMILES: C=CC(=O)N1CCN(c2nc(OC[C@@H]3CCCN3C)nc3c2COC(c2cccc4c2CCC4)C3)C[C@@H]1CC#N] | 2-((2S)-1-acryloyl-4-(7-(2,3-dihydro-1H-inden-4-yl)- 2-(((S)-1-methylpyrrolidin-2-yl)methoxy)-7,8-dihydro- 5H-pyrano[4,3-d]pyrimidin-4-yl)piperazin-2- yl)acetonitrile |
| 33 | 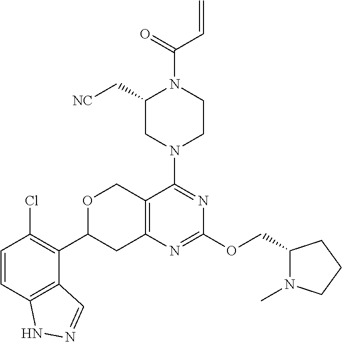 [SMILES: C=CC(=O)N1CCN(c2nc(OC[C@@H]3CCCN3C)nc3c2COC(c2c(Cl)ccc4[nH]ncc24)C3)C[C@@H]1CC#N] | 2-((2S)-1-acryloyl-4-(7-(5-chloro-1H-indazol-4-yl)-2- (((S)-1-methylpyrrolidin-2-yl)methoxy)-7,8-dihydro- 5H-pyrano[4,3-d]pyrimidin-4-yl)piperazin-2- yl)acetonitrile |
| 34 | 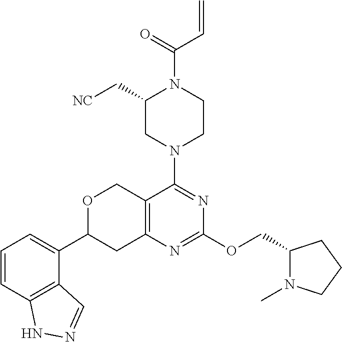 [SMILES: C=CC(=O)N1CCN(c2nc(OC[C@@H]3CCCN3C)nc3c2COC(c2cccc4[nH]ncc24)C3)C[C@@H]1CC#N] | 2-((2S)-4-(7-(1H-indazol-4-yl)-2-(((S)-1- methylpyrrolidin-2-yl)methoxy)-7,8-dihydro-5H- pyrano[4,3-d]pyrimidin-4-yl)-1-acryloylpiperazin-2- yl)acetonitrile |
| 35 | 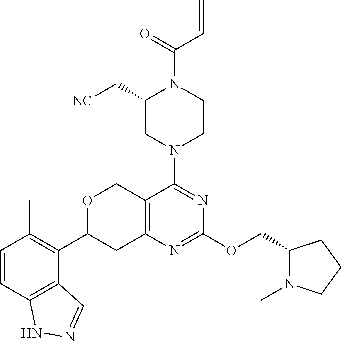 [SMILES: C=CC(=O)N1CCN(c2nc(OC[C@@H]3CCCN3C)nc3c2COC(c2c(C)ccc4[nH]ncc24)C3)C[C@@H]1CC#N] | 2-((2S)-1-acryloyl-4-(7-(5-methyl-1H-indazol-4-yl)-2- (((S)-1-methylpyrrolidin-2-yl)methoxy)-7,8-dihydro- 5H-pyrano[4,3-d]pyrimidin-4-yl)piperazin-2- yl)acetonitrile |
| 36 | 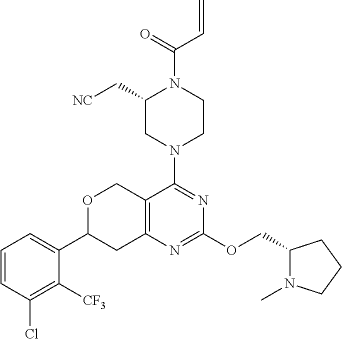 [SMILES: C=CC(=O)N1CCN(c2nc(OC[C@@H]3CCCN3C)nc3c2COC(c2cccc(Cl)c2C(F)(F)F)C3)C[C@@H]1CC#N] | 2-((2S)-1-acryloyl-4-(7-(3-chloro-2- (trifluoromethyl)phenyl)-2-(((S)-1-methylpyrrolidin-2- yl)methoxy)-7,8-dihydro-5H-pyrano[4,3-d]pyrimidin- 4-yl)piperazin-2-yl)acetonitrile |
| 37 | 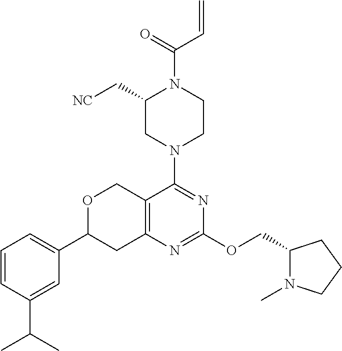 [SMILES: C=CC(=O)N1CCN(c2nc(OC[C@@H]3CCCN3C)nc3c2COC(c2cccc(C(C)C)c2)C3)C[C@@H]1CC#N] | 2-((2S)-1-acryloyl-4-(7-(3-isopropylphenyl)-2-(((S)-1- methylpyrrolidin-2-yl)methoxy)-7,8-dihydro-5H- pyrano[4,3-d]pyrimidin-4-yl)piperazin-2- yl)acetonitrile |
| 38 | 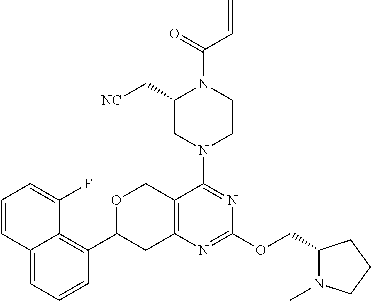 [SMILES: C=CC(=O)N1CCN(c2nc(OC[C@@H]3CCCN3C)nc3c2COC(c2cccc4cccc(F)c24)C3)C[C@@H]1CC#N] | 2-((2S)-1-acryloyl-4-(7-(8-fluoronaphthalen-1-yl)-2- (((S)-1-methylpyrrolidin-2-yl)methoxy)-7,8-dihydro- 5H-pyrano[4,3-d]pyrimidin-4-yl)piperazin-2- yl)acetonitrile |
| 39 | 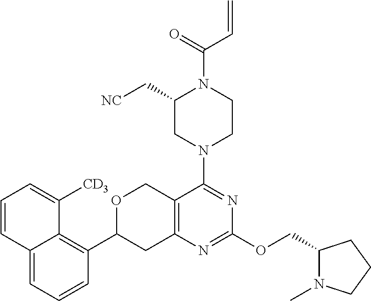 [SMILES: [2H]C([2H])([2H])c1cccc2cccc(C3Cc4nc(OC[C@@H]5CCCN5C)nc(N5CCN(C(=O)C=C)[C@@H](CC#N)C5)c4CO3)c12] | 2-((2S)-1-acryloyl-4-(7-(8-(methyl-d3)naphthalen- 1-yl)-2-(((S)-1-methylpyrrolidin-2-yl)methoxy)-7,8- dihydro-5H-pyrano[4,3-d]pyrimidin-4-yl)piperazin-2- yl)acetonitrile |
| 40 | 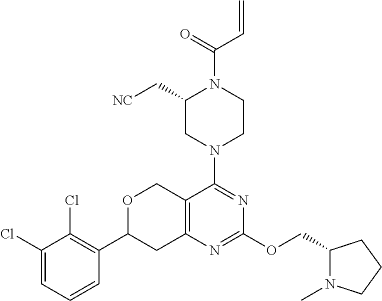 [SMILES: C=CC(=O)N1CCN(c2nc(OC[C@@H]3CCCN3C)nc3c2COC(c2cccc(Cl)c2Cl)C3)C[C@@H]1CC#N] | 2-((2S)-1-acryloyl-4-(7-(2,3-dichlorophenyl)-2-(((S)- 1-methylpyrrolidin-2-yl)methoxy)-7,8-dihydro-5H- pyrano[4,3-d]pyrimidin-4-yl)piperazin-2- yl)acetonitrile |
| 41 | 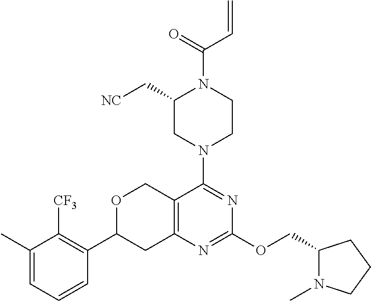 [SMILES: C=CC(=O)N1CCN(c2nc(OC[C@@H]3CCCN3C)nc3c2COC(c2cccc(C)c2C(F)(F)F)C3)C[C@@H]1CC#N] | 2-((2S)-1-acryloyl-4-(7-(3-methyl-2- (trifluoromethyl)phenyl)-2-(((S)-1-methylpyrrolidin-2- yl)methoxy)-7,8-dihydro-5H-pyrano[4,3-d]pyrimidin- 4-yl)piperazin-2-yl)acetonitrile |
| 42 | 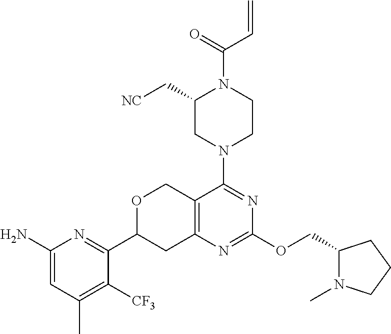 [SMILES: C=CC(=O)N1CCN(c2nc(OC[C@@H]3CCCN3C)nc3c2COC(c2nc(N)cc(C)c2C(F)(F)F)C3)C[C@@H]1CC#N] | 2-((2S)-1-acryloyl-4-(7-(6-amino-4-methyl-3- (trifluoromethyl)pyridin-2-yl)-2-(((S)-1- methylpyrrolidin-2-yl)methoxy)-7,8-dihydro-5H- pyrano[4,3-d]pyrimidin-4-yl)piperazin-2- yl)acetonitrile |
| 43 | 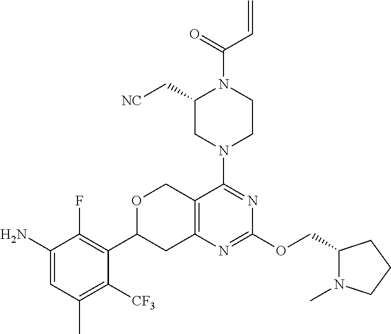 [SMILES: C=CC(=O)N1CCN(c2nc(OC[C@@H]3CCCN3C)nc3c2COC(c2c(F)c(N)cc(C)c2C(F)(F)F)C3)C[C@@H]1CC#N] | 2-((2S)-1-acryloyl-4-(7-(3-amino-2-fluoro-5-methyl-6- (trifluoromethyl)phenyl)-2-(((S)-1-methylpyrrolidin-2- yl)methoxy)-7,8-dihydro-5H-pyrano[4,3-d]pyrimidin- 4-yl)piperazin-2-yl)acetonitrile |
| 44 | 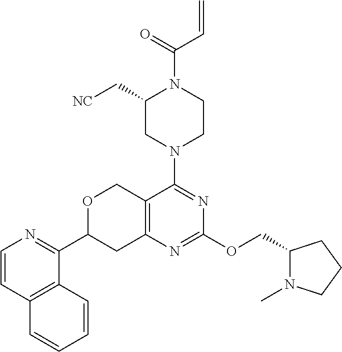 [SMILES: C=CC(=O)N1CCN(c2nc(OC[C@@H]3CCCN3C)nc3c2COC(c2nccc4ccccc24)C3)C[C@@H]1CC#N] | 2-((2S)-1-acryloyl-4-(7-(isoquinolin-1-yl)-2-(((S)-1- methylpyrrolidin-2-yl)methoxy)-7,8-dihydro-5H- pyrano[4,3-d]pyrimidin-4-yl)piperazin-2- yl)acetonitrile |
| 45 | 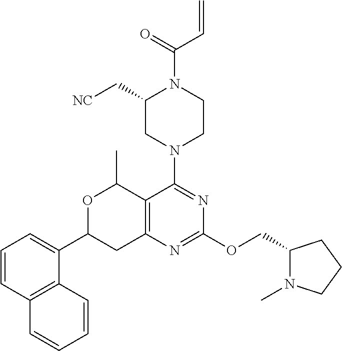 [SMILES: C=CC(=O)N1CCN(c2nc(OC[C@@H]3CCCN3C)nc3c2C(C)OC(c2cccc4ccccc24)C3)C[C@@H]1CC#N] | 2-((2S)-1-acryloyl-4-(5-methyl-2-(((S)-1- methylpyrrolidin-2-yl)methoxy)-7-(naphthalen-1-yl)- 7,8-dihydro-5H-pyrano[4,3-d]pyrimidin-4- yl)piperazin-2-yl)acetonitrile |
| 46 | 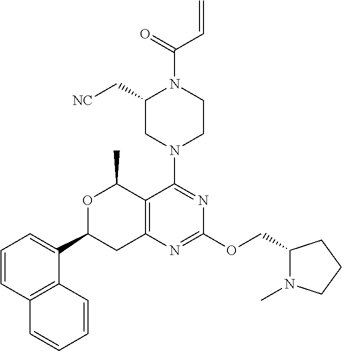 [SMILES: C=CC(=O)N1CCN(c2nc(OC[C@@H]3CCCN3C)nc3c2[C@H](C)O[C@H](c2cccc4ccccc24)C3)C[C@@H]1CC#N] | 2-((S)-1-acryloyl-4-((5S,7S)-5-methyl-2-(((S)-1- methylpyrrolidin-2-yl)methoxy)-7-(naphthalen-1-yl)- 7,8-dihydro-5H-pyrano[4,3-d]pyrimidin-4- yl)piperazin-2-yl)acetonitrile |
| 47 | 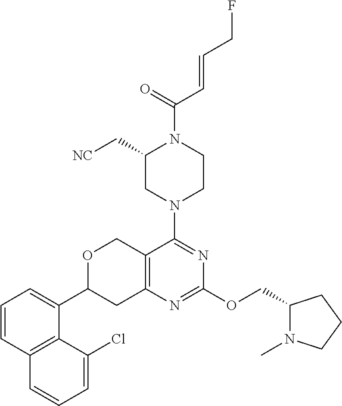 [SMILES: CN1CCC[C@H]1COc1nc2c(c(N3CCN(C(=O)/C=C/CF)[C@@H](CC#N)C3)n1)COC(c1cccc3cccc(Cl)c13)C2] | 2-((2S)-4-(7-(8-chloronaphthalen-1-yl)-2-(((S)-1- methylpyrrolidin-2-yl)methoxy)-7,8-dihydro-5H- pyrano[4,3-d]pyrimidin-4-yl)-1-((E)-4-fluorobut-2- enoyl)piperazin-2-yl)acetonitrile |
| 48 | 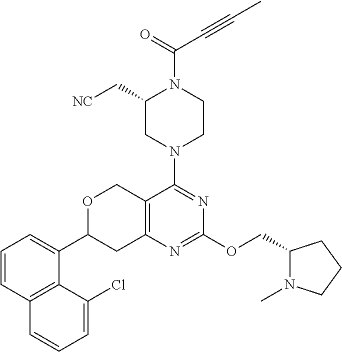 [SMILES: CC#CC(=O)N1CCN(c2nc(OC[C@@H]3CCCN3C)nc3c2COC(c2cccc4cccc(Cl)c24)C3)C[C@@H]1CC#N] | 2-((2S)-1-(but-2-ynoyl)-4-(7-(8-chloronaphthalen-1- yl)-2-(((S)-1-methylpyrrolidin-2-yl)methoxy)-7,8- dihydro-5H-pyrano[4,3-d]pyrimidin-4-yl)piperazin-2- yl)acetonitrile |
| 49 | 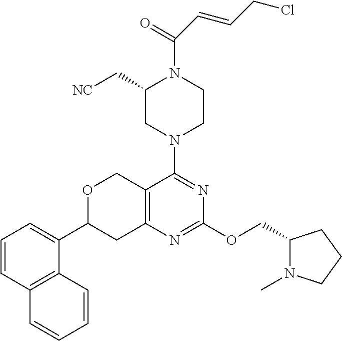 [SMILES: CN1CCC[C@H]1COc1nc2c(c(N3CCN(C(=O)/C=C/CCl)[C@@H](CC#N)C3)n1)COC(c1cccc3ccccc13)C2] | 2-((2S)-1-((E)-4-chlorobut-2-enoyl)-4-(2-(((S)-1- methylpyrrolidin-2-yl)methoxy)-7-(naphthalen-1-yl)- 7,8-dihydro-5H-pyrano[4,3-d]pyrimidin-4- yl)piperazin-2-yl)acetonitrile |
| 50 | 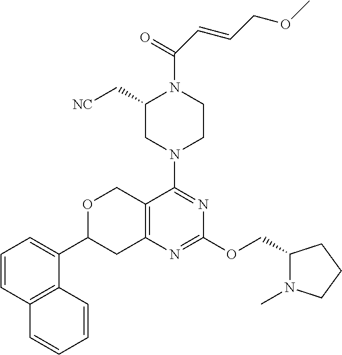 [SMILES: COC/C=C/C(=O)N1CCN(c2nc(OC[C@@H]3CCCN3C)nc3c2COC(c2cccc4ccccc24)C3)C[C@@H]1CC#N] | 2-((2S)-1-((E)-4-methoxybut-2-enoyl)-4-(2-(((S)-1- methylpyrrolidin-2-yl)methoxy)-7-(naphthalen-1-yl)- 7,8-dihydro-5H-pyrano[4,3-d]pyrimidin-4- yl)piperazin-2-yl)acetonitrile |
| 51 | 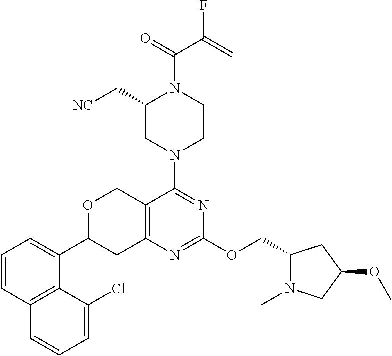 [SMILES: C=C(F)C(=O)N1CCN(c2nc(OC[C@@H]3C[C@@H](OC)CN3C)nc3c2COC(c2cccc4cccc(Cl)c24)C3)C[C@@H]1CC#N] | 2-((2S)-4-(7-(8-chloronaphthalen-1-yl)-2-(((2S,4R)-4- methoxy-1-methylpyrrolidin-2-yl)methoxy)-7,8- dihydro-5H-pyrano[4,3-d]pyrimidin-4-yl)-1-(2- fluoroacryloyl)piperazin-2-yl)acetonitrile |
| 52 | 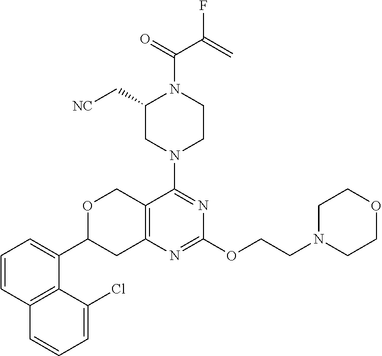 [SMILES: C=C(F)C(=O)N1CCN(c2nc(OCCN3CCOCC3)nc3c2COC(c2cccc4cccc(Cl)c24)C3)C[C@@H]1CC#N] | 2-((2S)-4-(7-(8-chloronaphthalen-1-yl)-2-(2- morpholinoethoxy)-7,8-dihydro-5H-pyrano[4,3- d]pyrimidin-4-yl)-1-(2-fluoroacryloyl)piperazin-2- yl)acetonitrile |
| 53 | 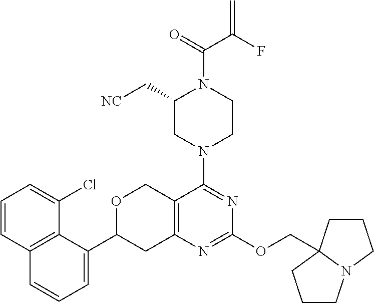 [SMILES: C=C(F)C(=O)N1CCN(c2nc(OCC34CCCN3CCC4)nc3c2COC(c2cccc4cccc(Cl)c24)C3)C[C@@H]1CC#N] | 2-((2S)-4-(7-(8-chloronaphthalen-1-yl)-2- ((tetrahydro-1H-pyrrolizin-7a(5H)-yl)methoxy)-7,8- dihydro-5H-pyrano[4,3-d]pyrimidin-4-yl)-1-(2- fluoroacryloyl)piperazin-2-yl)acetonitrile |
| 54 | 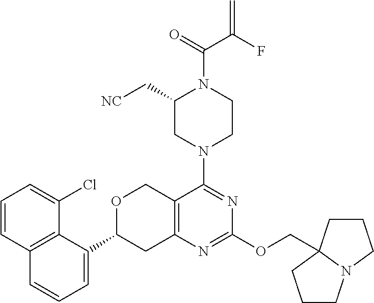 [SMILES: C=C(F)C(=O)N1CCN(c2nc(OCC34CCCN3CCC4)nc3c2CO[C@@H](c2cccc4cccc(Cl)c24)C3)C[C@@H]1CC#N] | 2-((S)-4-((R)-7-(8-chloronaphthalen-1-yl)-2- ((tetrahydro-1H-pyrrolizin-7a(5H)-yl)methoxy)-7,8- dihydro-5H-pyrano[4,3-d]pyrimidin-4-yl)-1-(2- fluoroacryloyl)piperazin-2-yl)acet |
| 55 | 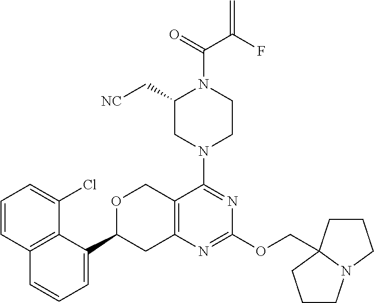 [SMILES: C=C(F)C(=O)N1CCN(c2nc(OCC34CCCN3CCC4)nc3c2CO[C@H](c2cccc4cccc(Cl)c24)C3)C[C@@H]1CC#N] | 2-((S)-4-((S)-7-(8-chloronaphthalen-1-yl)-2- ((tetrahydro-1H-pyrrolizin-7a(5H)-yl)methoxy)-7,8- dihydro-5H-pyrano[4,3-d]pyrimidin-4-yl)-1-(2- fluoroacryloyl)piperazin-2-yl)acetonitrile |
| 56 | 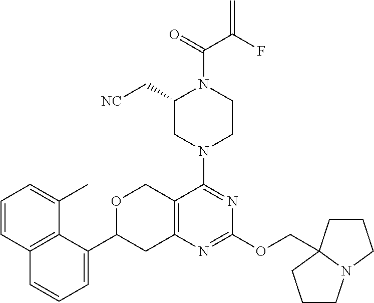 [SMILES: C=C(F)C(=O)N1CCN(c2nc(OCC34CCCN3CCC4)nc3c2COC(c2cccc4cccc(C)c24)C3)C[C@@H]1CC#N] | 2-((2S)-1-(2-fluoroacryloyl)-4-(7-(8- methylnaphthalen-1-yl)-2-((tetrahydro-1H-pyrrolizin- 7a(5H)-yl)methoxy)-7,8-dihydro-5H-pyrano[4,3- d]pyrimidin-4-yl)piperazin-2-yl)acetonitrile |
| 57 | 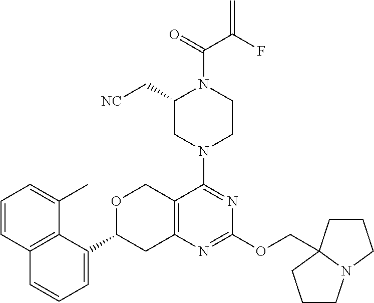 [SMILES: C=C(F)C(=O)N1CCN(c2nc(OCC34CCCN3CCC4)nc3c2CO[C@@H](c2cccc4cccc(C)c24)C3)C[C@@H]1CC#N] | 2-((S)-1-(2-fluoroacryloyl)-4-((R)-7-(8- methylnaphthalen-1-yl)-2-((tetrahydro-1H-pyrrolizin- 7a(5H)-yl)methoxy)-7,8-dihydro-5H-pyrano[4,3- d]pyrimidin-4-yl)piperazin-2-yl)acetonitrile |
| 58 | 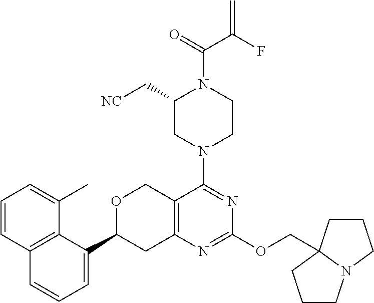 [SMILES: C=C(F)C(=O)N1CCN(c2nc(OCC34CCCN3CCC4)nc3c2CO[C@H](c2cccc4cccc(C)c24)C3)C[C@@H]1CC#N] | 2-((S)-1-(2-fluoroacryloyl)-4-((S)-7-(8- methylnaphthalen-1-yl)-2-((tetrahydro-1H-pyrrolizin- 7a(5H)-yl)methoxy)-7,8-dihydro-5H-pyrano[4,3- d]pyrimidin-4-yl)piperazin-2-yl)acetonitrile |
| 59 | 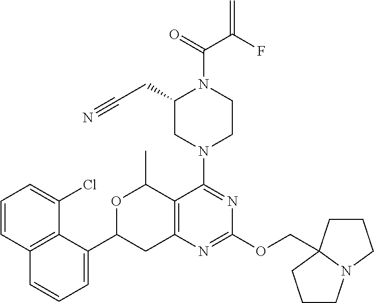 [SMILES: C=C(F)C(=O)N1CCN(c2nc(OCC34CCCN3CCC4)nc3c2C(C)OC(c2cccc4cccc(Cl)c24)C3)C[C@@H]1CC#N] | 2-((2S)-4-(7-(8-chloronaphthalen-1-yl)-5-methyl-2- ((tetrahydro-1H-pyrrolizin-7a(5H)-yl)methoxy)-7,8- dihydro-5H-pyrano[4,3-d]pyrimidin-4-yl)-1-(2- fluoroacryloyl)piperazin-2-yl)acetonitrile |
| 60 | 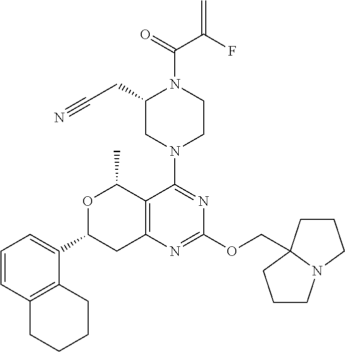 [SMILES: C=C(F)C(=O)N1CCN(c2nc(OCC34CCCN3CCC4)nc3c2[C@@H](C)O[C@@H](c2cccc4c2CCCC4)C3)C[C@@H]1CC#N] | 2-((S)-1-(2-fluoroacryloyl)-4-((5R,7R)-5-methyl-2- ((tetrahydro-1H-pyrrolizin-7a(5H)-yl)methoxy)-7- (5,6,7,8-tetrahydronaphthalen-1-yl)-7,8-dihydro-5H- pyrano[4,3-d]pyrimidin-4-yl)piperazin-2- yl)acetonitrile |
| 61 | 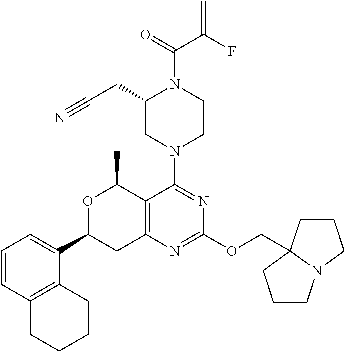 [SMILES: C=C(F)C(=O)N1CCN(c2nc(OCC34CCCN3CCC4)nc3c2[C@H](C)O[C@H](c2cccc4c2CCCC4)C3)C[C@@H]1CC#N] | 2-((S)-1-(2-fluoroacryloyl)-4-((5S,7S)-5-methyl-2- ((tetrahydro-1H-pyrrolizin-7a(5H)-yl)methoxy)-7- (5,6,7,8-tetrahydronaphthalen-1-yl)-7,8-dihydro-5H- pyrano[4,3-d]pyrimidin-4-yl)piperazin-2- yl)acetonitrile |
| 62 | 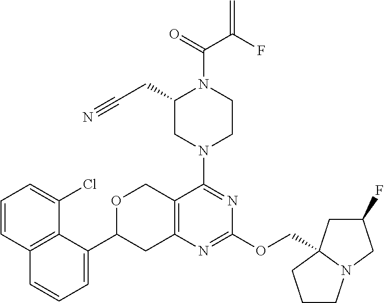 [SMILES: C=C(F)C(=O)N1CCN(c2nc(OC[C@@]34CCCN3C[C@H](F)C4)nc3c2COC(c2cccc4cccc(Cl)c24)C3)C[C@@H]1CC#N] | 2-((2S)-4-(7-(8-chloronaphthalen-1-yl)-2-(((2R,7aS)- 2-fluorotetrahydro-1H-pyrrolizin-7a(5H)-yl)methoxy)- 7,8-dihydro-5H-pyrano[4,3-d]pyrimidin-4-yl)-1-(2- fluoroacryloyl)piperazin-2-yl)acetonitrile |
| 63 | 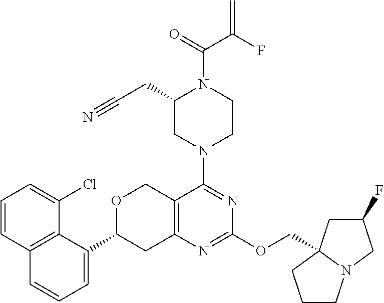 [SMILES: C=C(F)C(=O)N1CCN(c2nc(OC[C@@]34CCCN3C[C@H](F)C4)nc3c2CO[C@@H](c2cccc4cccc(Cl)c24)C3)C[C@@H]1CC#N] | 2-((S)-4-((R)-7-(8-chloronaphthalen-1-yl)-2- (((2R,7aS)-2-fluorotetrahydro-1H-pyrrolizin-7a(5H)- yl)methoxy)-7,8-dihydro-5H-pyrano[4,3-d]pyrimidin- 4-yl)-1-(2-fluoroacryloyl)piperazin-2-yl)acetonitrile |
| 64 | 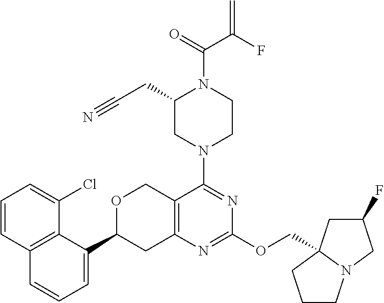 [SMILES: C=C(F)C(=O)N1CCN(c2nc(OC[C@@]34CCCN3C[C@H](F)C4)nc3c2CO[C@H](c2cccc4cccc(Cl)c24)C3)C[C@@H]1CC#N] | 2-((S)-4-((S)-7-(8-chloronaphthalen-1-yl)-2- (((2R,7aS)-2-fluorotetrahydro-1H-pyrrolizin-7a(5H)- yl)methoxy)-7,8-dihydro-5H-pyrano[4,3-d]pyrimidin- 4-yl)-1-(2-fluoroacryloyl)piperazin-2-yl)acetonitrile |
| 65 | 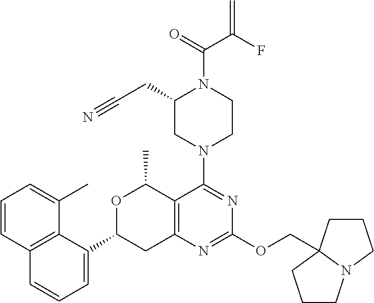 [SMILES: C=C(F)C(=O)N1CCN(c2nc(OCC34CCCN3CCC4)nc3c2[C@@H](C)O[C@@H](c2cccc4cccc(C)c24)C3)C[C@@H]1CC#N] | 2-((S)-1-(2-fluoroacryloyl)-4-((5R,7R)-5-methyl-7-(8- methylnaphthalen-1-yl)-2-((tetrahydro-1H-pyrrolizin- 7a(5H)-yl)methoxy)-7,8-dihydro-5H-pyrano[4,3- d]pyrimidin-4-yl)piperazin-2-yl)acetonitrile |
| 66 | 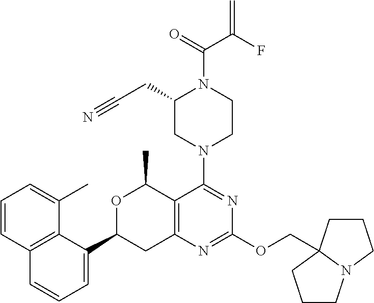 [SMILES: C=C(F)C(=O)N1CCN(c2nc(OCC34CCCN3CCC4)nc3c2[C@H](C)O[C@H](c2cccc4cccc(C)c24)C3)C[C@@H]1CC#N] | 2-((S)-1-(2-fluoroacryloyl)-4-((5S,7S)-5-methyl-7-(8- methylnaphthalen-1-yl)-2-((tetrahydro-1H-pyrrolizin- 7a(5H)-yl)methoxy)-7,8-dihydro-5H-pyrano[4,3- d]pyrimidin-4-yl)piperazin-2-yl)acetonitrile |
| 67 | 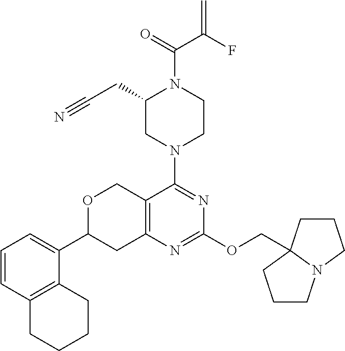 [SMILES: C=C(F)C(=O)N1CCN(c2nc(OCC34CCCN3CCC4)nc3c2COC(c2cccc4c2CCCC4)C3)C[C@@H]1CC#N] | 2-((2S)-1-(2-fluoroacryloyl)-4-(2-((tetrahydro-1H- pyrrolizin-7a(5H)-yl)methoxy)-7-(5,6,7,8- tetrahydronaphthalen-1-yl)-7,8-dihydro-5H- pyrano[4,3-d]pyrimidin-4-yl)piperazin-2- yl)acetonitrile |
| 68 | 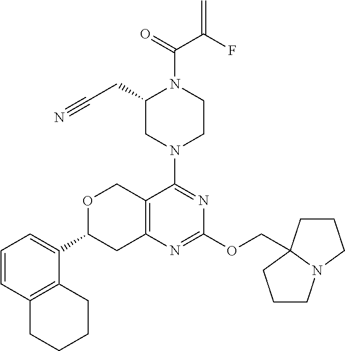 [SMILES: C=C(F)C(=O)N1CCN(c2nc(OCC34CCCN3CCC4)nc3c2CO[C@@H](c2cccc4c2CCCC4)C3)C[C@@H]1CC#N] | 2-((S)-1-(2-fluoroacryloyl)-4-((R)-2-((tetrahydro-1H- pyrrolizin-7a(5H)-yl)methoxy)-7-(5,6,7,8- tetrahydronaphthalen-1-yl)-7,8-dihydro-5H- pyrano[4,3-d]pyrimidin-4-yl)piperazin-2- yl)acetonitrile |
| 69 | 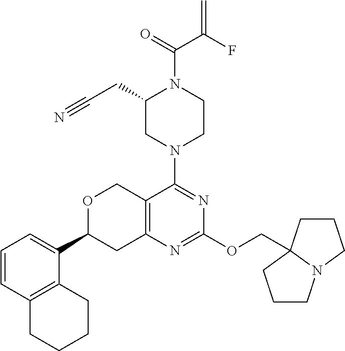 [SMILES: C=C(F)C(=O)N1CCN(c2nc(OCC34CCCN3CCC4)nc3c2CO[C@H](c2cccc4c2CCCC4)C3)C[C@@H]1CC#N] | 2-((S)-1-(2-fluoroacryloyl)-4-((S)-2-((tetrahydro-1H- pyrrolizin-7a(5H)-yl)methoxy)-7-(5,6,7,8- tetrahydronaphthalen-1-yl)-7,8-dihydro-5H- pyrano[4,3-d]pyrimidin-4-yl)piperazin-2- yl)acetonitrile |
| 70 | 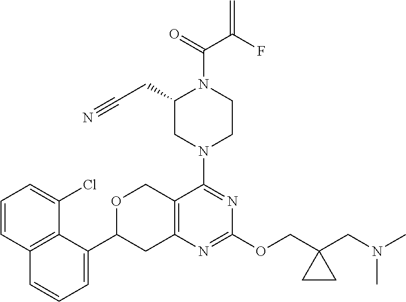 [SMILES: C=C(F)C(=O)N1CCN(c2nc(OCC3(CN(C)C)CC3)nc3c2COC(c2cccc4cccc(Cl)c24)C3)C[C@@H]1CC#N] | 2-((2S)-4-(7-(8-chloronaphthalen-1-yl)-2-((1- ((dimethylamino)methyl)cyclopropyl)methoxy)-7,8- dihydro-5H-pyrano[4,3-d]pyrimidin-4-yl)-1-(2- fluoroacryloyl)piperazin-2-yl)acetonitrile |
| 71 | 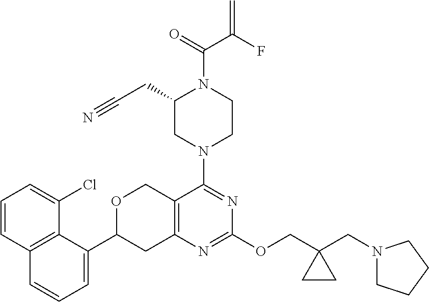 [SMILES: C=C(F)C(=O)N1CCN(c2nc(OCC3(CN4CCCC4)CC3)nc3c2COC(c2cccc4cccc(Cl)c24)C3)C[C@@H]1CC#N] | 2-((2S)-4-(7-(8-chloronaphthalen-1-yl)-2-((1- (pyrrolidin-1-ylmethyl)cyclopropyl)methoxy)-7,8- dihydro-5H-pyrano[4,3-d]pyrimidin-4-yl)-1-(2- fluoroacryloyl)piperazin-2-yl)acetonitrile |
| 72 | 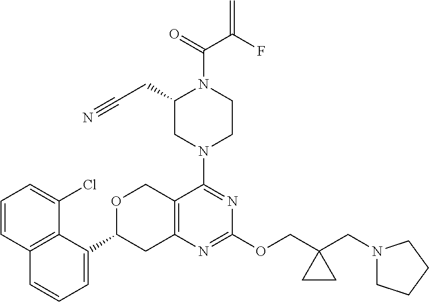 [SMILES: C=C(F)C(=O)N1CCN(c2nc(OCC3(CN4CCCC4)CC3)nc3c2CO[C@@H](c2cccc4cccc(Cl)c24)C3)C[C@@H]1CC#N] | 2-((S)-4-((R)-7-(8-chloronaphthalen-1-yl)-2-((1- (pyrrolidin-1-ylmethyl)cyclopropyl)methoxy)-7,8- dihydro-5H-pyrano[4,3-d]pyrimidin-4-yl)-1-(2- fluoroacryloyl)piperazin-2-yl)acetonitrile |
| 73 | 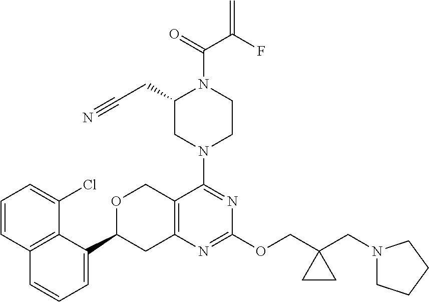 [SMILES: C=C(F)C(=O)N1CCN(c2nc(OCC3(CN4CCCC4)CC3)nc3c2CO[C@H](c2cccc4cccc(Cl)c24)C3)C[C@@H]1CC#N] | 2-((S)-4-((S)-7-(8-chloronaphthalen-1-yl)-2-((1- (pyrrolidin-1-ylmethyl)cyclopropyl)methoxy)-7,8- dihydro-5H-pyrano[4,3-d]pyrimidin-4-yl)-1-(2- fluoroacryloyl)piperazin-2-yl)acetonitrile |
| 74 | 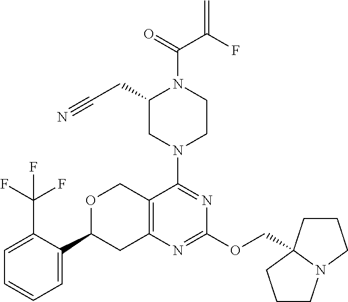 | 2-((S)-1-(2-fluoroacryloyl)-4-((S)-2-((tetrahydro-1H- pyrrolizin-7a(5H)-yl)methoxy)-7-(2- (trifluoromethyl)phenyl)-7,8-dihydro-5H-pyrano[4,3- d]pyrimidin-4-yl)piperazin-2-yl)acetonitrile |
| 75 | 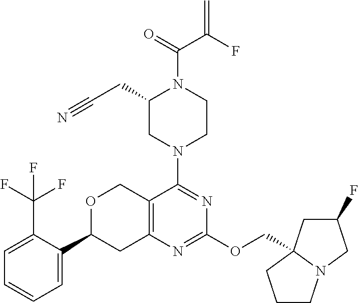 [SMILES: C=C(F)C(=O)N1CCN(c2nc(OC[C@@]34CCCN3C[C@H](F)C4)nc3c2CO[C@H](c2ccccc2C(F)(F)F)C3)C[C@@H]1CC#N] | 2-((S)-1-(2-fluoroacryloyl)-4-((S)-2-(((2R,7aS)-2- fluorotetrahydro-1H-pyrrolizin-7a(5H)-yl)methoxy)-7- (2-(trifluoromethyl)phenyl)-7,8-dihydro-5H- pyrano[4,3-d]pyrimidin-4-yl)piperazin-2- yl)acetonitrile |
| 76 | 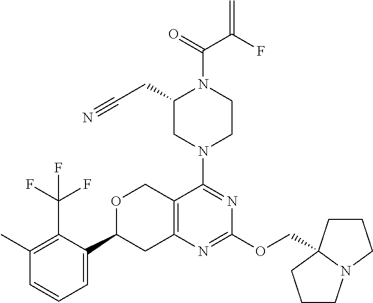 | 2-((S)-1-(2-fluoroacryloyl)-4-((S)-7-(3-methyl-2- (trifluoromethyl)phenyl)-2-((tetrahydro-1H-pyrrolizin- 7a(5H)-yl)methoxy)-7,8-dihydro-5H-pyrano[4,3- d]pyrimidin-4-yl)piperazin-2-yl)acetonitrile |
| 77 | 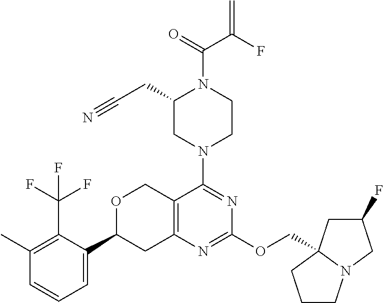 [SMILES: C=C(F)C(=O)N1CCN(c2nc(OC[C@@]34CCCN3C[C@H](F)C4)nc3c2CO[C@H](c2cccc(C)c2C(F)(F)F)C3)C[C@@H]1CC#N] | 2-((S)-1-(2-fluoroacryloyl)-4-((S)-2-(((2R,7aS)-2- fluorotetrahydro-1H-pyrrolizin-7a(5H)-yl)methoxy)-7- (3-methyl-2-(trifluoromethyl)phenyl)-7,8-dihydro-5H- pyrano[4,3-d]pyrimidin-4-yl)piperazin-2- yl)acetonitrile |
| 78 | 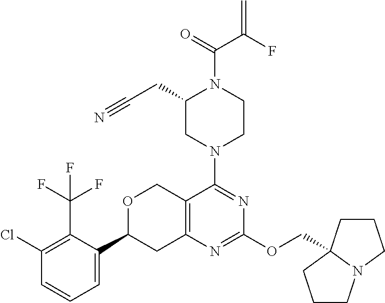 | 2-((S)-4-((S)-7-(3-chloro-2-(trifluoromethyl)phenyl)-2- ((tetrahydro-1H-pyrrolizin-7a(5H)-yl)methoxy)-7,8- dihydro-5H-pyrano[4,3-d]pyrimidin-4-yl)-1-(2- fluoroacryloyl)piperazin-2-yl)acetonitrile |
| 79 | 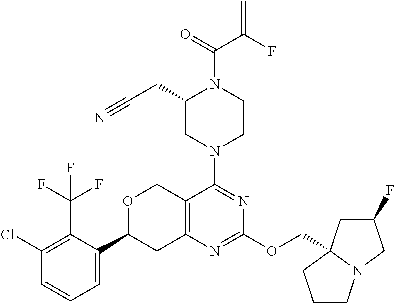 [SMILES: C=C(F)C(=O)N1CCN(c2nc(OC[C@@]34CCCN3C[C@H](F)C4)nc3c2CO[C@H](c2cccc(Cl)c2C(F)(F)F)C3)C[C@@H]1CC#N] | 2-((S)-4-((S)-7-(3-chloro-2-(trifluoromethyl)phenyl)-2- (((2R,7aS)-2-fluorotetrahydro-1H-pyrrolizin-7a(5H)- yl)methoxy)-7,8-dihydro-5H-pyrano[4,3-d]pyrimidin- 4-yl)-1-(2-fluoroacryloyl)piperazin-2-yl)acetonitrile |
| 80 | 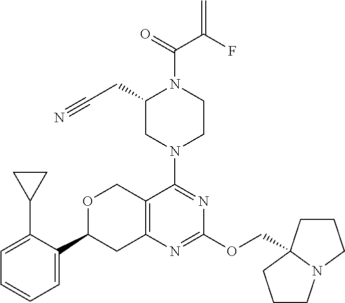 | 2-((S)-4-((S)-7-(2-cyclopropylphenyl)-2-((tetrahydro- 1H-pyrrolizin-7a(5H)-yl)methoxy)-7,8-dihydro-5H- pyrano[4,3-d]pyrimidin-4-yl)-1-(2- fluoroacryloyl)piperazin-2-yl)acetonitrile |
| 81 | 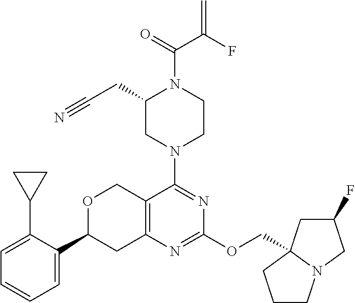 [SMILES: C=C(F)C(=O)N1CCN(c2nc(OC[C@@]34CCCN3C[C@H](F)C4)nc3c2CO[C@H](c2ccccc2C2CC2)C3)C[C@@H]1CC#N] | 2-((S)-4-((S)-7-(2-cyclopropylphenyl)-2-(((2R,7aS)-2- fluorotetrahydro-1H-pyrrolizin-7a(5H)-yl)methoxy)- 7,8-dihydro-5H-pyrano[4,3-d]pyrimidin-4-yl)-1-(2- fluoroacryloyl)piperazin-2-yl)acetonitrile |

### Pharmaceutical Compositions

Pharmaceutical compositions of the present disclosure comprise at least one compound of Formula (1) (e.g. Formula (1A), Formula (1B), Formula (1C)), or a tautomer, stereoisomer or a mixture of stereoisomers, or a pharmaceutically acceptable salt, or hydrate, or deuterated derivative thereof formulated together with one or more pharmaceutically acceptable carriers. These formulations include those suitable for oral, rectal, topical, buccal and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as gelatin and glycerin or sucrose and acacia.

Formulations of the present disclosure suitable for parenteral administration comprise sterile aqueous preparations of at least one compound of Formula (1) (e.g. Formula (1A), Formula (1B), Formula (1C)), or a tautomer, stereoisomer or a mixture of stereoisomers, or a pharmaceutically acceptable salt, or hydrate, or deuterated derivative thereof, which are approximately isotonic with the blood of the intended recipient. These preparations are administered intravenously, although administration may also be effected by means of subcutaneous, intramuscular, or intradermal injection. Such preparations may conveniently be prepared by admixing at least one compound described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula (1) (e.g. Formula (1A), Formula (1B), Formula (1C)), or a tautomer, stereoisomer or a mixture of stereoisomers, or a pharmaceutically acceptable salt, or hydrate, or deuterated derivative thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 pg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following Table for Equivalent Surface Area Dosage Factors).

TABLE 2
Equivalent Surface Area Dosage Factors.

| | To: | | | | |
|---|---|---|---|---|---|
| | Mouse | Rat | Monkey | Dog | Human |
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

### Methods of Treatment

In some embodiments, a compound of Formula (1) (e.g. Formula (1A), Formula (1B), Formula (1C)), or a tautomer, stereoisomer or a mixture of stereoisomers, or a pharmaceutically acceptable salt, or hydrate, or deuterated derivative thereof, is administered to treat cancer in a subject in need thereof. In some embodiments, the cancer is chosen from breast cancer, lung cancer, pancreatic cancer, colorectal cancer, gall bladder cancer, thyroid cancer, bile duct cancer, ovarian cancer, endometrial cancer, prostate cancer, and esophageal cancer. In some embodiments, the cancer is breast cancer. In some embodiments, the cancer is lung cancer. In some embodiments, the cancer is pancreatic cancer. In some embodiments, the cancer is colorectal cancer. In some embodiments, the cancer is gall bladder cancer. In some embodiments, the cancer is thyroid cancer. In some embodiments, the cancer is bile duct cancer. In some embodiments, the cancer is ovarian cancer. In some embodiments, the cancer is endometrial cancer. In some embodiments, the cancer is prostate cancer. In some embodiments, the cancer is esophageal cancer. In some embodiments, the therapeutic treatment is for the treatment of KRAS G12-associated diseases and conditions.

In some embodiments, a compound of Formula (1) (e.g. Formula (1A), Formula (1B), Formula (1C)), or a tautomer, stereoisomer or a mixture of stereoisomers, or a pharmaceutically acceptable salt, or hydrate, or deuterated derivative thereof, is administered as a pharmaceutical composition.

In some embodiments, the invention provides for methods for inhibiting KRas G12C activity in a cell, comprising contacting the cell in which inhibition of KRas G12C activity is desired with an effective amount of a compound of Formula (1) (e.g. Formula (1A), Formula (1B), Formula (1C)), pharmaceutically acceptable salts thereof or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” a KRas G12C with a compound provided herein includes the administration of a compound provided herein to an individual or patient, such as a human, having KRas G12C, as well as, for example, introducing a compound provided herein into a sample containing a cellular or purified preparation containing the KRas G12C.

In one embodiment, a cell in which inhibition of KRas G12C activity is desired is contacted with an effective amount of a compound of Formula (1) (e.g. Formula (1A), Formula (1B), Formula (1C)) to negatively modulate the activity of KRas G12C. In some embodiments, a therapeutically effective amount of pharmaceutically acceptable salt or pharmaceutical compositions containing the compound of Formula (1) (e.g. Formula (1A), Formula (1B), Formula (1C)) may be used.

By negatively modulating the activity of KRas G12C, the methods described herein are designed to inhibit undesired cellular proliferation resulting from enhanced KRas G12C activity within the cell. The cells may be contacted in a single dose or multiple doses in accordance with a particular treatment regimen to effect the desired negative modulation of KRas G120.

The concentration and route of administration to the patient will vary depending on the cancer to be treated.

In one embodiment, a compound of Formula (1) (e.g. Formula (1A), Formula (1B), Formula (1C)), or a tautomer, stereoisomer or a mixture of stereoisomers, or a pharmaceutically acceptable salt, or hydrate, or deuterated derivative thereof, is administered in combination with another therapeutic agent, e.g., chemotherapy, or used in combination with other treatments, such as radiation or surgical intervention, either as an adjuvant prior to surgery or post-operatively.

In some embodiments, the subject has been previously treated with an anti-cancer agent. In some embodiments, a compound of Formula (1) (e.g. Formula (1A), Formula (1B), Formula (1C)), or a pharmaceutically acceptable salt or solvate thereof, as defined herein, may be administered to a subject in combination with an anti-cancer agent. In some embodiments, the anti-cancer agent is BRAF inhibitor. In some embodiments, the anti-cancer agent is MEK inhibitor. In some embodiments, the anti-cancer agent is ERK inhibitor. In some embodiments, the anti-cancer agent is SHP2 inhibitor. In some embodiments, the anti-cancer agent is SOS1 inhibitor. In some embodiments, the anti-cancer agent is PI3K inhibitor. In some embodiments, the anti-cancer agent is AKT inhibitor. In some embodiments, the anti-cancer agent is PD1/PDL1 inhibitor. In some embodiments, the anti-cancer agent is NRF2 inhibitor. In some embodiments, the anti-cancer agent is AMPK activator. In some embodiments, the anti-cancer agent is WNT inhibitor. In some embodiments, the anti-cancer agent is an mTOR inhibitor. In some embodiments, the anti-cancer agent is an Insulin-like Growth Factor 1 receptor (IGF-1R) inhibitor.

In some embodiments, the anti-cancer agent is an epidermal growth factor receptor (EGFR) inhibitor. In some embodiments, the EGFR inhibitor is cetuximab. In some embodiments, the EGFR inhibitor is afatinib.

Also provided herein is a compound of Formula (1) (e.g. Formula (1A), Formula (1B), Formula (10)), or a pharmaceutically acceptable salt or solvate thereof, or a pharmaceutical composition thereof as defined herein for use in therapy.

Also provided herein is a compound of Formula (1) (e.g. Formula (1A), Formula (1B), Formula (1C)), or a pharmaceutically acceptable salt or solvate thereof or a pharmaceutical composition thereof as defined herein for use in the treatment of cancer.

Also provided herein is a compound of Formula (1) (e.g. Formula (1A), Formula (1B), Formula (1C)), or a pharmaceutically acceptable salt or solvate thereof for use in the inhibition of KRas G120.

Also provided herein is a compound of Formula (1) (e.g. Formula (1A), Formula (1B), Formula (1C)), or a pharmaceutically acceptable salt or solvate thereof or a pharmaceutical composition thereof as defined herein, for use in the treatment of a KRas G12C-associated disease or disorder.

Also provided herein is the use of a compound of Formula (1) (e.g. Formula (1A), Formula (1B), Formula (1C)), or a pharmaceutically acceptable salt or solvate thereof, as defined herein in the manufacture of a medicament for the treatment of cancer.

Also provided herein is a use of a compound of Formula (1) (e.g. Formula (1A), Formula (1B), Formula (1C)), or a pharmaceutically acceptable salt or solvate thereof, as defined herein in the manufacture of a medicament for the inhibition of activity of KRas G12C.

Also provided herein is the use of a compound of Formula (1) (e.g. Formula (1A), Formula (1B), Formula (1C)), or a pharmaceutically acceptable salt or solvate thereof, as defined herein, in the manufacture of a medicament for the treatment of a KRas G120-associated disease or disorder.

### EXAMPLES

The examples and preparations provided below further illustrate and exemplify the compounds as disclosed herein and methods of preparing such compounds. It is to be understood that the scope of the present disclosure is not limited in any way by the scope of the following examples and preparations.

The chemical entities described herein can be synthesized according to one or more illustrative schemes herein and/or techniques well known in the art. Unless specified to the contrary, the reactions described herein take place at atmospheric pressure, generally within a temperature range from about −78° C. to about 200° C. Further, except as otherwise specified, reaction times and conditions are intended to be approximate, e.g., taking place at about atmospheric pressure within a temperature range of about −78° C. to about 200° C. over a period that can be, for example, about 1 to about 24 hours; reactions left to run overnight in some embodiments can average a period of about 16 hours.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3^{rd} Ed., 1990 New York: Plenum Press; Mundy et al., Name Reaction and Reagents in Organic Synthesis, 2^{nd} Ed., 2005 Hoboken, N.J.: J. Wiley & Sons. Specific illustrations of suitable separation and isolation procedures are given by reference to the examples hereinbelow. However, other equivalent separation or isolation procedures can also be used.

In all of the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3^{rd} Ed., John Wiley & Sons). These groups may be removed at a convenient stage of the compound synthesis using methods that are readily apparent to those skilled in the art.

When desired, the (R)- and (S)-isomers of the nonlimiting exemplary compounds, if present, can be resolved by methods known to those skilled in the art, for example, by formation of diastereoisomeric salts or complexes which can be separated, e.g., by crystallization; via formation of diastereoisomeric derivatives which can be separated, e.g., by crystallization, gas-liquid or liquid chromatography; selective reaction of one enantiomer with an enantiomer-specific reagent, e.g., enzymatic oxidation or reduction, followed by separation of the modified and unmodified enantiomers; or gas-liquid or liquid chromatography in a chiral environment, e.g., on a chiral support, such as silica with a bound chiral ligand or in the presence of a chiral solvent. Alternatively, a specific enantiomer can be synthesized by asymmetric synthesis using optically active reagents, substrates, catalysts or solvents, or by converting one enantiomer to the other by asymmetric transformation.

The compounds described herein can be optionally contacted with a pharmaceutically acceptable acid to form the corresponding acid addition salts. Also, the compounds described herein can be optionally contacted with a pharmaceutically acceptable base to form the corresponding basic addition salts.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Millipore Sigma or can be readily prepared by those skilled in the art using commonly employed synthetic methodology.

The discussion below is offered to illustrate certain of the diverse methods available for use in making the disclosed compounds and is not intended to limit the scope of reactions or reaction sequences that can be used in preparing the compounds provided herein. The skilled artisan will understand that standard atom valencies apply to all compounds disclosed herein in genus or named compound for unless otherwise specified.

The following abbreviations have the definitions set forth below:
1. ACN: Acetonitrile
2. DCE: Dichloroethane
3. DCM: Dichloromethane
4. DIEA: Diisopropylethylamine
5. DMA: N,N-dimethylacetamide
6. EA: Ethyl acetate
7. HPLC: High pressure liquid chromatography
8. LC/MS: Liquid chromatography/Mass spectroscopy
9. NMR: Nuclear magnetic resonance
10.PE: Petroleum ether
11. Pd₂(dba)₃: Tris(dibenzylideneacetone)dipalladium (0)
12. RuPhos: 2-Dicyclohexylphosphino-2,6′-diisopropoxybiphenyl
13. RuPhos-Pd-G3: (2-Dicyclohexylphosphino-2,6′-diisopropoxy-1,1′-biphenyl)[2-(2′-amino-1,1′-biphenyl)]palladium(II) methanesulfonate
14. TEA: Triethylamine
15. TLC: Thin layer chromatography
16. TR-FRET: Time-resolved fluorescence energy transfer
17. XPhos: 2-Dicyclohexylphosphino-2,4,6′-triisopropylbiphenyl
18. XPhos-Pd-G3: (2-Dicyclohexylphosphino-2,4,6′-triisopropyl-1,1′-biphenyl)[2-(2′-amino-1,1¹-biphenyl)]palladium(II) methanesulfonate

General Synthetic Schemes

HPLC spectra for all compounds were acquired using an Agilent 1200 Series system with DAD detector. Chromatography was performed on a 2.1×150 mm Zorbax 300SB-C_{18 5} μm column with water containing 0.1% formic acid as solvent A and acetonitrile containing 0.1% formic acid as solvent B at a flow rate of 0.4 mL/min. The gradient program was as follows: 1% B (0-1 min), 1-99% B (1-4 min), and 99% B (4-8 min). High-resolution mass spectra (HRMS) data were acquired in positive ion mode using an Agilent G1969A API-TOF with an electrospray ionization (ESI) source. Nuclear Magnetic Resonance (NMR) spectra were acquired on a Bruker spectrometer with 600 MHz or 400 MHz for proton (¹H NMR) and 150 MHz for carbon (¹³0 NMR); chemical shifts are reported in (δ). Preparative HPLC was performed on Agilent Prep 1200 series with UV detector set to 254 nm and 220 nm. Samples were injected onto a Phenomenex Luna 75×30 mm, 5 μm, C₁₈ column at room temperature. The flow rate was 40 mL/min. A linear gradient was used with 10% (or 50%) of MeOH (A) in H₂O (with 0.1% TFA) (B) to 100% of MeOH (A). HPLC was used to establish the purity of target compounds. All final compounds were determined to be >95% purity when analyzed according to the HPLC methods described above.

Compounds of Formula (1) (e.g. Formula (1A), Formula (1 B), Formula (1C); see compounds in Table 1) can be prepared according to the following schemes. The following schemes represent the general methods used in preparing these compounds. However, the synthesis of these compounds is not limited to these representative methods, as they can also be prepared by various other methods those skilled in the art of synthetic chemistry, for example, in a stepwise or modular fashion.

### Scheme 1: General method for the preparation of 2,4,7-tri-substituted dihydropyranopyrimidine (Formula (1B))

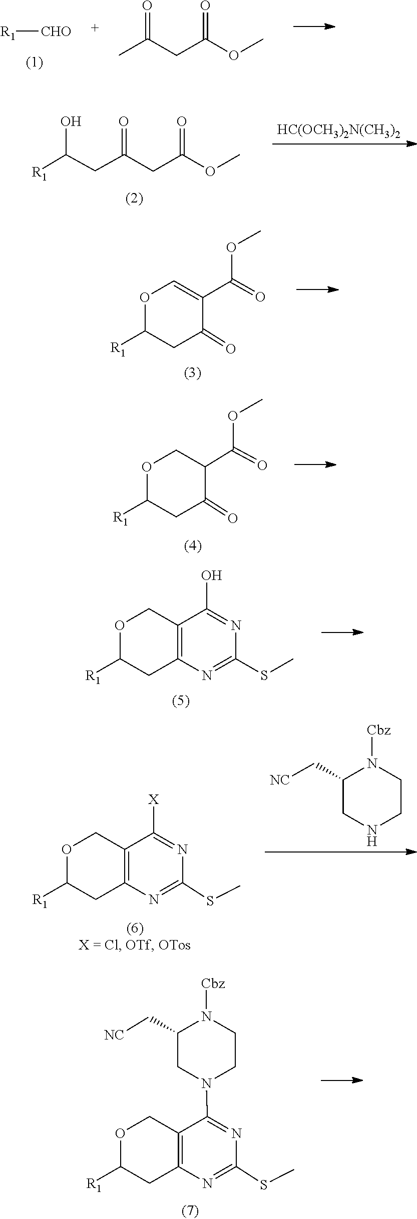

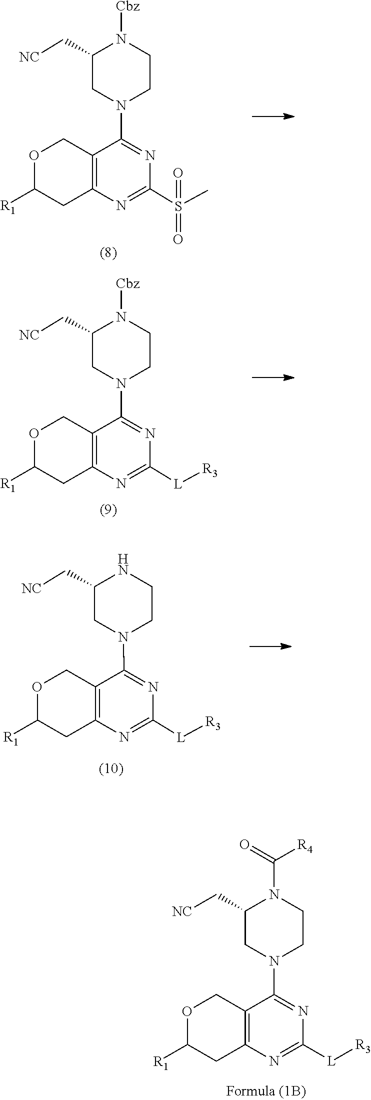

Compounds of Formula (1 B) represent a tri-substituted dihydropyranopyrimidine, which can be prepared according to general Scheme 1. The substituted aromatic aldehyde (1) can react with acetoacetate in the presence of a base such as NaH or LHMDS to provide 5-hydroxy-3-oxo-pentanoate compound (2). Compound (2) can react with N,N-dimethylformamide dimethyl acetal followed by cyclization promoted by Lewis acid such as BF₃.OEt₂to provide dihydropyran-4-one compound (3) (Organic & Biomolecular Chemistry (2016), 14(28), 6840-6852). Compound (3) can be reduced with a reducing agent such as lithium tri-sec-butylborohydride to give a keto ester compound (4). The pyrimidine ring in compound (5) can be formed through cyclization of compound (4) with methylisothiourea. The hydroxyl group in compound (5) can be converted to a leaving group such as chloro, triflate or tosylate using reagents known in the literature for this kind of functional group transformation to provide compound (6). Intermediate (6) can react with benzyl (S)-2-(cyanomethyl)piperazine-1-carboxylate in a polar solvent such as DMF in the presence of a base such as DIEA to provide compound (7). The methylthio group in compound (7) can be oxidized to sulfone intermediate (8) by an oxidant such as mCPBA. The methylsulf one group in intermediate (8) can be substituted by alcohol, thioalcohol or amine (HL-R₃) to give compound (9), where L can be a bond, 0, NH, S or alkylamino group. The Cbz protecting group of compound (9) can be removed by hydrogenolysis with Pd/C, followed by acrylamide formation to provide the desired compounds of Formula (1B).

4-Oxo-3,4-dihydro-2H-pyran-5-carboxylate intermediate 3 of Scheme 1 can also be prepared by Diels-Alder cyclization between ethyl 2-(ethoxymethylene)-3-((trimethylsilyl)oxy)but-3-enoate and aryl aldehyde. In the presence of Lewis acid and chiral ligand, the cyclization reaction can provide one of the enantiomeric isomers with high enantioselectivity.

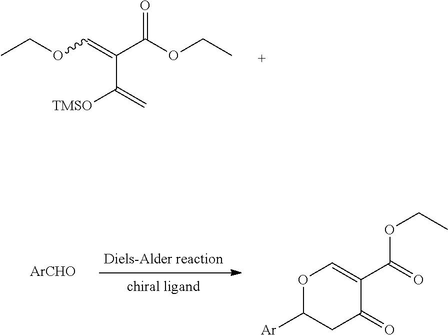

### Scheme 2: General method for the preparation of 2,4,5,7-tetra-substituted dihydropyranopyrimidine (Formula (1C))

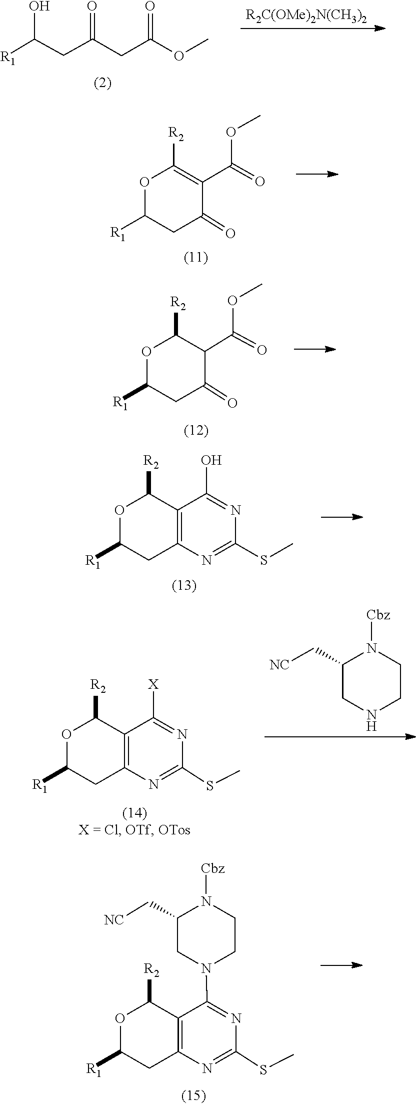

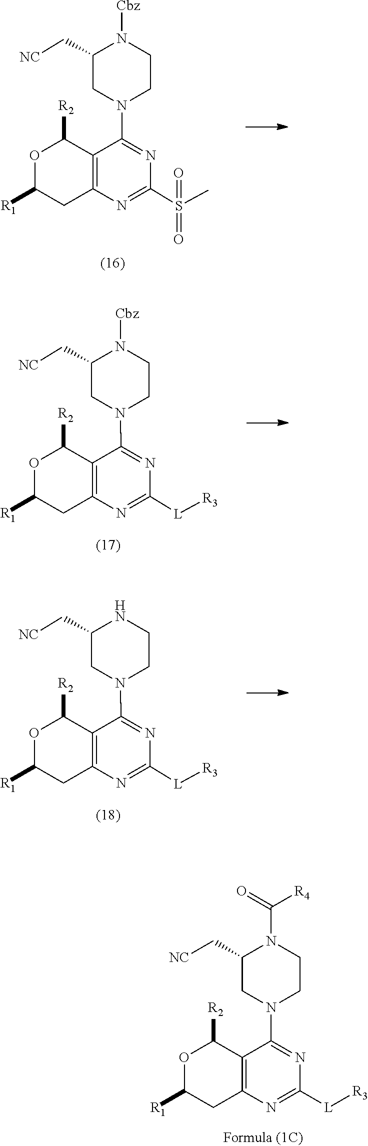

Compounds of Formula (1C) represent a tetra-substituted dihydropyranopyrimidine, which can be prepared according to general scheme 2. Intermediate (2) from Scheme 1 can react with 1,1-dimethoxy-N,N-dimethylalkyl-1-amine to form the dihydropyran-4-one (11). Reduction of compound (11) with L-selectride can provide cis-2,6-di-substituted intermediate (12) following literature procedure (Organic & Biomolecular Chemistry (2016), 14(28), 6840-6852). Conversion of compound (12) to the desired final product (Formula (1C)) can follow the same procedure as described in Scheme 1.

### Scheme 3: Alternative Method for the Preparation of Substituted Dihydropyranopyrimidine

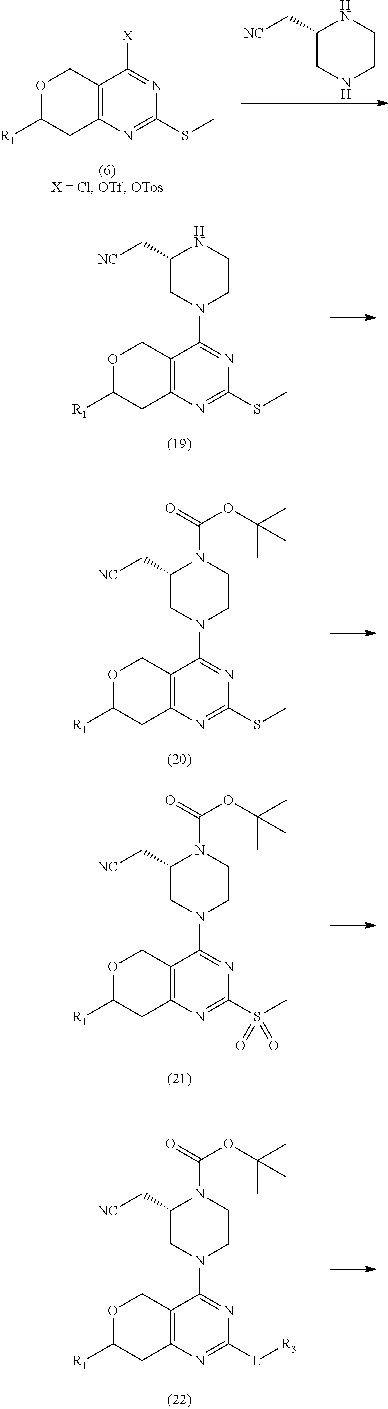

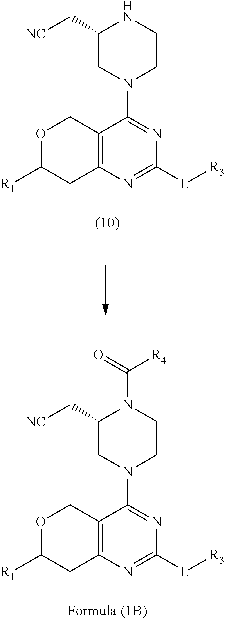

Alternatively, intermediate (6) from Scheme 1 can react with (S)-2-(piperazin-2-yl)acetonitrile to provide compound (19). The piperazine NH in compound (19) can be protected to give intermediate (20). Oxidation of intermediate (20) followed by the displacement of methylsufone with HL-R₃ in the presence of base can lead to the formation of compound (22), where L can be a bond, O, S, NH or alkylamino group. The Boc group in compound (22) can be cleaved under acidic condition and the corresponding product (10) can be acylated to form the desired compounds of Formula (1B).

### Preparation of Example Compounds

### Example 1

### Synthesis of 2-((2S)-1-acryloyl-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Compound 3)

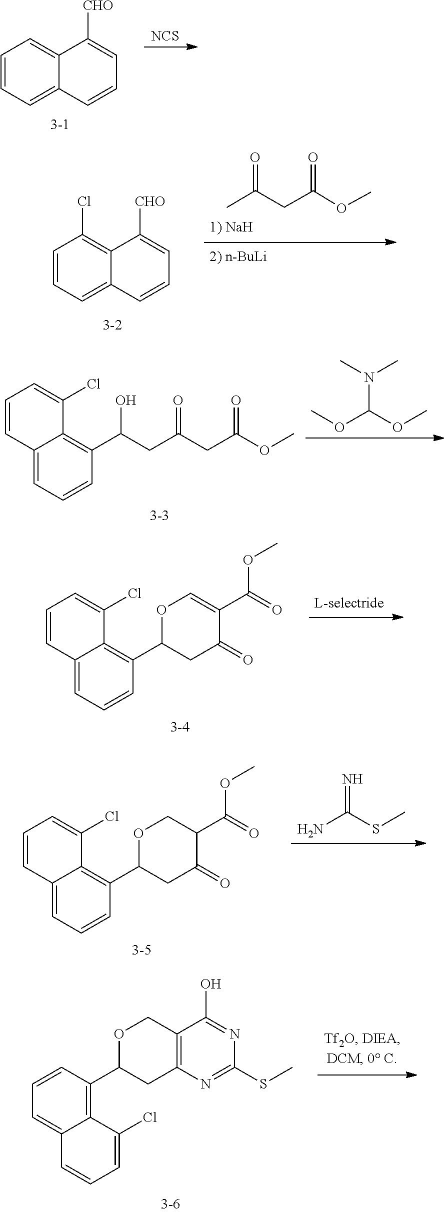

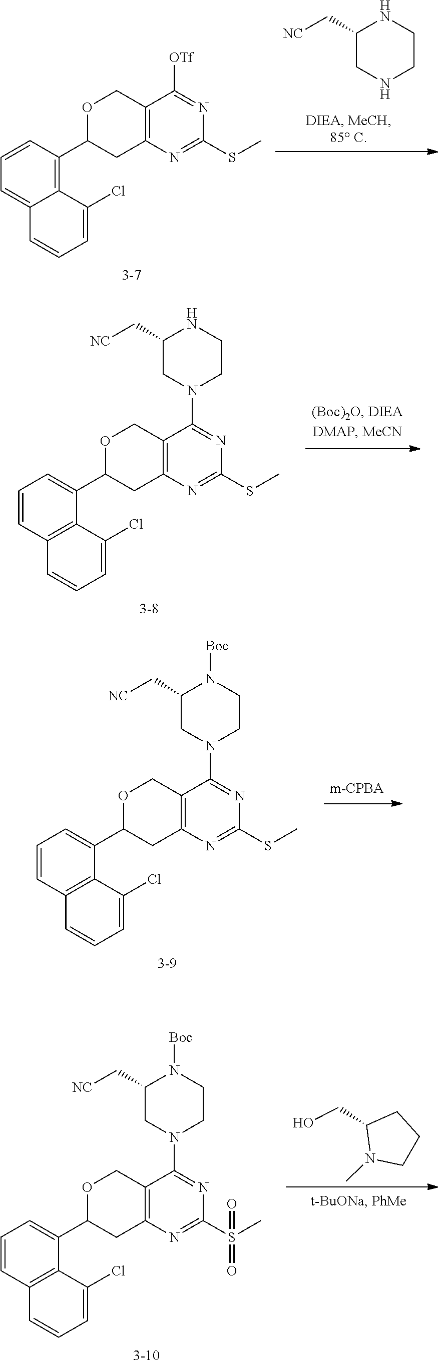

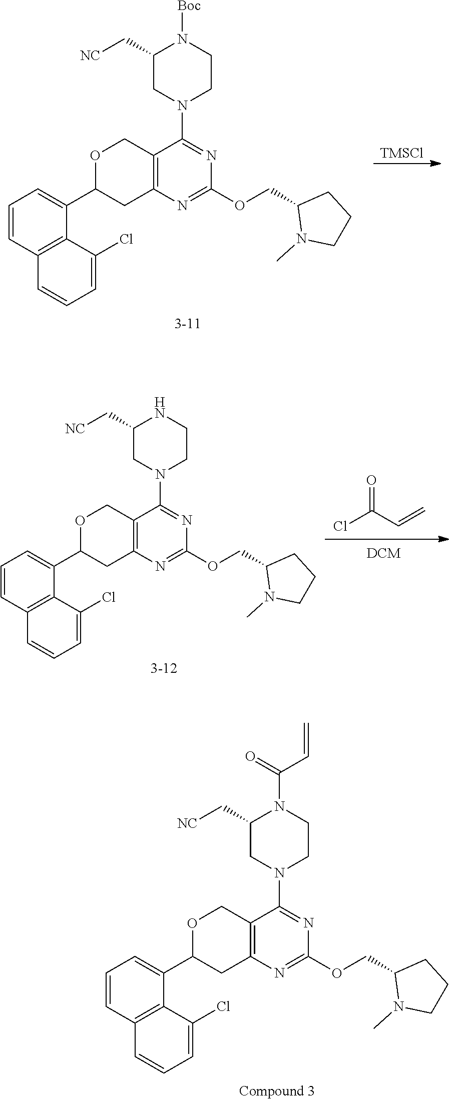

### Step 1: Preparation of 8-chloro-1-naphthaldehyde (3-2)

To a solution of naphthalene-1-carbaldehyde (3-1) (20 g, 128.1 mmol) in DCE (300 mL) at 0° C. was added NCS (20.4 g, 153.7 mmol), Pd(OAc)₂ (2.88 g, 12.8 mmol) and TfOH (19.2 g, 128.1 mmol). Then the mixture was stirred at 80° C. for 24 h. The resulting solution was quenched with ice/water, extracted with DCM (100 mL×2). The combined organic layers were washed with brine, dried over Na₂SO₄, filtered and the filtrate was concentrated and purified by Combi-Flash (eluted by EA/PE=0-10%) to give 8-chloronaphthalene-1-carbaldehyde (16 g, 59%) as a yellow solid. LC/MS: 190.9[M+H]+.

### Step 2: Preparation of methyl 5-(8-chloronaphthalen-1-yl)-5-hydroxy-3-oxopentanoate (3-3)

To a solution of methyl 3-oxobutanoate (3.1 g, 26.2 mmol) in THF (80 mL) was added NaH (1.3 g, 31.5 mmol, 60%). After stirring at rt for 30 min, the mixture was cooled to −10° C., n-BuLi (13.1 mL, 31.4 mmol, 2.4 M) was added dropwise. The reaction was stirred for 1 h, and 8-chloro-1-naphthaldehyde (5 g, 26.2 mmol) was added. The resulting solution was stirred overnight. The mixture was poured into aqueous NH₄Cl solution, extracted with EA (200 mL×3). The combined organic layer was dried over Na₂SO₄, filtered and the filtrate was concentrated. The crude product was purified with Combi-Flash (EA/PE=1/2) to give methyl 5-(8-chloronaphthalen-1-yl)-5-hydroxy-3-oxopentanoate (5.3 g, 59.3%) as a yellow solid. LC/MS: 328.8[M+Na]+.

### Step 3: Preparation of methyl 2-(8-chloronaphthalen-1-yl)-4-oxo-3,4-dihydro-2H-pyran-5-carboxylate (3-4)

A mixture of methyl 5-(8-chloronaphthalen-1-yl)-5-hydroxy-3-oxopentanoate (6 g, 22 mmol) and (dimethoxymethyl)dimethylamine (2.9 g, 24.2 mmol) in DCM (200 mL) was stirred at room temperature for 1 h. Then BF₃.Et₂O (3.4 g, 24.2 mmol) was added, the mixture was stirred at rt for 2 h. DCM (300 mL) was added into the mixture, and the mixture was washed with aqueous NaHCO₃ solution. The organic layer was dried over Na₂SO₄, filtered and the filtrate was concentrated to give methyl 2-(8-chloronaphthalen-1-yl)-4-oxo-3,4-dihydro-2H-pyran-5-carboxylate (6.2 g, 69%) as a yellow oil. The product was used in next step without further purification. LC/MS: 316.8[M+H]+.

### Step 4: Preparation of methyl 6-(8-chloronaphthalen-1-yl)-4-oxotetrahydro-2H-pyran-3-carboxylate (3-5)

To a solution of methyl 2-(8-chloronaphthalen-1-yl)-4-oxo-3,4-dihydro-2H-pyran-5-carboxylate (6.2 g, 22 mmol) in THF (200 mL) was added L-selectride (24.2 mL, 1 M) at −78° C. under N₂ atmosphere. The mixture was stirred at this temperature for 1 h, then poured into cooled aqueous NH₄Cl solution, extracted with EA (200 mL×3). The combined organic layers were dried over Na₂SO₄, filtered and the filtrate was concentrated to give 6-(8-chloronaphthalen-1-yl)-4-oxotetrahydro-2H-pyran-3-carboxylate (4.6 g, 68%) as a yellow oil. The product was used in next step without further purification. LC/MS: 318.8[M+H]+.

### Step 5: Preparation of 7-(8-chloronaphthalen-1-yl)-2-(methylthio)-7,8-dihydro-5H-pyrano[4,3-d]pyrimidin-4-ol (3-6)

To a solution of methyl 6-(8-chloronaphthalen-1-yl)-4-oxooxane-3-carboxylate (4.6 g, 14.4 mmol) and (methylsulfanyl)methanimidamide (1.97 g, 14.4 mmol) in EtOH/H20=5:1 (180 mL) was added sodium carbonate (12.2 g, 115.5 mmol) at 25° C. under N2 atmosphere. The mixture was stirred at 25° C. for 17 hrs. Then the mixture was warmed to 50° C. and stirred for 3 h. The mixture was filtered and the filtrate was concentrated. The crude residue was diluted with H₂O (50 mL), adjusted to pH=5 with AcOH, extracted with DCM (150 mL×2). The combined organic layers were dried over Na₂SO₄, filtered and the filtrate was concentrated. The crude product was washed with DCM/PE (1:3) and DCM/MTBE (1:15), then filtered. The solid was concentrated to give 7-(8-chloronaphthalen-1-yl)-2-(methylthio)-7,8-dihydro-5H-pyrano[4,3-d]pyrimidin-4-ol (1 g, 17.3%) as a yellow solid. LC/MS: 358.8 [M+H]+.

### Step 6: Preparation of 7-(8-chloronaphthalen-1-yl)-2-(methylthio)-7,8-dihydro-5H-pyrano[4,3-d]pyrimidin-4-yl trifluoromethanesulfonate (3-7)

To a solution of 7-(8-chloronaphthalen-1-yl)-2-(methylsulfanyl)-5H,7H,8H-pyrano[4,3-d]pyrimidin-4-ol (1 g, 3.0 mmol) in DCM (30 mL) was added DIEA (792.3 mg, 6.1 mmol) and Tf₂O (1.56 g, 5.5 mmol) at 0° C. under N₂ atmosphere. The mixture was stirred at 0° C. for 2 h. Water (20 mL) was added, and the mixture was extracted with DCM (20 mL×2). The combined organic layers were dried over Na₂SO₄, filtered and the filtrate was concentrated to give 7-(8-chloronaphthalen-1-yl)-2-(methylthio)-7,8-dihydro-5H-pyrano[4,3-d]pyrimidin-4-yl trifluoromethanesulfonate (1.5 g, crude) as a brown solid. The product was used in next step without further purification. LC/MS:490.5 [M+H]+.

### Step 7: Preparation of 2-((2S)-4-(7-(8-chloronaphthalen-1-yl)-2-(methylthio)-7,8-dihydro-5H-pyrano[4,3-d]pyrimidin-4-yl)piperazin-2-yl)acetonitrile (3-8)

To a mixture of 7-(8-chloronaphthalen-1-yl)-2-methoxy-5H,7H,8H-pyrano[4,3-d]pyrimidin-4-yl trifluoromethanesulfonate (1.5 g, crude) and 2-[(2S)-piperazin-2-yl]acetonitrile (514 mg, 4.1 mmol) in MeCN (30 mL) was added DIEA (1.3 g, 9.5 mmol) slowly. The reaction was stirred for 2 h at 80° C. Then the reaction was concentrated to give 2-((2S)-4-(7-(8-chloronaphthalen-1-yl)-2-(methylthio)-7,8-dihydro-5H-pyrano[4,3-d]pyrimidin-4-yl)piperazin-2-yl)acetonitrile (1.8 g, crude) as a brown solid, which was used in next step without further purification. LC/MS:465.7 [M+H]+.

### Step 8: Preparation of tert-butyl (2S)-4-(7-(8-chloronaphthalen-1-yl)-2-(methylthio)-7,8-dihydro-5H-pyrano[4,3-d]pyrimidin-4-yl)-2-(cyanomethyl)piperazine-1-carboxylate(3-9)

To a mixture of 2-((2S)-4-(7-(8-chloronaphthalen-1-yl)-2-(methylthio)-7,8-dihydro-5H-pyrano[4,3-d]pyrimidin-4-yl)piperazin-2-yl)acetonitrile (1.8 g, crude), (Boc)₂O (7.4 g, 33.8 mmol) and DIEA (4.4 g, 33.8 mmol) in MeCN (30 mL) was added DMAP (412.5 mg, 3.3 mmol). The reaction was stirred for 60 hrs at 25° C. Then the reaction was concentrated and diluted with EA (50 mL). The organic layer was washed with sat. NaHCO₃ solution and brine. The organic layer was dried over Na₂SO₄, filtered and the filtrate was concentrated. The crude product was purified with flash column chromatography (PE/EA=2/1) to provide tert-butyl (2S)-4-(7-(8-chloronaphthalen-1-yl)-2-(methylthio)-7,8-dihydro-5H-pyrano[4,3-d]pyrimidin-4-yl)-2-(cyanomethyl)piperazine-1-carboxylate (390 mg, 16.4% for three steps) as a yellow solid. LC/MS:565.6[M+H]+.

### Step 9: Preparation of tert-butyl (2S)-4-(7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate (3-10)

To a mixture of tert-butyl (2S)-4-[7-(8-chloronaphthalen-1-yl)-2-(methylsulfanyl)-5H,7H,8H-pyrano[4,3-d]pyrimidin-4-yl]-2-(cyanomethyl)piperazine-1-carboxylate (390 mg, 0.69 mmol) in DCM (15 mL) was added 3-chloroperoxybenzoic acid (419.6 mg, 2.1 mmol) slowly. The reaction was stirred for 3 h at 25° C. Then the reaction was diluted with DCM (50 mL). The organic layer was washed with sat. Na₂S₂O₃ solution, sat. NaHCO₃ solution and brine. The organic layer was dried over Na₂SO₄, filtered and the filtrate was concentrated. The crude product was purified with flash column chromatography (MeOH/DCM=0-5%) to give tert-butyl (2S)-4-(7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate (340 mg, 74.6%) as a yellow solid. LC/MS:597.5[M+H]+.

### Step 10: Preparation of tert-butyl (2S)-4-(7-(8-chloronaphthalen-1-yl)-2-(((S)-1-methylpyrrolidin-2₁1)methoxy)-7,8-dihydro-5H-pyrano[4,3-d]pyrimidin-4-yl)-2-(cyanomethyl) piperazine-1-carboxylate (3-11)

To a mixture of tert-butyl (2S)-4-[7-(8-chloronaphthalen-1-yl)-2-methanesulfonyl-5H,7H,8H-pyrano[4,3-d]pyrimidin-4-yl]-2-(cyanomethyl)piperazin-1-yl formate (340 mg, 0.57 mmol) and [(2S)-1-methylpyrrolidin-2-yl]methanol (130.7 mg, 1.14 mmol) in toluene (10 mL) was added sodium tert-butoxide (81.8 mg, 0.85 mmol) slowly. The reaction was stirred for 2 h at 0° C. The reaction was quenched by sat. NH₄Cl solution and extracted with EA (10 mL×3). The organic layer was washed with brine and dried over Na₂SO₄, filtered and the filtrate was concentrated. The crude residue was purified with Combi-Flash (MeOH/DCM=1/10) to give tert-butyl (2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-2-(cyanomethyl) piperazine-1-carboxylate (260 mg, 65.1%) as a yellow solid. LC/MS:632.6[M+H]+.

### Step 11: Preparation of 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (3-12)

To a mixture of tert-butyl (2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-2-(cyanomethyl) piperazine-1-carboxylate (260 mg, 0.41 mmol) in 2,2,2-Trifluoroethanol (5 mL) was added TMSCI (133.6 mg, 1.2 mmol) slowly. The reaction was stirred for 4 h at 20° C. Then the reaction was concentrated to give 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (200 mg, 82.3%) as a yellow solid. LC/MS: 532.7 [M+H]+.

### Step 12: Preparation of 2-((2S)-1-acryloyl-4-(7-(8-chloronaphthalen-1-yl)-2-(((S)-1-methylpyrrolidin-2₁1)methoxy)-7,8-dihydro-5H-pyrano[4,3-d]pyrimidin-4-yl)piperazin-2-yl)acetonitrile (Compound 3)

To a mixture of 2-[(2S)-4-[7-(8-chloronaphthalen-1-yl)-2-{[(2S)-1-methylpyrrolidin-2-yl]methoxy}-5H,7H,8H-pyrano[4,3-d]pyrimidin-4-yl]piperazin-2-yl]acetonitrile (160 mg, 0.30 mmol) and DIEA (77.6 mg, 0.60 mmol) in DCM (6 mL) was added prop-2-enoyl chloride (27.2 mg, 0.30 mmol) slowly. The reaction was stirred for 1 h at 0° C. Then the reaction was concentrated. The crude residue was purified by Prep-HPLC using a gradient of H₂O (0.1% TFA)/ACN from 70% to 40%, and suitable fractions were pooled and lyophilized to give 2-((2S)-1-acryloyl-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (96 mg, 42.7%) as a white solid. LC/MS:586.6[M+H]+; 1H NMR (400 MHz, MeOD) δ 8.03-8.00 (m, 1H), 7.92-7.90 (m, 2H), 7.66 (d, J=7.5 Hz, 1H), 7.58 (t, J=7.8 Hz, 1H), 7.42 (t, J=7.8 Hz, 1H), 6.91-6.77 (m, 1H), 6.57-6.47 (dd, J=12.0, 4.0 Hz, 1H), 6.30 (d, J=12.0 Hz, 1H), 5.83 (d, J=12.0 Hz, 1H), 5.14 (d, J=16.0 Hz, 2H), 4.91-4.80 (m,2H), 4.78 (d, J=16.0 Hz, 1H), 4.51-4.57 (m, 1H), 3.99-4.18 (m, 3H), 3.49-3.87 (m,4H), 3.30-3.17 (m, 2H), 3.05 (s, 3H), 2.90 (br, 2H), 2.77-2.73 (m, 1H), 2.37-2.34 (br, 1H), 1.99-2.19 (m, 3H).

### Examples 2 and 3

### Synthesis of 2-((S)-1-acryloyl-4-((R)-7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Compound 6) and Synthesis of 2-((S)-1-acryloyl-4-((S)-7-(8-chloronaphthalen-1-yl)-2-(((S)-1-methylpyrrolid in-2-yl)methoxy)-7,8-dihydro-5H-pyrano[4,3-d]pyri midin-4-yl)piperazin-2-yl)acetonitrile (Compound 7)

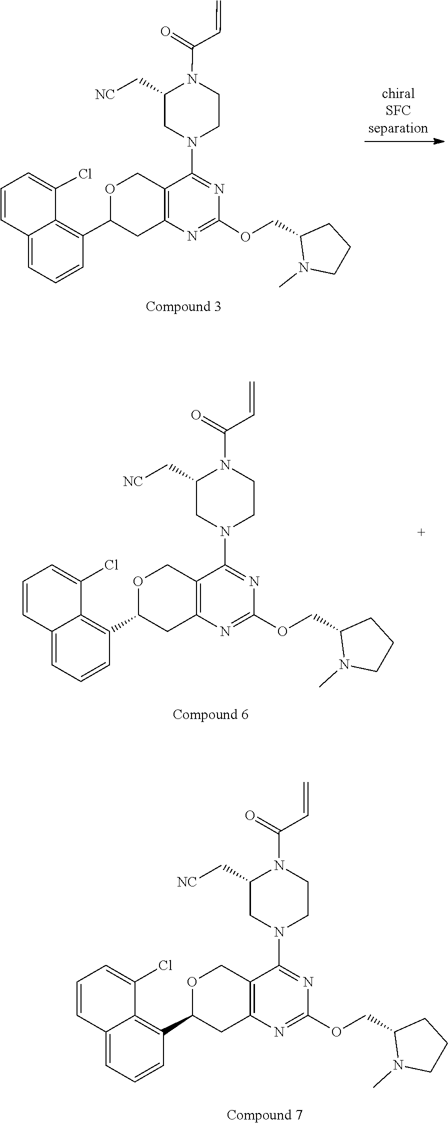 [SMILES: C=CC(=O)N1CCN(c2nc(OC[C@@H]3CCCN3C)nc3c2COC(c2cccc4cccc(Cl)c24)C3)C[C@@H]1CC#N ; C=CC(=O)N1CCN(c2nc(OC[C@@H]3CCCN3C)nc3c2CO[C@@H](c2cccc4cccc(Cl)c24)C3)C[C@@H]1CC#N ; C=CC(=O)N1CCN(c2nc(OC[C@@H]3CCCN3C)nc3c2CO[C@H](c2cccc4cccc(Cl)c24)C3)C[C@@H]1CC#N]

2-((2S)-1-acryloyl-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (96 mg, 0.16 mmol) was isolated by chiral SFC (column: CHIRALPAK OJ-H 250 mm×20 mm, 5 pm; modifier: CO₂ and 40% EtOH (0.2% NH₄OH); flow rate: 40 mL/min) and suitable fractions were pooled and lyophilized to provide 2-((S)-1-acryloyl-4-((R)-7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Compound 6, 34 mg, 35.4%) and 2-((S)-1-acryloyl-4-((S)-7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Compound 7, 35 mg, 36.4%).

Compound 6: LC/MS 586.6 [M+H]⁺; 1H NMR (400 MHz, MeOD) δ 8.02-8.00 (d, J=8.0 Hz, 1H), 7.93-7.91 (d, J=8.0 Hz, 2H), 7.65 (d, J=7.5 Hz, 1H), 7.58 (t, J=7.8 Hz, 1H), 7.42 (t, J=7.8 Hz, 1H), 6.94-6.75 (br, 1H), 6.50-6.47 (dd, J=12.0, 4.0 Hz, 1H), 6.31-6.27 (d, J=12.0 Hz, 1H), 5.85-5.82 (d, J=12.0 Hz, 1H), 5.13-5.09 (d, J=16.0 Hz, 1H), 4.77- 4.55 (br, 1H), 4.47-4.32 (m, 3H), 4.13-4.08 (m, 1H), 3.93-3.90 (d, J=12.0 Hz, 1H), 3.50-3.45 (m, 2H), 3.28-3.22 (m, 3H), 3.12-2.96 (m, 3H), 2.77-2.70 (m, 1H), 2.64-2.55 (m, 4H), 2.20-2.21 (m, 1H), 1.93-1.77 (m, 3H);

Compound 7: LC/MS 586.6 [M+H]⁺; 1H NMR (400 MHz, MeOD) δ 8.02-8.00 (d, J=8.0 Hz, 1H), 7.93-7.91 (d, J=8.0 Hz, 2H), 7.65 (d, J=7.5 Hz, 1H), 7.58 (t, J=7.8 Hz, 1H), 7.42 (t, J=7.8 Hz, 1H), 6.91-6.73 (br, 1H), 6.54-6.50 (dd, J=12.0, 4.0 Hz, 1H), 6.31-6.27 (d, J=12.0 Hz, 1H), 5.85-5.82 (d, J=12.0 Hz, 1H), 5.17-5.13 (d, J=16.0 Hz, 1H), 5.05-4.92(m, 2H), 4.77- 4.74 (d, J=16.0 Hz, 1H), 4.56-4.51 (m, 1H), 4.21-3.99 (m, 3H), 3.88-3.49 (m, 5H), 3.30-3.17 (m, 2H), 3.05 (s, 3H), 2.95- 2.90 (br, 2H), 2.80-2.73 (m, 1H), 2.41-2.34 (m, 1H), 2.21-1.93 (m, 3H).

### Example 4

### Preparation of 2-((2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (compound 53), 2-((S)-4-((R)-7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (compound 54) and 2-((S)-4-((S)-7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (compound 55)

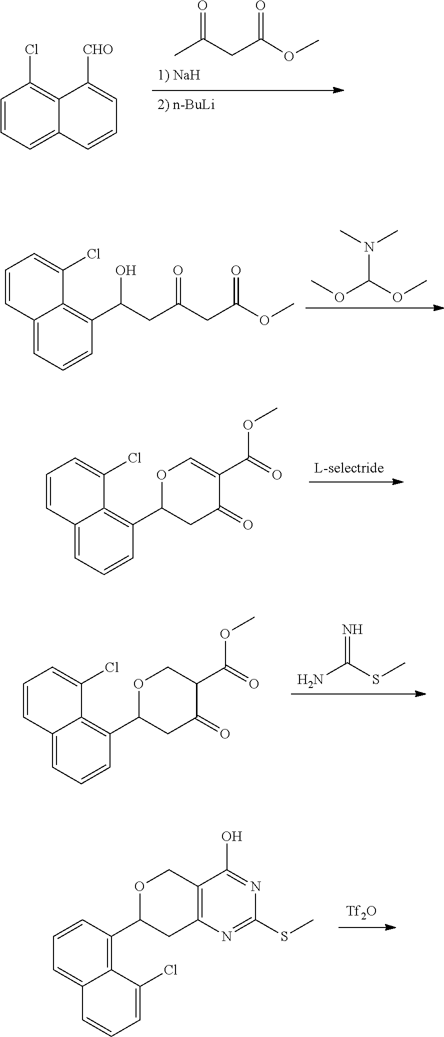

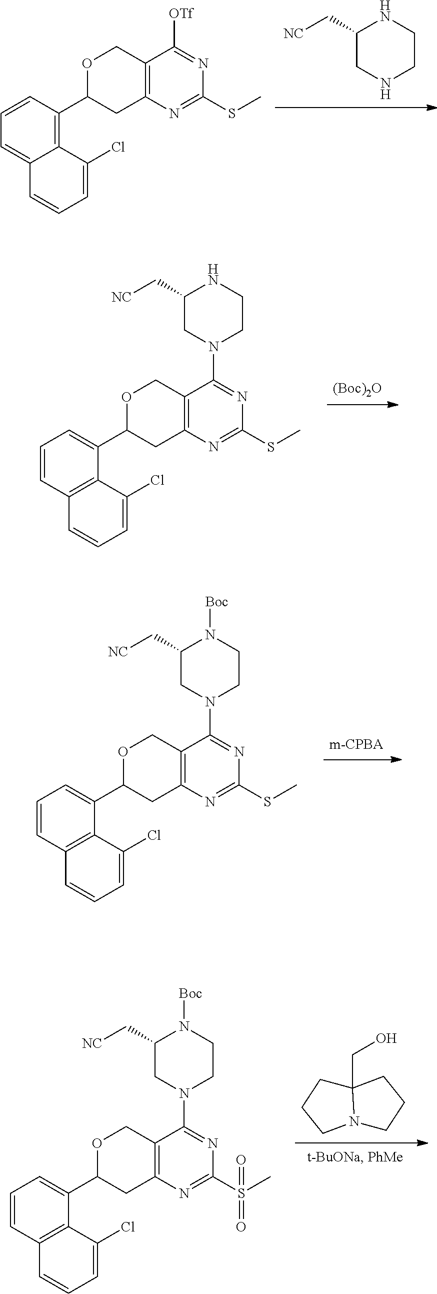

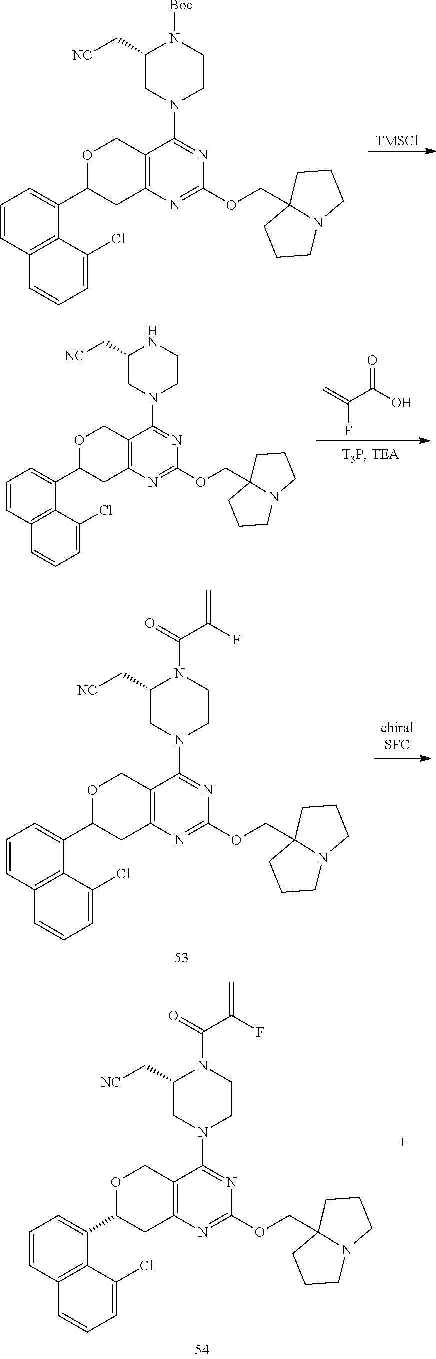

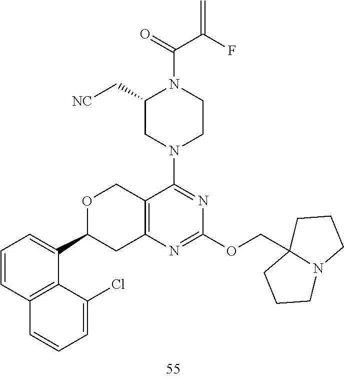 [SMILES: C=C(F)C(=O)N1CCN(c2nc(OCC34CCCN3CCC4)nc3c2CO[C@H](c2cccc4cccc(Cl)c24)C3)C[C@@H]1CC#N]

### Step 1: Preparation of methyl 5-(8-chloronaphthalen-1-yl)-5-hydroxy-3-oxopentanoate

To a solution of methyl 3-oxobutanoate (3.1 g, 26.2 mmol) in THF (80 mL) was added NaH (1.3 g, 31.5 mmol, 60%). After stirring at rt for 30 min, the mixture was cooled to −10° C., n-BuLi (13.1 mL, 31.4 mmol, 2.4 M) was added dropwise. The reaction was stirred for 1 h, and 8-chloro-1-naphthaldehyde (5 g, 26.2 mmol) was added. The resulting solution was stirred overnight. The mixture was poured into aqueous NH₄Cl solution, extracted with EA (200 mL×3). The combined organic layer was dried over Na₂SO₄, filtered and the filtrate was concentrated. The crude product was purified with Combi-Flash (EA/PE=1/2) to give methyl 5-(8-chloronaphthalen-1-yl)-5-hydroxy-3-oxopentanoate (5.3 g, 59.3%) as a yellow solid. LC/MS: 328.8[M+Na]⁺.

### Step 2: Preparation of methyl 2-(8-chloronaphthalen-1-yl)-4-oxo-3,4-dihydro-2H-pyran-5-carboxylate

A mixture of metyl 5-(8-chloronaphthalen-1-yl)-5-hydroxy-3-oxopentanoate (6 g, 22 mmol) and (dimethoxymethyl)dimethylamine (2.9 g, 24.2 mmol) in DCM (200 mL) was stirred at room temperature for 1 h. Then BF₃.Et₂O (3.4 g, 24.2 mmol) was added, the mixture was stirred at rt for 2 hr. DCM (300 mL) was added into the mixture, and the mixture was washed with aqueous NaHCO₃ solution. The organic layer was dried over Na₂SO₄, filtered and the filtrate was concentrated to give methyl 2-(8-chloronaphthalen-1-yl)-4-oxo-3,4-dihydro-2H-pyran-5-carboxylate (6.2 g, 69%) as a yellow oil. The product was used in next step without further purification. LC/MS: 316.8[M+H]⁺.

### Step 3: Preparation of methyl 6-(8-chloronaphthalen-1-yl)-4-oxotetrahydro-2H-pyran-3-carboxylate

To a solution of methyl 2-(8-chloronaphthalen-1-yl)-4-oxo-3,4-dihydro-2H-pyran-5-carboxylate (6.2 g, 22 mmol) in THF (200 mL) was added L-selectride (24.2 mL, 1 M) at −78° C. under N₂ atmosphere. The mixture was stirred at −78° C. for 1 hr, then poured into cooled aqueous NH₄Cl solution and extracted with EA (200 mL×3). The combined organic layers were dried over Na₂SO₄, filtered and the filtrate was concentrated to give 6-(8-chloronaphthalen-1-yl)-4-oxotetrahydro-2H-pyran-3-carboxylate (4.6 g, 68%) as a yellow oil. The product was used in next step without further purification. LC/MS: 318.8[M+H]+.

### Step 4: Preparation of 7-(8-chloronaphthalen-1-yl)-2-(methylthio)-7,8-dihydro-5H-pyrano[4,3-d]pyrimidin-4-ol

To a solution of methyl 6-(8-chloronaphthalen-1-yl)-4-oxooxane-3-carboxylate (4.6 g, 14.4 mmol) and (methylsulfanyl)methanimidamide (1.97 g, 14.4 mmol) in EtOH/H₂O=5:1 (180 mL) was added sodium carbonate (12.2 g, 115.5 mmol) at 25° C. under N₂ atmosphere. The mixture was stirred at 25° C. for 17 hrs. Then the mixture was warmed to 50° C. and stirred for 3 hr. The mixture was filtered and the filtrate was concentrated. The crude residue was diluted with H₂O (50 mL), adjusted to pH=5 with AcOH and extracted with DCM (150 mL×2). The combined organic layers were dried over Na₂SO₄, filtered and the filtrate was concentrated. The crude product was washed with DCM/PE (1:3) and DCM/MTBE (1:15), then filtered. The solid was concentrated to give 7-(8-chloronaphthalen-1-yl)-2-(methylthio)-7,8-dihydro-5H-pyrano[4,3-d]pyrimidin-4-ol (1 g, 17.3%) as a yellow solid. LC/MS: 358.8 [M+H]+.

### Step 5: Preparation of 7-(8-chloronaphthalen-1-yl)-2-(methylthio)-7,8-dihydro-5H-pyrano[4,3-d]pyrimidin-4-yl trifluoromethanesulfonate

To a solution of 7-(8-chloronaphthalen-1-yl)-2-(methylsulfanyl)-5H,7H,8H-pyrano[4,3-d]pyrimidin-4-ol (1 g, 3.0 mmol) in DCM (30 mL) was added DIEA (792.3 mg, 6.1 mmol) and Tf₂O (1.56 g, 5.5 mmol) at 0° C. under N₂ atmosphere. The mixture was stirred at 0° C. for 2 hr. Water (20 mL) was added, and the mixture was extracted with DCM (20 mL×2). The combined organic layers were dried over Na₂SO₄, filtered and the filtrate was concentrated to give 7-(8-chloronaphthalen-1-yl)-2-(methylthio)-7,8-dihydro-5H-pyrano[4,3-d]pyrimidin-4-yl trifluoromethanesulfonate (1.5 g, crude) as a brown solid. The product was used in next step without further purification. LC/MS:490.5 [M+H]⁺.

### Step 6: Preparation of 2-((2S)-4-(7-(8-chloronaphthalen-1-yl)-2-(methylthio)-7,8-dihydro-5H-pyrano[4,3-d]pyrimidin-4-yl)piperazin-2-yl)acetonitrile

To a mixture of 7-(8-chloronaphthalen-1-yl)-2-methoxy-5H,7H,8H-pyrano[4,3-d]pyrimidin-4-yl trifluoromethanesulfonate (1.5 g, crude) and 2-[(2S)-piperazin-2-yl]acetonitrile (514 mg, 4.1 mmol) in MeCN (30 mL) was added DIEA (1.3 g, 9.5 mmol) slowly. The reaction was stirred at 80° C. for 2 hr. The reaction was concentrated to give 2-((2S)-4-(7-(8-chloronaphthalen-1-yl)-2-(methylthio)-7,8-dihydro-5H-pyrano[4,3-d]pyrimidin-4-yl)piperazin-2-yl)acetonitrile (1.8 g, crude) as a brown solid, which was used in next step without further purification. LC/MS:465.7 [M+H]⁺.

### Step 7: Preparation of tert-butyl (2S)-4-(7-(8-chloronaphthalen-1-yl)-2-(methylthio)-7,8-dihydro-5H-pyrano[4,3-d]pyrimidin-4-yl)-2-(cyanomethyl)piperazine-1-carboxylate

To a mixture of 2-((2S)-4-(7-(8-chloronaphthalen-1-yl)-2-(methylthio)-7,8-dihydro-5H-pyrano[4,3-d]pyrimidin-4-yl)piperazin-2-yl)acetonitrile (1.8 g, crude), (Boc)20 (7.4 g, 33.8 mmol) and DIEA (4.4 g, 33.8 mmol) in MeCN (30 mL) was added DMAP (412.5 mg, 3.3 mmol). The reaction was stirred at 25° C. for 60 hrs. The reaction was concentrated and diluted with EA (50 mL). The organic layer was washed with sat. NaHCO₃ solution and brine. The organic layer was dried over Na₂SO₄, filtered and the filtrate was concentrated. The crude product was purified with flash column chromatography (PE/EA=2/1) to provide tert-butyl (2S)-4-(7-(8-chloronaphthalen-1-yl)-2-(methylthio)-7,8-dihydro-5H-pyrano[4,3-d]pyrimidin-4-yl)-2-(cyanomethyl)piperazine-1-carboxylate (390 mg, 16.4% for three steps) as a yellow solid. LC/MS:565.6[M+H]⁺.

### Step 8: Preparation of tert-butyl (2S)-4-(7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate

To a mixture of tert-butyl (2S)-4-[7-(8-chloronaphthalen-1-yl)-2-(methylsulfanyl)-5H,7H,8H-pyrano[4,3-d]pyrimidin-4-yl]-2-(cyanomethyl)piperazine-1-carboxylate (390 mg, 0.69 mmol) in DCM (15 mL) was added 3-chloroperoxybenzoic acid (419.6 mg, 2.1 mmol) slowly. The reaction was stirred at 25° C. for 3 hr. The reaction was diluted with DCM (50 mL), and extracted with sat. Na₂S₂O₃ solution, sat. NaHCO₃ solution and brine. The organic layer was dried over Na₂SO₄, filtered and the filtrate was concentrated. The crude product was purified with flash column chromatography (MeOH/DCM=0-5%) to give tert-butyl (2S)-4-(7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate (340 mg, 74.6%) as a yellow solid. LC/MS:597.5[M+H]⁺.

### Step 9: Preparation of tert-butyl (2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-2-(cyanomethyl)piperazine-1-carboxylate

To a mixture of tert-butyl (2S)-4-(7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate (500 mg, 0.83 mmol) and (tetrahydro-1H-pyrrolizin-7a(5H)-yl)methanol (130 mg, 0.92 mmol) in toluene (40 mL) was added sodium tert-butoxide (120 mg, 1.25 mmol). The reaction mixture was stirred at 0° C. for 2 hr. The reaction was quenched with saturated NH₄Cl solution and extracted with EA (100 mL×3). The organic layer was washed with brine and dried over Na₂SO₄, filtered and the filtrate was concentrated. The residue was purified by silica gel column chromatography (DCM/MeOH=10:1) to give tert-butyl (2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-2-(cyanomethyl)piperazine-1-carboxylate (450 mg, 73.5%) as a yellow solid. LC/MS: 658.7[M+H]⁺.

### Step 10: Preparation of 2-[(2S)-4-[7-(8-chlzoronaphthalen-1-yl)-2-(hexahydropyrrolizin-7a-ylmethoxy)-5H,7H,8H-pyrano[4,3-d]pyrimidin-4-yl]piperazin-2-yl]acetonitrile

To a solution of tert-butyl (2S)-4-(7-(8-chloronaphthalen-1-yl)-2-((tetrahydro-1 H-pyrrolizin-7a(5H)-yl)methoxy)-7,8-dihydro-5H-pyrano[4,3-d]pyrimidin-4-yl)-2-(cyanomethyl)piperazine-1-carboxylate (440 mg, 0.67 mmol) in DCM (15 mL) was added TMSCI (218 mg, 2.01 mmol). The resulting solution was stirred at r.t. for 1 hr. After evaporation under vacuum, 2-[(2S)-4-[7-(8-chlzoronaphthalen-1-yl)-2-(hexahydropyrrolizin-7a-ylmethoxy)-5H,7H,8H-pyrano[4,3-d]pyrimidin-4-yl]piperazin-2-yl]acetonitrile (350 mg, 84.5%) was obtained as a yellow solid, which was used in next step without further purification. LC/MS: 558.7[M+H]⁺.

### Step 11: Preparation of 2-[(2S)-4-[7-(8-chloronaphthalen-1-yl)-2-(hexahydropyrrolizin-7a-ylmethoxy)-5H,7H,8H-pyrano[4,3-d]pyrimidin-4-yl]-1-(2-fluoroprop-2-enoyl)piperazin-2-yl]acetonitrile

A mixture of 2-[(2S)-4-[7-(8-chloronaphthalen-1-yl)-2-(hexahydropyrrolizin-7a-ylmethoxy)-5H,7H,8H-pyrano[4,3-d]pyrimidin-4-yl]piperazin-2-yl]acetonitrile (350 mg, 0.63 mmol), 2-fluoroprop-2-enoic acid (113 mg, 1.25 mmol), triethylamine (316 mg, 3.13 mmol) and T₃P (50% in EA, 1.59 g, 2.50 mmol) in DCM (40 mL) was stirred at r.t. for 15 hr. The reaction mixture was concentrated under vacuum. The crude residue was purified by Prep-HPLC using a gradient of 0.1% TFA/ACN from 75% to 45%, and pure fractions were pooled and lyophilized to give 2-[(2S)-4-[7-(8-chloronaphthalen-1-yl)-2-(hexahydropyrrolizin-7a-ylmethoxy)-5H,7H,8H-pyrano[4,3-d]pyrimidin-4-yl]-1-(2-fluoroprop-2-enoyl)piperazin-2-yl]acetonitrile (Compound 53, 150 mg, 36.1%) as a white solid. LC/MS: 631.2[M+H]⁺.

¹H NMR (400 MHz, CD₃OD) δ 8.05 (d, J=7.5 Hz, 1H), 8.00-7.91 (m, 2H), 7.73-7.67 (m, 1H), 7.62 (t, J=7.8 Hz, 1H), 7.50-7.44 (m, 1H), 6.58-6.49 (m, 1H), 5.47-5.29 (m, 2H), 5.25-5.17 (m, 1H), 5.07-4.96 (m, 1H), 4.87-4.79 (m, 1H), 4.68-4.60 (m, 1.5H), 4.43-4.36 (m, 0.5H), 4.27-3.97 (m, 2H), 3.81-3.39 (m, 6H), 3.31-3.24 (m, 2H), 3.21-3.09 (m, 1H), 3.06-2.77 (m, 3H), 2.41-2.05 (m, 8H).

The racemate mixture of 2-[(2S)-4-[7-(8-chloronaphthalen-1-yl)-2-(hexahydropyrrolizin-7a-ylmethoxy)-5H,7H,8H-pyrano[4,3-d]pyrimidin-4-yl]-1-(2-fluoroprop-2-enoyl)piperazin-2-yl]acetonitrile (120 mg, 0.19 mmol) was separated by chiral SFC (Column: CHIRALPAK OJ-H 250 mm×20 mm, 5 μm; Modifier: CO₂ and 40% MeOH (0.2% NH₄OH); Total Flow: 40 g/min). Pure fractions of each peak were pooled and lyophilized to give the first eluting peak 2-[(2S)-4-[(7R)-7-(8-chloronaphthalen-1-yl)-2-(hexahydropyrrolizin-7a-ylmethoxy)-5H,7H,8H-pyrano[4,3-d]pyrimidin-4-yl]-1-(2-fluoroprop-2-enoyl)piperazin-2-yl]acetonitrile (Compound 54, 27.9 mg, 23%) and the second eluting peak 2-[(2S)-4-[(7S)-7-(8-chloronaphthalen-1-yl)-2-(hexahydropyrrolizin-7a-ylmethoxy)-5H,7H,8H-pyrano[4,3-d]pyrimidin-4-yl]-1-(2-fluoroprop-2-enoyl)piperazin-2-yl]acetonitrile (Compound 55, 38 mg, 31%).

2-[(2S)-4-[(7R)-7-(8-chloronaphthalen-1-yl)-2-(hexahydropyrrolizin-7a-ylmethoxy)-5H,7H,8H-pyrano[4,3-d]pyrimidin-4-yl]-1-(2-fluoroprop-2-enoyl)piperazin-2-yl]acetonitrile (Compound 54)

¹H NMR (400 MHz, CD₃OD) δ 8.04 (d, J=7.5 Hz, 1H), 7.95 (d, J=8.2 Hz, 2H), 7.68 (dd, J=7.5, 1.2 Hz, 1H), 7.61 (t, J=7.8 Hz, 1H), 7.48-7.43 (m, 1H), 6.51 (dd, J=10.9, 3.1 Hz, 1H), 5.46-5.27 (m, 3H), 5.18-5.13 (m, 1H), 4.97-4.93 (m, 1H), 4.57-4.51 (m,1H), 4.47-4.36 (m, 2H), 4.14-4.01 (m, 1H), 3.98-3.91 (m, 1H), 3.59-3.39 (m, 5H), 3.23-3.02 (m, 5H), 2.80-2.73 (m, 1H), 2.25-1.92 (m, 8H).

### 2-[(2S)-4-[(7S)-7-(8-chloronaphthalen-1-yl)-2-(hexahydropyrrolizin-7a-ylmethoxy)-5H,7H,8H-pyrano[4,3-d]pyrimidin-4-yl]-1-(2-fluoroprop-2-enoyl)piperazin-2-yl]acetonitrile (Compound 55)

¹H NMR (400 MHz, CD₃OD) δ 8.05 (d, J=7.1 Hz, 1H), 7.98-7.92 (m, 2H), 7.68 (dd, J=7.5, 1.2 Hz, 1H), 7.61 (t, J=7.8 Hz, 1H), 7.48-7.43 (m, 1H), 6.54 (dd, J=10.8, 3.1 Hz, 1H), 5.47-5.25 (m, 3H), 5.19-5.13 (m, 1H), 5.02-4.97 (m, 1H), 4.54 (s, 2H), 4.31-4.12 (m, 2H), 4.07-4.01 (m, 1H), 3.75-3.50 (m, 5H), 3.29-3.14 (m, 3H), 3.05-2.92 (m, 2H), 2.84-2.75 (m, 1H), 2.35-2.06 (m, 8H).

Example 5: Preparation of 2-((2S)-1-(2-fluoroacryloyl)-4-(7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (compound 56), 2-((S)-1-(2-fluoroacryloyl)-4-((R)-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (compound 57) and 2-((S)-1-(2-fluoroacryloyl)-4-((S)-7-(8-methylnaphthalen-1-yl)-2-((tetrahydro-1H-pyrrolizin-7a(5H)-yl)methoxy)-7,8-dihydro-5H-pyrano[4,3-d]pyri midi n-4-yl)piperazin-2-yl)acetonitrile (compound 58)

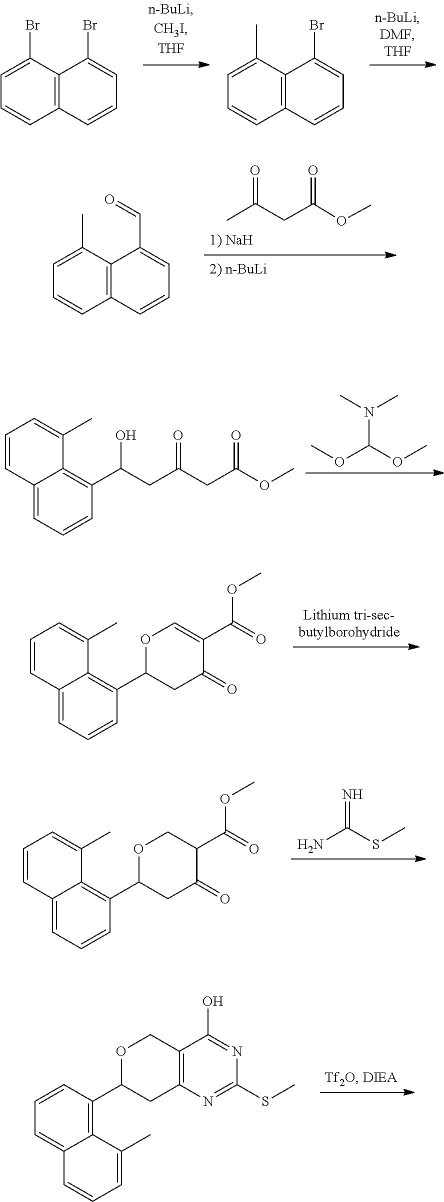

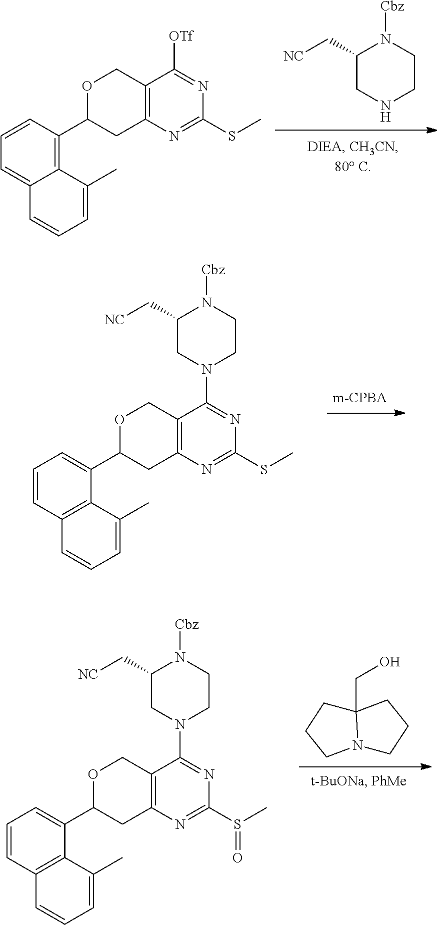 [SMILES: CSc1nc2c(c(OS(=O)(=O)C(F)(F)F)n1)COC(c1cccc3cccc(C)c13)C2 ; N#CC[C@H]1CNCCN1C(=O)OCc1ccccc1 ; CSc1nc2c(c(N3CCN(C(=O)OCc4ccccc4)[C@@H](CC#N)C3)n1)COC(c1cccc3cccc(C)c13)C2 ; Cc1cccc2cccc(C3Cc4nc(S(C)=O)nc(N5CCN(C(=O)OCc6ccccc6)[C@@H](CC#N)C5)c4CO3)c12 ; OCC12CCCN1CCC2]

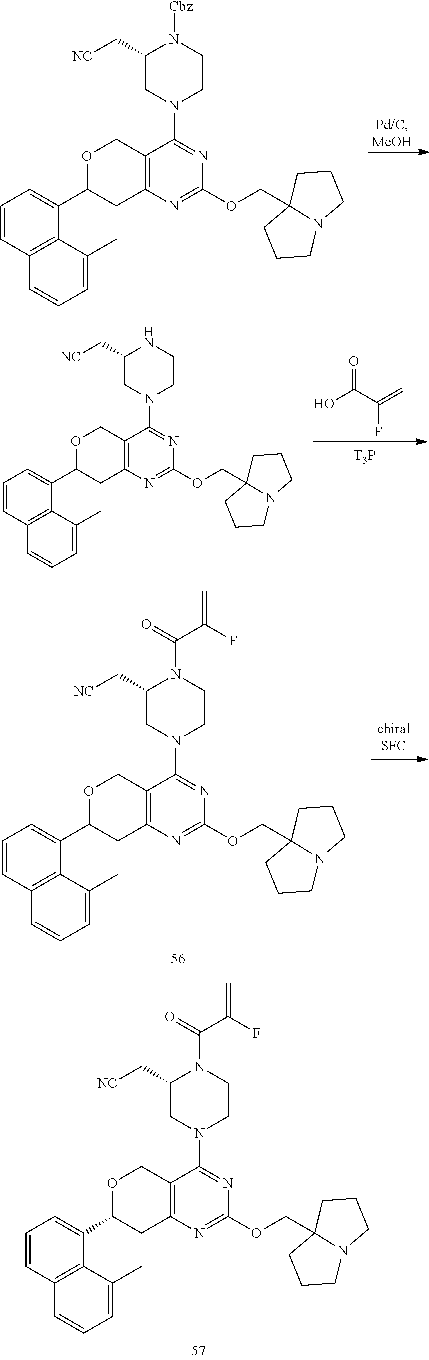

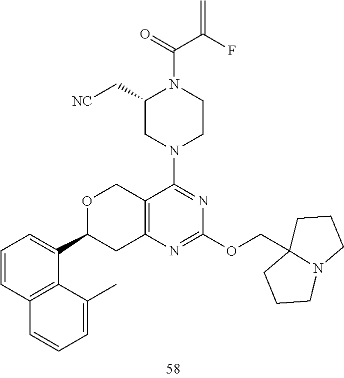 [SMILES: C=C(F)C(=O)N1CCN(c2nc(OCC34CCCN3CCC4)nc3c2CO[C@H](c2cccc4cccc(C)c24)C3)C[C@@H]1CC#N]

### Step 1: Preparation of 1-bromo-8-methylnaphthalene

To a solution of 1,8-dibromonaphthalene (50 g, 174.8 mmol) in THF (500 mL) was added n-BuLi (2.4M, 75 mL) at −70° C. After stirring at −70° C. for 1 hr, CH₃I (27.2g, 192.2 mmol) was added slowly. The reaction mixture was stirred at 25° C. for 1 hr. The reaction was quenched with saturated aqueous NH₄Cl (500 mL) and extracted with EtOAc (1000 mL×2). The combined organic layer was washed with brine, dried over Na₂SO₄ and filtered. The filtrate was concentrated under vacuum and the residue was purified by column chromatography (eluted by EA/PE=0-1%) to give 1-bromo-8-methylnaphthalene (37 g, 97%) as a white solid.

### Step 2: Preparation of 8-methyl-1-naphthaldehyde

To a solution of 1-bromo-8-methylnaphthalene (37 g, 167.4 mmol) in THF (300 mL) was added n-BuLi (2.4M, 77 mL) at −70° C. After stirring at −70° C. for 1 hr. DMF (14.7 g, 200.8 mmol) was added slowly. The mixture was stirred at −70° C. for 1 hr. The reaction was quenched with saturated aqueous NH₄Cl (200 mL) and extracted with EtOAc (500 mL×2). The combined organic phase was washed with brine, dried over Na₂SO₄ and filtered. The filtrate was concentrated under vacuum and the residue was purified by column chromatography (EA/PE=0-10%) to give 8-methylnaphthalene-1-carbaldehyde carbaldehyde (22 g, 78%) as a yellow solid.

### Step 3: Preparation of methyl 5-hydroxy-5-(8-methylnaphthalen-1-yl)-3-oxopentanoate

To a solution of methyl 3-oxobutanoate (15 g, 129.3 mmol) in THF (300 mL) was added NaH (6.2 g, 60% dispersion, 155 mmol). After stirring at 0° C. for 1 hr., n-BuLi (65 mL, 155 mmol) was added. The reaction was stirred for additional 45 min. 8-Methylnaphthalene-1-carbaldehyde (22 g, 129.3 mmol) was added and the reaction was stirred at r.t. overnight. The mixture was diluted with EtOAc (200 mL), poured into ice NH₄Cl and extracted with EtOAc (200 mL×2). The combined organic layers was dried over Na₂SO₄ and filtered. The filtrate was concentrated under vacuum and the residue was purified by column chromatography (EA/PE=From 5:1 to 2:1) to give methyl 5-hydroxy-5-(8-methylnaphthalen-1-yl)-3-oxopentanoate (31 g, 83%) as a yellow solid.

### Step 4: Preparation of methyl 2-(8-methylnaphthalen-1-yl)-4-oxo-3,4-dihydro-2H-pyran-5-carboxylate

A mixture of methyl 5-hydroxy-5-(8-methylnaphthalen-1-yl)-3-oxopentanoate (13 g, 45.4 mmol) and (dimethoxymethyl)dimethylamine (5.95 g, 49.6 mmol) in DCM (300 mL) was stirred at room temperature for 1 hr. BF₃.Et₂O (6.44 g, 45.4 mmol) was added and the mixture was stirred at r.t. for 2 hr. The reaction was diluted with DCM (300 mL) and extracted with aqueous NaHCO₃ solution. The organic layer was dried over Na₂SO₄, filtered and the filtrate was concentrated to give methyl 6-(8-methylnaphthalen-1-yl)-4-oxo-5,6-dihydropyran-3-carboxylate (13 g) as a yellow oil which was used in the next step without further purification. LC/MS: 296.8[M+H]⁺.

### Step 5: Preparation of methyl 6-(8-methylnaphthalen-1-yl)-4-oxotetrahydro-2H-pyran-3-carboxylate

To a solution of methyl 2-(8-chloronaphthalen-1-yl)-4-oxo-3,4-dihydro-2H-pyran-5-carboxylate (13 g, 43.9 mmol) in THF (400 mL) was added L-selectride (48 mL, 1 M) at −78° C. under N₂ atmosphere. After stirring at −78° C. for 1 hr., the mixture was poured into cooled aqueous NH₄Cl solution and extracted with EtOAc (200 mL×3). The combined organic layers was dried over Na₂SO₄ and filtered. The filtrate was concentrated in vacuo to give 6-(8-chloronaphthalen-1-yl)-4-oxotetrahydro-2H-pyran-3-carboxylate (10 g) as a yellow oil which was used in the next step without further purification.

### Step 6: Preparation of 7-(8-methylnaphthalen-1-yl)-2-(methylthio)-7,8-dihydro-5H-pyrano[4,3-d]pyrimidin-4-ol

To a solution of methyl 6-(8-chloronaphthalen-1-yl)-4-oxooxane-3-carboxylate (10 g, 33.5 mmol) and (methylsulfanyl)methanimidamide (3.02 g, 33.5 mmol) in EtOH/H₂O=5:1 (120 mL) was added sodium carbonate (35 g, 0.33 mol) at 25° C. under N₂ atmosphere. The mixture was stirred at 25° C. for 17 hr. The reaction mixture was warmed to 50° C. and stirred for 3 hr. The mixture was filtered and the filtrate was concentrated in vacuo. The residue was dissolved with water (150 mL), adjusted to pH=5 with AcOH and extracted with DCM (100 mL×2). The combined organic layers was dried over Na₂SO₄, filtered and the filtrate was concentrated in vacuo. The residue was triturated with PE/DCM (10:1) to give 7-(8-methylnaphthalen-1-yl)-2-(methylsulfanyl)-5H,7H,8H-pyrano[4,3-d]pyrimidin-4-ol (3 g , 27%) as a yellow solid. LC/MS: 338.8[M+H]⁺.

### Step 7: Preparation of 7-(8-methylnaphthalen-1-yl)-2-(methylthio)-7,8-dihydro-5H-pyrano[4,3-d]pyrimidin-4-yl trifluoromethanesulfonate

To a solution of 7-(8-methylnaphthalen-1-yl)-2-(methylsulfanyl)-5H,7H,8H-pyrano[4,3-d]pyrimidin-4-ol (3 g, 8.8 mmol) in DCM (100 mL) was added DIEA (3.43 g, 26.59 mmol) and Tf₂O (5 g, 17.72 mmol) at 0° C. under N₂ atmosphere. The reaction mixture was stirred at 0° C. for 2 hr. Water (20 mL) was added to the mixture and extracted with DCM (200 mL×2). The combined organic layers was dried over Na₂SO₄ and filtered. The filtrate was concentrated to give 7-(8-methylnaphthalen-1-yl)-2-(methylsulfanyl)-5H,7H,8H-pyrano[4,3-d]pyrimidin-4-yl trifluoromethanesulfonate (3 g) as a brown oil which was used in the next step without further purification. LC/MS: 470.7[M+H]⁺.

### Step 8: Preparation of benzyl (2S)-2-(cyanomethyl)-4-(7-(8-methylnaphthalen-1-yl)-2-(methylthio)-7,8-dihydro-5H-pyrano[4,3-d]pyrimidin-4-yl)piperazine-1-carboxylate

To a solution of 7-(8-methylnaphthalen-1-yl)-2-(methylsulfanyl)-5H,7H,8H-pyrano[4,3-d]pyrimidin-4-yl trifluoromethanesulfonate (3 g) and benzyl (5)-2-(cyanomethyl)piperazine-1-carboxylate (1.65 g, 6.38 mmol) in CH₃CN (50 mL) was added ethyldiisopropylamine (1.6 g, 12.75 mmol). The mixture was stirred at 80° C. for 1 hr. The mixture was diluted with EtOAc (50 mL), poured into ice water and extracted with EtOAc (100 mL×2). The combined organic layer was dried over Na₂SO₄ and concentrated under vacuum. The crude product was purified by column chromatography (EA/PE=0-30%) to give benzyl (2S)-2-(cyanomethyl)-4-(7-(8-methylnaphthalen-1-yl)-2-(methylthio)-7,8-dihydro-5H-pyrano[4,3-d]pyrimidin-4-yl)piperazine-1-carboxylate (3 g) as a yellow oil which was used in the next step without further purification. LC/MS: 580.3[M+H]⁺.

### Step 9: Preparation of benzyl (2S)-2-(cyanomethyl)-4-(7-(8-methylnaphthalen-1-yl)-2-(methylsulfonyl)-7,8-dihydro-5H-pyrano[4,3-d]pyrimidin-4-yl)piperazine-1-carboxylate

To a mixture of benzyl (2S)-2-(cyanomethyl)-4-(7-(8-methylnaphthalen-1-yl)-2-(methylthio)-7,8-dihydro-5H-pyrano[4,3-d]pyrimidin-4-yl)piperazine-1-carboxylate (3 g, 5.16 mmol) in DCM (55 mL) was added 3-Chloroperoxybenzoic acid (2.67 g, 15 mmol). After stirring at 25° C. for 3 hr., the reaction was diluted with DCM (50 mL) and extracted with sat. Na₂S₂O₃ solution, sat. NaHCO₃ solution and brine sequentially. The organic layer was dried over Na₂SO₄, filtered and the filtrate was concentrated under vacuum. The crude product was purified by column chromatography (MeOH/DCM=0-5%) to give benzyl (2S)-2-(cyanomethyl)-4-(7-(8-methylnaphthalen-1-yl)-2-(methylsulfonyl)-7,8-dihydro-5H-pyrano[4,3-d]pyrimidin-4-yl)piperazine-1-carboxylate (1.2 g, impure) as a yellow solid. LC/MS: 611.6[M+H]⁺.

### Step 10: Preparation of benzyl (2S)-2-(cyanomethyl)-4-(7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazine-1-carboxylate

To a solution of hexahydropyrrolizin-7a-ylmethanol (118 mg, 0.83 mmol) and benzyl (2S)-2-(cyanomethyl)-4-(7-(8-methylnaphthalen-1-yl)-2-(methylsulfonyl)-7,8-dihydro-5H-pyrano[4,3-d]pyrimidin-4-yl)piperazine-1-carboxylate in THF (50 mL) was added t-BuONa (242 mg, 2.51 mmol). The mixture was stirred at 20° C. for 1 hr. and then diluted with EtOAc (20 mL). The mixture was poured into ice water and extracted with EtOAc (20 mL×2). The combined organic layers were dried over Na₂SO₄ and concentrated under vacuum. The crude product was purified by column chromatography (DCM/MeOH=0-20%) to give benzyl (2S)-2-(cyanomethyl)-4-(7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazine-1-carboxylate (350 mg , 63%) as a yellow oil.

### Step 11: Preparation of 2-((2S)-4-(7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile

To a mixture of benzyl (2S)-2-(cyanomethyl)-4-(7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazine-1-carboxylate (350 mg, 0.52 mmol) in CH₃OH (15 mL) was added Pd/C (200 mg). The reaction was stirred at 20° C. for 4 hr under H₂ atmosphere. The reaction mixture was filtered and the filtrate was concentrated to give 2-[(2S)-4-[2-(hexahydropyrrolizin-7a-ylmethoxy)-7-(8-methylnaphthalen-1-yl)-5H,7H,8H-pyrano[4,3-d]pyrimidin-4-yl]piperazin-2-yl]acetonitrile (140 mg) as a yellow solid which was used in the next step without further purification. LC/MS: 539.4 [M+H]⁺.

### Step 12: Preparation of 2-((2S)-1-(2-fluoroacryloyl)-4-(7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (compound 56)

To a mixture of 2-[(2S)-4-[2-(hexahydropyrrolizin-7a-ylmethoxy)-7-(8-methylnaphthalen-1-yl)-5H,7H,8H-pyrano[4,3-d]pyrimidin-4-yl]piperazin-2-yl]acetonitrile (140 mg, 0.26 mmol) and 2-fluoroprop-2-enoic acid (47 mg, 0.51 mmol) in DCM (100 mL) was added Et₃N (131 mg, 1.29 mmol) and T₃P (50% in EtOAc, 826 mg, 1.3 mmol). After stirring at 20° C. for 16 hr., the reaction mixture was concentrated under vacuum and purified by Prep-HPLC using a gradient of 0.1% FA/ACN from 70% to 40% to give 2-((2S)-1-(2-fluoroacryloyl)-4-(7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (Compound 56, 120 mg, 76%) as a white solid. LC/MS: 610.8[M+H]⁺.

1H NMR (400 MHz, CD₃OD) δ 7.95-7.77 (m, 3H), 7.51 (t, J=7.7 Hz, 1H), 7.44-7.35 (m, 2H), 6.16-6.04 (m, 1H), 5.49-5.27 (m, 2H), 5.16 (d, J=13.5 Hz, 1H), 4.89-4.74 (m, 2H), 4.73-4.55 (m, 2H), 4.54-3.94 (m, 3H), 3.64 (m, 5H), 3.32-3.12 (m, 4H), 3.08-2.92 (m, 5H), 2.42-2.00 (m, 8H).

The racemic mixture of 2-[(2S)-1-(2-fluoroprop-2-enoyl)-4-[2-(hexahydropyrrolizin-7a-ylmethoxy)-7-(8-methylnaphthalen-1-yl)-5H,7H,8H-pyrano[4,3-d]pyrimidin-4-yl]piperazin-2-yl]acetonitrile (110 mg, 0.18 mmol) was separated by chiral SFC (Column: CHIRALPAK OJ-H 250 mm×20 mm, 5 μm; modifier: CO₂ and 40% EtOH (0.2% NH₄OH); Total Flow: 40 g/min). Pure fractions of each peak were pooled and lyophilized to give 2-((S)-1-(2-fluoroacryloyl)-4-((S)-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (compound 58, first peak, 35 mg, 32%) and 2-((S)-1-(2-fluoroacryloyl)-4-((R)-7-(8-methylnaphthalen-1-yl)-2-((tetrahydro-1 H-pyrrolizin-7a(5H)-yl)methoxy)-7,8-dihydro-5H-pyrano[4,3-d]pyrimidin-4-yl)piperazin-2-yl)acetonitrile (compound 57, second peak, 48 mg, 44%). LC/MS: 610.8[M+H]⁺.

2-((S)-1-(2-fluoroacryloyl)-4-((S)-7-(8-methylnaphthalen-1-yl)-2-((tetrahydro-1 H-pyrrolizin-7a(5H)-yl)methoxy)-7,8-dihydro-5H-pyrano[4,3-d]pyrimidin-4-yl)piperazin-2-yl)acetonitrile (compound 58)

¹H NMR (400 MHz, CD₃OD) δ 7.93-7.74 (m, 3H), 7.48 (t, J=7.7 Hz, 1H), 7.44-7.27 (m, 2H), 6.07 (dd, J=9.9, 3.7 Hz, 1H), 5.48-5.18 (m, 2H), 5.07 (d, J=14.0 Hz, 1H), 4.78 (d, J=13.9 Hz, 1H), 4.36 (d, J=14.1 Hz, 1H), 4.26-4.18 (m, 2H), 3.91 (d, J=11.0 Hz, 1H), 3.32-3.04 (m, 10H), 3.01-2.79 (m, 6H), 2.15-1.76 (m, 8H).

2-((S)-1-(2-fluoroacryloyl)-4-((R)-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (compound 57)

1H NMR (400 MHz, CD₃OD) δ 7.87 (t, J=7.2 Hz, 2H), 7.83-7.73 (m, 1H), 7.50 (t, J=7.7 Hz, 1H), 7.43-7.31 (m, 2H), 6.09 (d, J=6.9 Hz, 1H), 5.45-5.27 (m, 2H), 5.12 (d, J=14.1 Hz, 1H), 4.88 (d, J=4.7 Hz, 1H), 4.54 (s, 2H), 4.23 (d, J=14.8 Hz, 1H), 4.03 (d, J=12.7 Hz, 1H), 3.73-3.47 (m, 4H), 3.32-3.09 (m, 7H), 3.06-2.87 (m, 5H), 2.39-2.04 (m, 8H).

### Example 6

### Preparation of 2-((2S)-4-(7-(8-chloronaphthalen-1-yl)-5-methyl-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (compound 59)

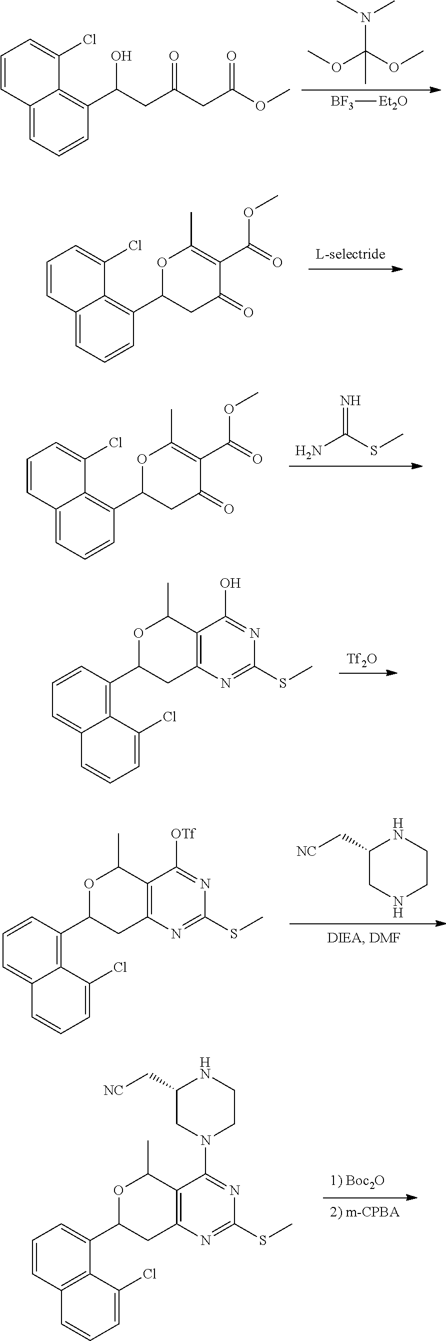

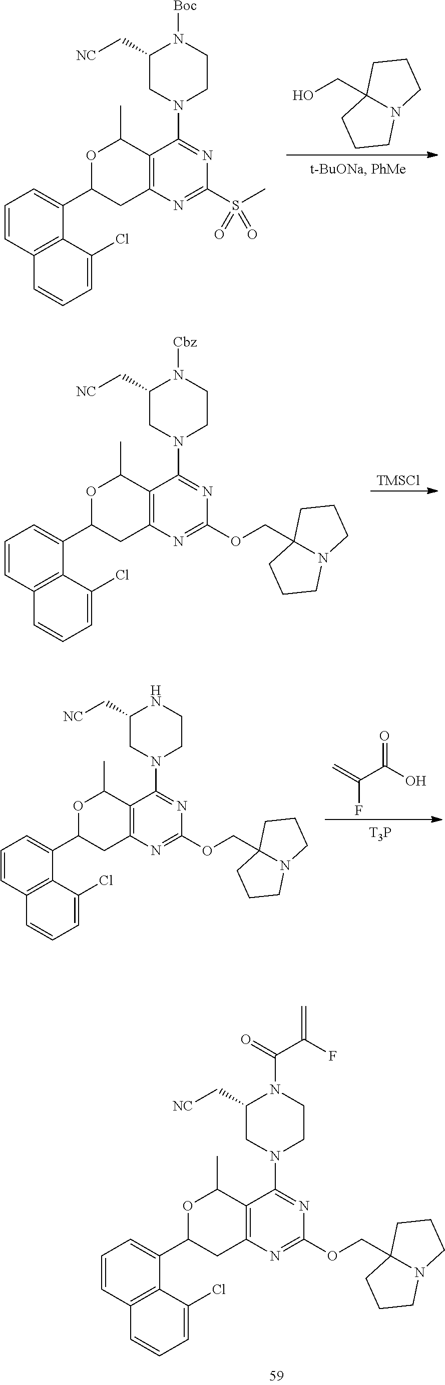

### Step 1: Preparation of methyl 2-(8-chloronaphthalen-1-yl)-6-methyl-4-oxo-3,4-dihydro-2H-pyran-5-carboxylate

To a solution of methyl 5-(8-chloronaphthalen-1-yl)-5-hydroxy-3-oxopentanoate (8.5 g, 27.7 mmol) and 1,1-dimethoxy-N,N-dimethylethan-1-amine (4.06 g, 30.5 mmol) in DCM (50 mL) stirred at r.t. for 45 mins was added BF₃.Et₂O (3.93 g, 27.7 mmol). The reaction was stirred at r.t. for additional 2 hr. EtOAc (50 mL) was added to the reaction mixture. The reaction mixture was extracted with sat. NaHCO₃ solution, dried over Na₂SO₄ and filtered. The filtrate was concentrated and purified with column chromatography (EA/PE=0-15%) to give the titled product (6.5 g, 71%) as a yellow oil. LC/MS: 331.0 [M+H]⁺.

### Step 2: Preparation of methyl 6-(8-chloronaphthalen-1-yl)-2-methyl-4-oxooxane-3-carboxylate

To a solution of methyl 6-(naphthalen-1-yl)-4-oxo-5,6-dihydropyran-3-carboxylate (6.5 g, 19.7 mmol) in THF (100 mL) was added L-selectride (1 M, 20 mL, 20 mmol) at −78° C. The reaction was stirred at −78° C. for 1 hr. EtOAc (100 mL) was added and the mixture was poured into ice NH₄Cl solution. The organic layer was concentrated to dryness and the residue was purified by column chromatography (EA/PE=0-35%) to give the titled product (5.0 g, 77%) as a pale-yellow oil. LC/MS: 355.0 [M+H]⁺.

### Step 3: Preparation of 7-(8-chloronaphthalen-1-yl)-5-methyl-2-(methylsulfanyl)-5H,7H,8H-pyrano[4,3-d]pyrimidin-4-ol

To a solution of methyl 6-(naphthalen-1-yl)-4-oxooxane-3-carboxylate (5 g, 15.1 mmol) and (methylsulfanyl)methanimidamide (2.83 g, 15.1 mmol) in EtOH/H20=5:1 (120 mL) was added Na₂CO₃ (16 g, 151 mmol) at 25° C. under N₂ atmosphere. The resulting reaction mixture was stirred at 25° C. for 17 hr. The mixture was warmed to 50° C. and stirred for 3 hr. The mixture was filtered and the filtrate was concentrated under vacuum. The crude mixture was diluted with H₂O (50 mL), adjusted to pH=5 with AcOH, extracted with DCM (100 mL×2). The combined organic layer was dried over Na₂SO₄, filtered and the filtrate was concentrated. The crude mixture was triturated with DCM/PE (1:3) and DCM/MTBE (1:15). The solid was filtered and dried to give 7-(8-chloronaphthalen-1-yl)-2-(methylsulfanyl)-5H,7H,8H-pyrano[4,3-d]pyrimidin-4-ol (1.3 g, 23%) as a white solid. LC/MS: 373.0 [M+H]⁺.

### Step 4: Preparation of 7-(8-chloronaphthalen-1-yl)-5-methyl-2-(methylsulfanyl)-5H,7H,8H-pyrano[4,3-d]pyrimidin-4-yl trifluoromethanesulfonate

To a solution of 7-(8-chloronaphthalen-1-yl)-2-(methylsulfanyl)-5H,7H,8H-pyrano[4,3-d]pyrimidin-4-ol (3.6 g, 9.7 mmol) in DCM (50 mL) was added DIEA (2.51 g, 19.4 mmol) and Tf₂O (4.93 g, 17.5 mmol) at 0° C. under N₂ atmosphere. The mixture was stirred at 0° C. for 2 hr. Water (20 mL) was added to the reaction mixture and extracted with DCM (50 mL×2). The organic layer was concentrated and purified by column chromatography (EA/PE=0-15%) to give 7-(8-chloronaphthalen-1-yl)-5-methyl-2-(methylsulfanyl)-5H,7H,8H-pyrano[4,3-d]pyrimidin-4-yl trifluoromethanesulfonate (3.9 g, 80%) as a yellow oil. LC/MS: 504.9[M+H]⁺.

Step 5: Preparation of 2-[(2S)-4-[7-(8-chloronaphthalen-1-yl)-5-methyl-2-(methylsulfanyl)-5H,7H,8H-pyrano[4,3-d]pyrimidin-4-yl]piperazin-2-yl]acetonitrile To a solution of 7-(8-chloronaphthalen-1-yl)-5-methyl-2-(methylsulfanyl)-5H,7H,8H-pyrano[4,3-d]pyrimidin-4-yl trifluoromethanesulfonate (3.9 g, 7.7 mmol) and 2-[(2S)-piperazin-2-yl]acetonitrile (0.97 g, 7.7 mmol) in CH₃CN (50 mL) was added DIEA (2.0 g, 15.4 mmol). The mixture was stirred at 100° C. for 1 hr. and then concentrated under vacuum. The crude product was used for the next step without further purification. LC/MS: 479.7[M+H]⁺.

### Step 6: Preparation of tert-butyl (2S)-4-[7-(8-chloronaphthalen-1-yl)-5-methyl-2-(methylsulfanyl)-5H,7H,8H-pyrano[4,3-d]pyrimidin-4-yl]-2-(cyanomethyl)piperazine-1-carboxylate

To a solution of 2-[(2S)-4-[7-(8-chloronaphthalen-1-yl)-2-{[(2S)-1-methylpyrrolidin-2-yl]methoxy}-5H,7H,8H-pyrano[4,3-d]pyrimidin-4-yl]piperazin-2-yl]acetonitrile (3.9 g, 8.1 mmol), (Boc)20 (8.77 g, 40.6 mmol) and DIEA (5.24 g, 40.6 mmol) in DCM (40 mL) was added DMAP (988 mg, 8.1 mmol) portion wise. The reaction was stirred at 25° C. for 24 hr. The reaction mixture was concentrated and diluted with EtOAc (100 mL). The organic layer was washed with sat. NaHCO₃ solution and brine, dried over Na₂SO₄, filtered and the filtrate was concentrated and purified with column chromatography (EA/PE=0-20%) to give tert-butyl (2S)-4-[7-(8-chloronaphthalen-1-yl)-5-methyl-2-(methylsulfanyl)-5H,7H,8H-pyrano[4,3-d]pyrimidin-4-yl]-2-(cyanomethyl)piperazine-1-carboxylate (690 mg, 15% for two steps) as a yellow solid. LC/MS: 504.9[M+H]⁺.

### Step 7: Preparation of tert-butyl (2S)-4-(7-(8-chloronaphthalen-1-yl)-5-methyl-2-((tetrahydro-1H-pyrrolizin-7a(5H)-yl)methoxy)-7,8-dihydro-5H-pyrano[4,3-d]pyrimidin-4-yl)-2-(cyanomethyl) piperazine-1-carboxylate

To a mixture of tert-butyl (2S)-4-[7-(8-chloronaphthalen-1-yl)-2-methanesulfonyl-5-methyl-5H,7H,8H-pyrano[4,3-d]pyrimidin-4-yl]-2-(cyanomethyl)piperazine-1-carboxylate (400 mg, 0.65 mmol) and hexahydropyrrolizin-7a-ylmethanol (184 mg, 1.30 mmol) in toluene (7 mL) was added sodium tert-butoxide (125 mg, 1.3 mmol). The reaction was stirred at 20° C. for 4 hr. The reaction mixture was diluted with EA (10 mL). The organic layer was washed with brine, dried over Na₂SO₄ and filtered. The filtrate was concentrated and purified by Combi-Flash (eluted by DCM/MeOH=10/1) to give the product (280 mg, 64%) as a yellow solid. LC/MS: 672.7 [M+H]⁺.

### Step 8: Preparation of 2-((2S)-4-(7-(8-chloronaphthalen-1-yl)-5-methyl-2-((tetrahydro-1H-pyrrolizin-7a(5H)-yl)methoxy)-7,8-dihydro-5H-pyrano[4,3-d]pyrimidin-4-yl)piperazin-2-yl)acetonitrile

To a mixture of tert-butyl (2S)-4-(7-(8-chloronaphthalen-1-yl)-5-methyl-2-((tetrahydro-1H-pyrrolizin-7a(5H)-yl)methoxy)-7,8-dihydro-5H-pyrano[4,3-d]pyrimidin-4-yl)-2-(cyanomethyl) piperazine-1-carboxylate (300 mg, 0.44 mmol) in 2,2,2-Trifluoroethanol (5 mL) was added TMSCI (242 mg, 2.22 mmol). The reaction was stirred at 20° C. for 3 hr. and then concentrated to give the titled product (250 mg, crude) as a yellow solid. LC/MS: 572.7 [M+H]⁺.

### Step 9: Preparation of 2-((2S)-4-(7-(8-chloronaphthalen-1-yl)-5-methyl-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (compound 59)

To a solution of 2-[(2S)-4-[7-(8-chloronaphthalen-1-yl)-2-(hexahydropyrrolizin-7a-ylmethoxy)-5-methyl-5H,7H,8H-pyrano[4,3-d]pyrimidin-4-yl]piperazin-2-yl]acetonitrile (50 mg, 0.09 mmol), 2-fluoroprop-2-enoic acid (24 mg, 0.26 mmol) and TEA (53 mg, 0.52 mmol) in DCM (10 mL) was added T₃P (139 mg, 0.44 mmol, 50% in EA). The mixture was stirred at 20° C. for 36 h. The mixture was concentrated and purified by prep-HPLC (ACN/H₂O (0.1% TFA) 65/30 to 45/55) to afford the desired product (14 mg, 25%) as a white solid. LC/MS: 644.6 [M+H]⁺.

¹H NMR (400 MHz, MeOD) δ 8.10 (d, J=7.2 Hz, 1H), 8.00-7.90 (m, 2H), 7.72-7.57 (m, 2H), 7.52-7.39 (m, 1H), 6.56-6.44 (m, 1H), 5.50-5.28 (m, 3H), 5.01-4.81 (m, 1H), 4.73-4.49 (m, 2H), 4.47-4.01 (m, 3H), 3.96-3.85 (m, 1H), 3.74-3.65 (m, 2H), 3.61-3.43 (m, 2H), 3.32-3.12 (m, 3H), 3.01-2.77 (m, 3H), 2.44-2.00 (m, 8H), 1.60-1.49 (m, 3H).

### Example 7

### 2-((S)-1-(2-fluoroacryloyl)-4-((5R,7R)-5-methyl-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 60) and 2-((S)-1-(2-fluoroacryloyl)-4-((5S,7S)-5-methyl-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 61)

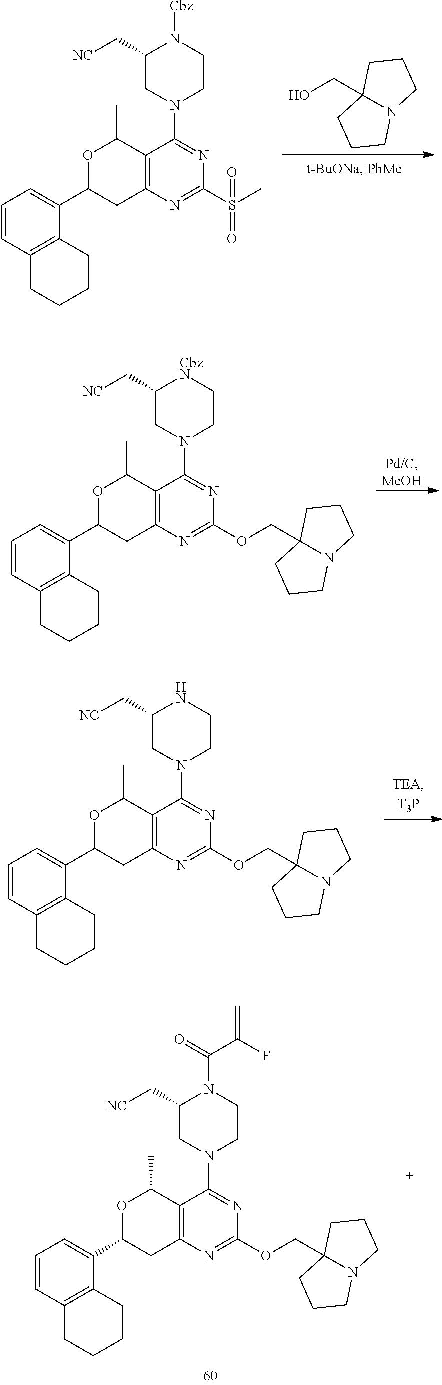

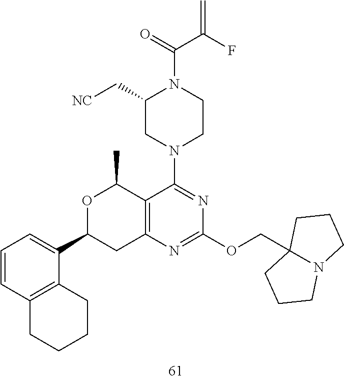 [SMILES: C=C(F)C(=O)N1CCN(c2nc(OCC34CCCN3CCC4)nc3c2[C@H](C)O[C@H](c2cccc4c2CCCC4)C3)C[C@@H]1CC#N]

Compounds 60 and 61 were prepared analogously with the procedure described for example 6 using commercially available 5,6,7,8-Tetrahydronaphthalene-1-carboxaldehyde.

2-((S)-1-(2-fluoroacryloyl)-4-((5R,7R)-5-methyl-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 60)

¹H NMR (400 MHz, CD₃OD) δ 7.31 (d, J=7.6 Hz, 1H), 7.16 (t, J=7.6 Hz, 1H), 7.07 (d, J=7.2 Hz, 1H), 5.45-5.27 (m, 3H), 5.08-5.03 (m, 1H), 4.99-4.93 (m, 1H), 4.89-4.85 (m, 1H), 4.84-4.79 (m, 1H), 4.63-4.58 (m, 1H), 4.22-4.04 (m, 1H), 3.93-3.87 (m, 1H), 3.74-3.67 (m, 2H), 3.63-3.55 (m, 1H), 3.48-3.45 (m, 1H), 3.30-3.25 (m, 1H), 3.19-3.11 (m, 1H), 3.09-2.67 (m, 9H), 2.37-2.11 (m, 8H), 1.90-1.79 (m, 4H), 1.44-1.37 (m, 3H).

2-((S)-1-(2-fluoroacryloyl)-4-((5S,7S)-5-methyl-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 61)

¹H NMR (400 MHz, CD₃OD) δ 7.31 (d, J=7.5 Hz, 1H), 7.16 (m, 1H), 7.09-7.05 (m, 1H), 5.46-5.28 (m, 3H), 5.12-5.05 (m, 1H), 4.88-4.79 (m, 1H), 4.71-4.57 (m, 2H), 4.49-4.38 (m, 1H), 4.28-4.13 (m, 1H), 4.10-4.02 (m, 1H), 3.98-3.90 (m, 1H), 3.78-3.64 (m, 3H), 3.31-3.26 (m, 1H), 3.11-2.58 (m, 10H), 2.40-2.06 (m, 8H), 1.90-1.77 (m, 4H), 1.50-1.45 (m, 3H).

### Example 8

### 2-((2S)-4-(7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (Compound 62), 2-((S)-4-((R)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (Compound 63) and 2-((S)-4-((S)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (Compound 64)

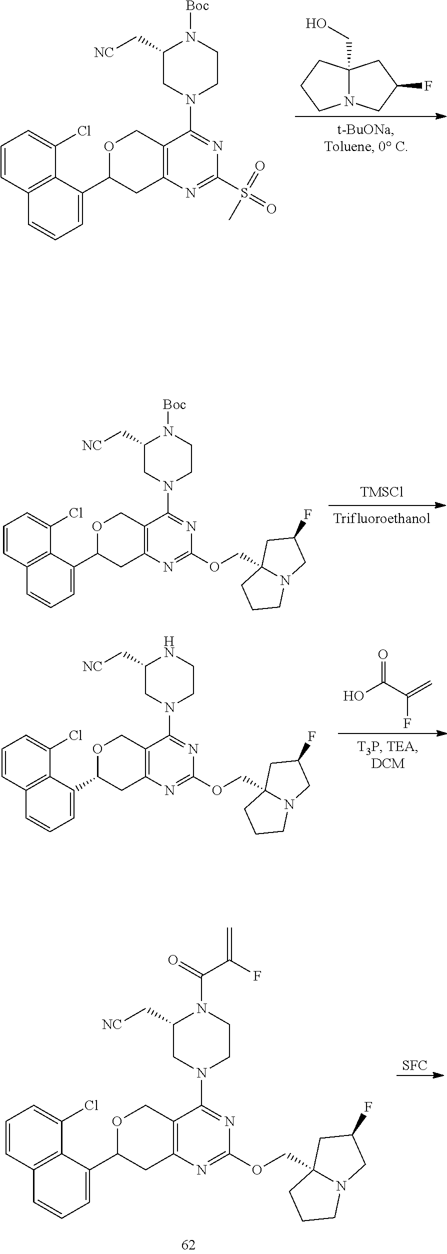

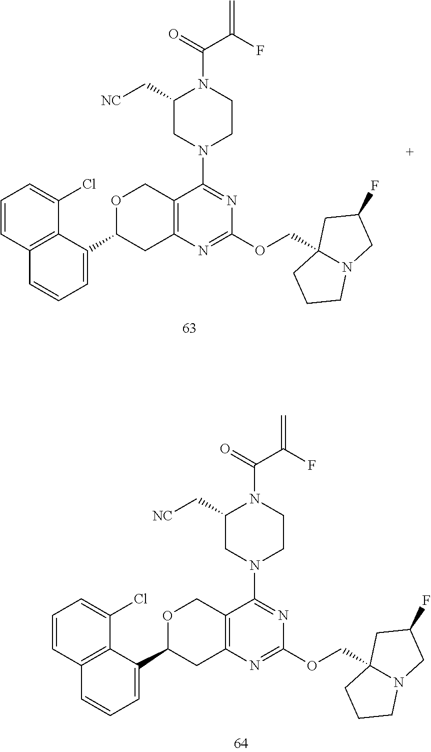 [SMILES: C=C(F)C(=O)N1CCN(c2nc(OC[C@@]34CCCN3C[C@H](F)C4)nc3c2CO[C@@H](c2cccc4cccc(Cl)c24)C3)C[C@@H]1CC#N ; C=C(F)C(=O)N1CCN(c2nc(OC[C@@]34CCCN3C[C@H](F)C4)nc3c2CO[C@H](c2cccc4cccc(Cl)c24)C3)C[C@@H]1CC#N]

### Step 1: Preparation of tert-butyl (2S)-4-(7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-2-(cyanomethyl)piperazine-1-carboxylate

To a mixture of tert-butyl (2S)-4-(7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate (200 mg, 0.33 mmol) and [(2R,7aS)-2-fluoro-hexahydropyrrolizin-7a-yl]methanol (79.7 mg, 0.5 mmol) in toluene (5 mL) was added t-BuONa (64.2 mg, 0.67 mmol). After stirring at 0° C. for 2 hr. the reaction was diluted with EA (20 mL). The organic layer was washed sat. NH₄Cl solution and brine. The organic layer was dried over Na₂SO₄, filtered and the filtrate was concentrated. The filtrate was concentrated and purified by Combi-Flash (eluted by DCM/MeOH=10/1) to give the titled product (140 mg, 62.6%) as a yellow solid. LC/MS: 676.7 [M+H]⁺.

### Step 2: Preparation of 2-((2S)-4-(7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)piperazin-2-yl)acetonitrile

To a mixture of tert-butyl (2S)-4-(7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-2-(cyanomethyl)piperazine-1-carboxylate (140 mg, 0.21 mmol) in 2,2,2-trifluoroethanol (2 mL) was added TMSCI (112 mg, 1 mmol). After stirring at 20° C. for 4 hr, the reaction was concentrated to give the product (100 mg, crude) as a yellow solid. LC/MS: 576.7 [M+H]⁺.

### Step 3: Preparation of 2-((2S)-4-(7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (Compound 62)

To a mixture of 2-[(2S)-4-(2-{[(2R,7aS)-2-fluoro-hexahydropyrrolizin-7a-yl]methoxy}-7-(8-chloronaphthalen-1-yl)-5H,7H,8H-pyrano[4,3-d]pyrimidin-4-yl)piperazin-2-yl]acetonitrile (100 mg, 0.17 mmol), 2-fluoroprop-2-enoic acid (46.8 mg, 0.5 mmol) and TEA(87.7 mg, 0.87 mmol) in DCM (10 mL) was added T₃P (551 mg, 0.87 mmol, 50% in EA). The reaction was stirred for 24 h at 20° C. The reaction mixture was concentrated and purified by Prep-HPLC using a gradient of 0.1% TFA/ACN from 80:20 to 40:60 to give the titled product (50 mg, 44%) as a white solid. LC/MS: 648.7 [M+H]⁺.

¹H NMR (400 MHz, MeOD) δ 8.04 (d, J=7.5 Hz, 1H), 7.96 (dd, J=7.9, 6.4 Hz, 2H), 7.72-7.67 (m, 1H), 7.62-7.60 (m, 1H), 7.49-7.44 (m, 1H), 6.57-6.48 (m, 1H), 5.59 (d, J=52.0 Hz, 1H), 5.44-5.32 (m, 2H), 5.20-5.19 (m, 1H), 5.07-4.96 (m, 1H), 4.90-4.81 (m, 1H), 4.80-4.52 (m, 2H), 4.39-4.19 (m, 2H), 4.10-3.78 (m, 4H), 3.76-3.52 (m, 2H), 3.47-3.32 (m, 2H), 3.30-3.11 (m, 1H), 3.11-2.75 (m, 3H), 2.75-2.54 (m, 2H), 2.50-2.14 (m, 4H).

The racemate sample of compound 62 (42 mg, 0.065 mmol) was separated by SFC (Column: CHIRALPAK OJ-H 250 mm 20 mm, 5 μm; Modifier: 40% MEOH (NH₄OH 0.2%); Total Flow: 40g/min) to give 2-((S)-4-((R)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (Compound 63, first peak, 13.2 mg, 31.1%) and 2-((S)-4-((S)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile (Compound 64, 13.3 mg, 31.3%). LC/MS: 648.7[M+H]⁺.

Compound 63:

¹H NMR (400 MHz, MeOD) δ 8.03 (d, J=7.5 Hz, 1H), 7.95-7.92 (m, 2H), 7.67 (d, J=8.2 Hz, 1H), 7.60-7.58 (m, 1H), 7.46-7.42 (m, 1H), 6.52-6.48 (m, 1H), 5.41-5.24 (m, 3H), 5.14-5.10 (m, 1H), 5.02-4.93 (m, 1H), 4.90-4.86 (m, 1H), 4.43-4.39 (m, 1H), 4.20-4.14 (m, 3H), 3.94-3.91 (m, 1H), 3.52-3.47 (m, 3H), 3.33-3.16 (m, 4H), 3.12-3.02 (m, 3H), 2.77-2.70 (m, 1H), 2.23-2.12 (m, 3H), 2.01-1.89 (m, 3H).

Compound 64:

¹H NMR (400 MHz, MeOD) δ 8.04 (d, J=7.5 Hz, 1H), 7.96-7.93 (m, 2H), 7.67 (d, J=8.2 Hz, 1H), 7.66-7.59 (m, 1H), 7.47-7.43 (m, 1H), 6.55-6.52 (m, 1H), 5.58 (d, J=52.0 Hz, 1H), 5.36-5.30 (m, 2H), 5.16-5.12 (m, 1H), 5.00-4.97 (m, 1H), 4.93-4.91 (m, 1H), 4.58-4.53 (m, 2H), 4.28-4.22 (m, 2H), 4.15-3.83 (m, 4H), 3.80-3.42 (m, 4H), 3.32-3.22 (m, 1H), 2.99-2.93 (m, 2H), 2.75-2.53 (m, 3H), 2.37-2.31 (m, 3H), 2.19-2.03 (m, 1H).

Compound 64 was also prepared with the following scheme and procedure

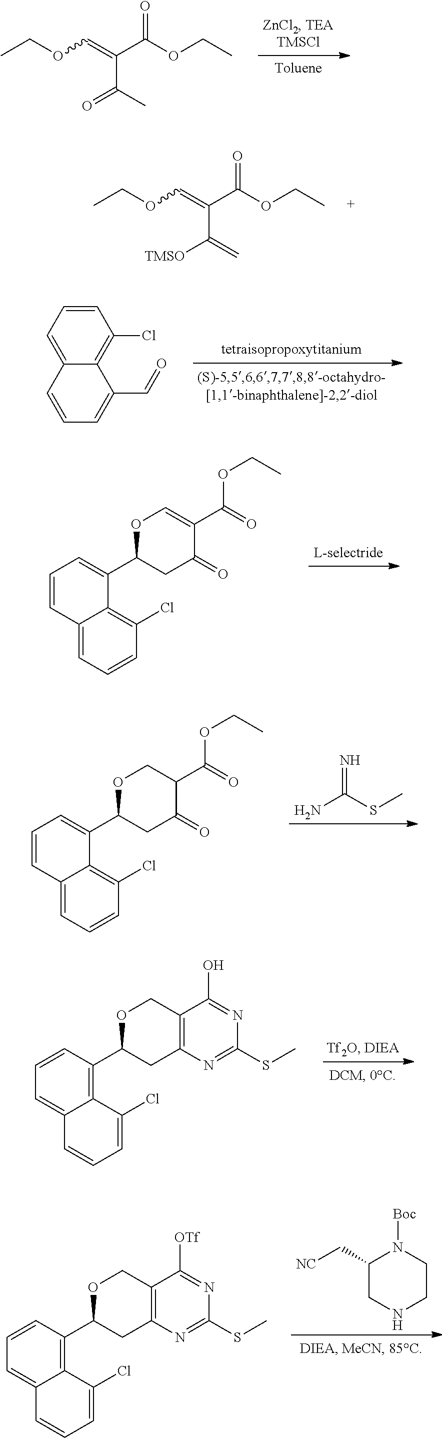

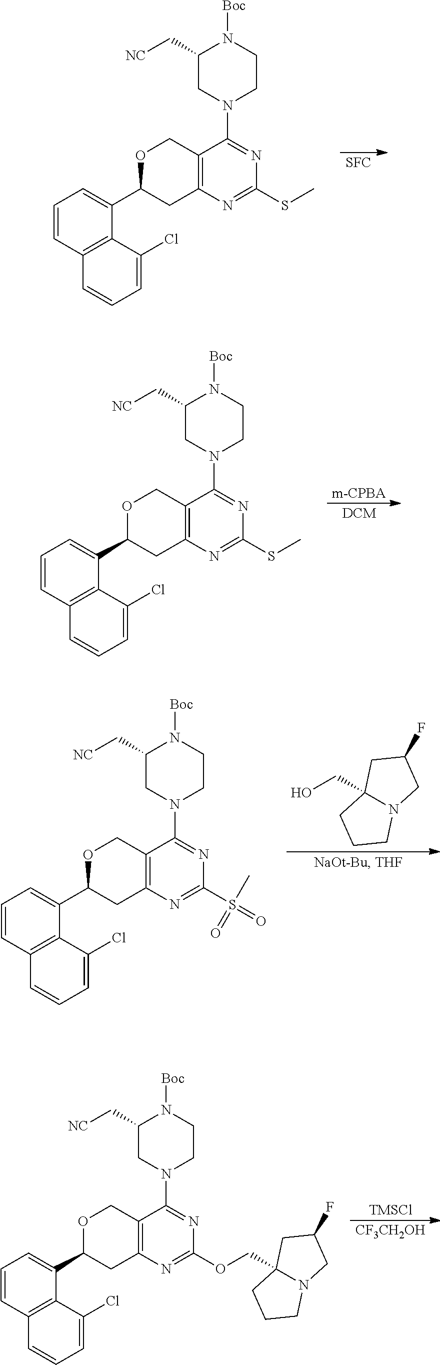

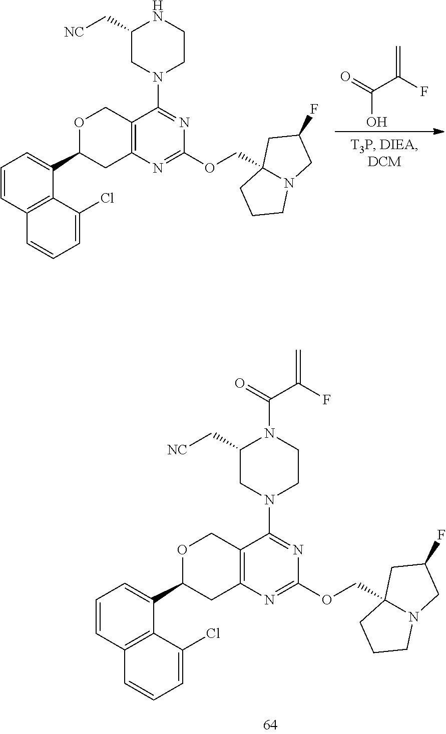

### Step 1: Preparation of ethyl 2-(ethoxymethylene)-3-((trimethylsilyl)oxy)but-3-enoate

ZnCl₂ (1.10 g, 8.06 mmol, 377 uL, 0.03 eq) was added to TEA (62.5 g, 618 mmol, 86.0 mL, 2.0 eq) at 25° C. slowly. After stirring at 25° C. for 1 hr, ethyl 2-(ethoxymethylene)-3-oxobutanoate (50.0 g, 269 mmol, 1.00 eq) in toluene (200 mL) was added, and then chloro(trimethyl)silane (58.4 g, 537 mmol, 68.2 mL, 2.00 eq) was added. The mixture was stirred at 40° C. for 12 hrs. The mixture was concentrated under reduced pressure to give the titled product which was used as is without further purification. ¹HNMR: (400 MHz, CDCl3) 6.91 (s, 1H), 4.46-4.48 (d, J=1.60 Hz, 1H), 4.32-4.38 (q, J=7.20 Hz, 2H), 4.05-4.14 (q, J=7.20 Hz, 2H), 2.44 (s, 2H), 1.38-1.42 (m, 6H), 0.33 (s, 9H)

### Step 2: Preparation of ethyl (S)-2-(8-chloronaphthalen-1-yl)-4-oxo-3,4-dihydro-2H-pyran-5-carboxylate

To a solution of 4A MS (60.0 g) and (S)-5,5,6,6,7,7,8,8′-octahydro-[1,1′-binaphthalene]-2,2′-diol (12.4 g, 42.0 mmol, 0.40 eq) in toluene (400 mL) was added tetraisopropoxytitanium (5.96 g, 21.0 mmol, 6.19 mL, 0.20 eq), and then stirred at 20° C. for 1 hr. 8-Chloro-1-naphthaldehyde (20.0 g, 105 mmol, 1.00 eq) was added to the mixture, then ethyl 2-(ethoxymethylene)-3-((trimethylsilyl)oxy)but-3-enoate (81.3 g, 315 mmol, 3.0 eq) was added to the mixture slowly at 60° C. The mixture was stirred at 60° C. for 12 hrs. The reaction mixture was filtered, and then diluted with EtOAc (550 mL). The organic layer was washed with aq. NaHCO₃ (450 mL×2) and brine (350 ml), dried over Na₂SO₄, filtered and concentrated under reduced pressure to dryness. The residue was purified with silica gel chromatography column by eluting with DCM: Ethyl acetate=1: 0-50: 1 to give the titled product (39.6 g, 72.8 mmol, 69.4% yield, 60.8% purity) as a yellow oil.

### Step 3: Preparation of ethyl (65)-6-(8-chloronaphthalen-1-yl)-4-oxotetrahydro-2H-pyran-3-carboxylate

To a solution of ethyl (S)-2-(8-chloronaphthalen-1-yl)-4-oxo-3,4-dihydro-2H-pyran-5-carboxylate (29.0 g, 87.7 mmol, 1.00 eq) in THF (750 mL) was added L-selectride (1 M, 96.4 mL, 1.10 eq) at -78° C. under N2 atmosphere. The mixture was stirred at -78° C. for 1 hr. The reaction mixture was poured into cold aq. NH₄Cl (800 mL), and then extracted with Ethyl acetate (500 mL*2). The combined organic layer was washed with brine (500 mL), dried over Na₂SO₄, filtered and concentrated under reduced pressure to give the titled product (36 g) which was used as is in the next step without further purification.

### Step 4: Preparation of (S)-7-(8-chloronaphthalen-1-yl)-2-(methylthio)-7,8-dihydro-5H-pyrano[4,3-d]pyrimidin-4-ol

To a mixture of ethyl (6S)-6-(8-chloronaphthalen-1-yl)-4-oxotetrahydro-2H-pyran-3-carboxylate (35.0 g, 105 mmol, 1 eq) in t-BuOH (900 mL) and H₂O (180 mL) was added 2-methylisothiourea (14.6 g, 105 mmol, 1.0 eq, 0.5 H₂SO₄) and Na₂CO₃ (89.2 g, 841 mmol, 8.0 eq) portion wise at 25° C. The mixture was stirred at 25° C. for 12 hrs and at 60° C. for 12 hrs under N₂. The mixture was filtered and the filtrate was concentrated. The residue was diluted with H₂O (300 mL), adjusted to pH=5 with AcOH, and extracted with DCM (500 mL*2). The combined organic layers were dried over Na₂SO₄, filtered and the filtrate was concentrated. The residue was triturated with MeCN (20.0 mL). The mother liquid was concentrated and purified with silica gel chromatography column by eluting with DCM: MeOH=1:0-200:1 to give the titled product (6.60 g, 15.3 mmol, 83.2% purity) as a yellow solid. LC-MS: 358.9 (M+H)⁺.

### Step 5: Preparation of (S)-7-(8-chloronaphthalen-1-yl)-2-(methylthio)-7,8-dihydro-5H-pyrano[4,3-d]pyrimidin-4-yl trifluoromethanesulfonate

To a solution of (S)-7-(8-chloronaphthalen-1-yl)-2-(methylthio)-7,8-dihydro-5H-pyrano[4,3-d]pyrimidin-4-ol (9.00 g, 25.1 mmol, 1 eq) in DCM (270 mL) was added DIEA (6.81 g, 52.67 mmol, 9.17 mL, 2.10 eq) and Tf₂O (13.0 g, 45.9 mmol, 7.57 mL, 1.83 eq) at 0° C. under N₂ atmosphere. The mixture was stirred at 0° C. for 1 hr. The reaction mixture was diluted with H₂O (200 mL) and extracted with DCM (150 mL*2). The combined organic layers were washed with brine (250 mL), dried over Na₂SO₄, filtered and concentrated under reduced pressure to give the titled product (13.0 g, crude) which was used in the next step without further purification. LCMS: 490.9 (M+H)⁺.

### Step 6: Preparation of tert-butyl (S)-4-((S)-7-(8-chloronaphthalen-1-yl)-2-(methylthio)-7,8-dihydro-5H-pyrano[4,3-d]pyrimidin-4-yl)-2-(cyanomethyl)piperazine-1-carboxylate

To a mixture of (S)-7-(8-chloronaphthalen-1-yl)-2-(methylthio)-7,8-dihydro-5H-pyrano[4,3-d]pyrimidin-4-yl trifluoromethanesulfonate (13.0 g, 26.5 mmol, 1.00 eq) and tert-butyl (S)-2-(cyanomethyl)piperazine-1-carboxylate (6.86 g, 30.45 mmol, 1.15 eq) in MeCN (250 mL) was added DIEA (11.0 g, 84.74 mmol, 14.76 mL, 3.20 eq) slowly. The reaction was stirred at 80° C. for 2 hr. The reaction mixture was concentrated under reduced pressure. The residue was diluted with EtOAc (250 mL) and washed with sat. NaHCO₃ solution (200 mL) and brine (150 mL). The organic layer was dried over Na₂SO₄, filtered and concentrated under reduced pressure. The residue was purified with silica gel column chromatography by eluting with Petroleum ether:Ethyl acetate=100:1-5:1 to give the titled product (7.72 g, 13.5 mmol, 51.0% yield, 80% ee) as a yellow solid. LCMS: 566.1 (M+H)⁺. ¹HNMR: (400 MHz, CDCl₃) 7.90-8.01 (d, J=7.20 Hz, 1H), 7.77-7.89 (t, J=8.80 Hz, 2H), 7.65-7.50 (m, 2H), 7.33-7.42 (t, J=8.00 Hz, 2H), 6.48-6.56 (dd, J=3.20, 10.8 Hz, 1H), 5.05-4.75 (m, 2H), 4.62 (s, 1H), 4.05 (s, 1H), 3.85-3.98 (d, J=13.6 Hz, 1H), 3.79-3.52 (m, 2H), 3.38-3.49 (dd, J=4.00, 14.0 Hz, 1H), 3.31 (s, 1H), 2.95-3.08 (dt, J=3.60, 12.4 Hz, 1H), 2.80- 2.94 (dd, J=10.8, 18.0 Hz, 1H), 2.75-2.60 (m, 2H), 2.52 (s, 3H), 1.52 (s, 9H).

tert-Butyl (S)-4-((S)-7-(8-chloronaphthalen-1-yl)-2-(methylthio)-7,8-dihydro-5H-pyrano[4,3-d]pyrimidin-4-yl)-2-(cyanomethyl)piperazine-1-carboxylate (7.50 g, 80% ee) was purified by SFC (column: DAICEL CHIRALPAK AD (250 mm*50 mm, 10 um); mobile phase: [0.1% NH₃H₂O ETOH]; B %: 60%-60%, 7.3; 120 min) to give the titled product (6.00 g, 79.3% yield, 100% ee) as a yellow solid. LCMS: 566.1 (M+H)⁺.

¹HNMR: (400 MHz, CDCl3) δ 7.94-8.02 (d, J=7.20 Hz, 1H), 7.78-7.89 (t, J=8.80 Hz, 2H), 7.65-7.49 (m, 2H), 7.33-7.41 (t, J=8.00 Hz, 1H), 6.45-6.40 (dd, J=3.20, 11.2 Hz, 1H), 5.07-4.75 (m, 2H), 4.62 (s, 1H), 4.13-3.84 (m, 2H), 3.79-3.52 (m, 2H), 3.38-3.49 (dd, J=3.60, 14.0 Hz, 1H), 3.31 (br s, 1H), 2.95-3.07 (dt, J=3.60, 12.4 Hz, 1H), 2.81-2.94 (dd, J=11.2, 18.4 Hz, 1H), 2.77-2.58 (m, 2H), 2.52 (s, 3H), 1.52 (s, 9H)

### Step 7: Preparation of tert-butyl (S)-4-((S)-7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate

To a mixture of tert-Butyl (S)-4-((S)-7-(8-chloronaphthalen-1-yl)-2-(methylthio)-7,8-dihydro-5H-pyrano[4,3-d]pyrimidin-4-yl)-2-(cyanomethyl)piperazine-1-carboxylate (3.00 g, 5.30 mmol, 1 eq) in DCM (120 mL) was added m-CPBA (3.23 g, 15.9 mmol, 85% purity, 3 eq) portion wise. The reaction was stirred at 25° C. for 1 hr. The reaction mixture was diluted with DCM (200 mL). The organic layer was washed with sat. Na₂S₂O₃ solution (100 mL), sat. NaHCO₃ solution (150 mL) and brine (150 mL). The organic layer was dried over Na₂SO₄, filtered and the filtrate was concentrated to give the titled product (4.20 g, crude) which was used in the next step without further purification. LCMS: 598.3 MS (M+H)⁺.

### Step 8: Preparation of tert-butyl (S)-4-((S)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-2-(cyanomethyl)piperazine-1-carboxylate

To a mixture of tert-butyl (S)-4-((S)-7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate (4.20 g, 7.02 mmol, 1 eq) and ((2R,7aS)-2-fluorotetrahydro-1H-pyrrolizin-7a(5H)-yl)methanol (2.24 g, 14.0 mmol, 2 eq) in toluene (100 mL) was added t-BuONa (1.01 g, 10.5 mmol, 1.5 eq). The reaction was stirred at 25° C. for 2 hrs. The reaction was quenched with sat. NH₄Cl solution (100 mL) and extracted with EtOAc (100 mL*3). The organic layer was washed with brine (100 mL) and dried over Na₂SO₄, filtered and the filtrate was concentrated to give the titled product (4.70 g) which was used in the next step without further purification. LCMS: 677.2 MS (M+H)⁺.

### Step 9: Preparation of 2-((S)-4-((S)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)piperazin-2-yl)acetonitrile

To a solution of tert-butyl (S)-4-((S)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-2-(cyanomethyl)piperazine-1-carboxylate (4.70 g, 6.94 mmol, 1 eq) in 2,2,2-trifluoroethanol (80.0 mL) was added chloro(trimethyl)silane (2.26 g, 20.8 mmol, 2.64 mL, 3 eq) slowly. The reaction was stirred at 25° C. for 1 hr. The reaction mixture was concentrated under reduced pressure. The residue was dissolved in DCM (100 mL) and extracted with NaHCO₃ (sat. aq. 80.0 mL*2). The organic layer was washed with brine (80.0 mL), dried over Na₂SO₄, filtered and concentrated under reduced pressure to give the titled product (4.20 g, crude) which was used as is in the next step without further purification. LCMS: 577.3MS (M+H)⁺.

### Step 10: Preparation of 2-((S)-4-((S)-7-(8-chloronaphthalen-1-yl)-2-(((2R,7aS)-2-fluorotetrahydro-1 H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile

To a solution of 2-((S)-4-((S)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)piperazin-2-yl)acetonitrile (4.20 g, 7.28 mmol, 1 eq) and 2-fluoroprop-2-enoic acid (983 mg, 10.9 mmol, 1.5 eq) in DCM (80 mL) was added DIEA (2.82 g, 21.8 mmol, 3.80 mL, 3 eq) and T₃P (9.26 g, 14.56 mmol, 8.66 mL, 50% purity, 2 eq). After stirring at 25° C. for 2 hr, the reaction mixture was diluted with DCM (100 mL) and washed with NaHCO₃ (sat. aq. 80 mL*2). The organic layer was washed with brine (80.0 mL), dried over Na₂SO₄, filtered and concentrated under reduced pressure to dryness. The crude product was purified by reversed-phase HPLC (column: Phenomenex Luna C₁₈ (250*80 mm*15 um); mobile phase: [water (FA)-ACN]; B %: 30%-60%,20 min) to give the titled product (3.00 g, 4.62 mmol) as a yellow solid. LCMS: 649.4 (M+1)⁺.

¹HNMR: (400 MHz, CDCl₃) δ 7.90-8.00 (d, J=7.20 Hz, 1H), 7.75-7.88 (dd, J=8.40, 9.20 Hz, 2H), 7.65-7.49 (m, 2H), 7.30-7.40 (t, J=7.60 Hz, 1H), 6.40-6.55 (dd, J=3.20, 10.8 Hz, 1H), 5.64-5.09 (m, 4H), 4.90-5.05 (d, J=13.6 Hz, 1H), 4.89-4.63 (m, 3H), 4.40-4.55 (d, J=11.2 Hz, 1H), 4.18-3.42 (m, 9H), 3.31-3.06 (m, 2H), 3.01-2.78 (m, 3H), 2.73-2.36 (m, 3H), 2.31-2.13 (m, 3H)

### Example 9

### Preparation of 2-((S)-1-(2-fluoroacryloyl)-4-((5R,7R)-5-methyl-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (compound 65) and 2-((S)-1-(2-fluoroacryloyl)-4-((5S,7S)-5-methyl-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (compound 66)

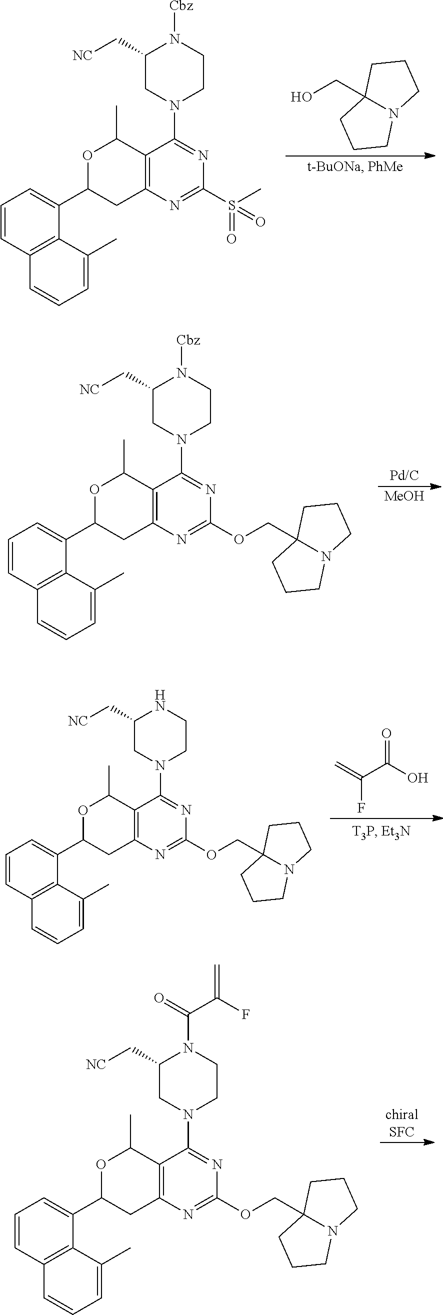

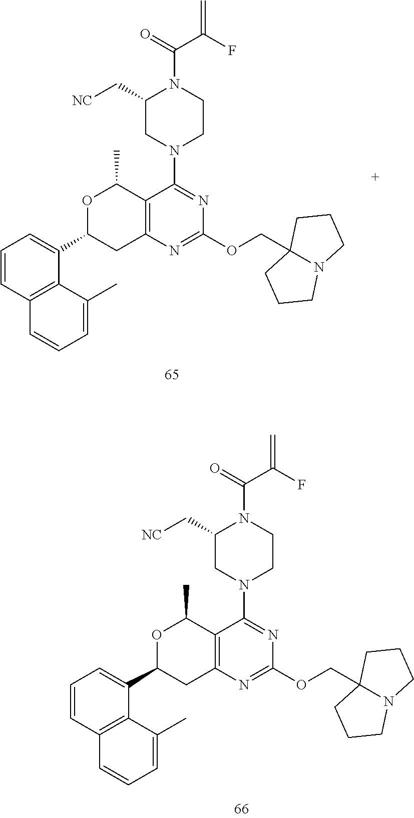 [SMILES: C=C(F)C(=O)N1CCN(c2nc(OCC34CCCN3CCC4)nc3c2[C@@H](C)O[C@@H](c2cccc4cccc(C)c24)C3)C[C@@H]1CC#N ; C=C(F)C(=O)N1CCN(c2nc(OCC34CCCN3CCC4)nc3c2[C@H](C)O[C@H](c2cccc4cccc(C)c24)C3)C[C@@H]1CC#N]

Compounds 65 and 66 were prepared analogously with the procedure as described in Example 5.

2-((S)-1-(2-fluoroacryloyl)-4-((5R,7R)-5-methyl-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (compound 65)

¹H NMR (400 MHz, CD₃OD) δ 7.94 (d, J=7.3 Hz, 1H), 7.88 (d, J=8.0 Hz, 1H), 7.81-7.76 (m, 1H), 7.59-7.50 (m, 1H), 7.42-7.35 (m, 2H), 6.06-6.04 (m, 1H), 5.50-5.31 (m, 3H), 5.08-5.04 (m, 1H), 4.68-4.65 (m, 1H), 4.24-4.11 (m, 1H), 4.00-3.98 (m, 1H), 3.75-3.62 (m, 3H), 3.52-3.50 (m, 1H), 3.42-3.24 (m, 5H), 3.17-3.15 (m, 2H), 3.07-2.96 (m, 2H), 2.90 (s, 3H), 2.39-2.32 (m, 2H), 2.22-2.15 (m, 4H), 2.14-2.07 (m, 2H), 1.50-1.46 (m, 3H).

2-((S)-1-(2-fluoroacryloyl)-4-((5S,7S)-5-methyl-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (compound 66)

¹H NMR (400 MHz, CD₃OD) δ 7.95 (d, J=7.5 Hz, 1H), 7.87 (d, J=8.0 Hz, 1H), 7.81-7.76 (m, 1H), 7.52 (t, J=7.7 Hz, 1H), 7.37 (d, J=5.6 Hz, 2H), 6.05-6.02 (m, 1H), 5.48-5.30 (m, 3H), 4.70-4.46 (m, 3H), 4.45-4.14 (m, 3H), 4.09-4.02 (m, 1H), 3.87-3.84 (m, 2H), 3.73-3.65 (m, 2H), 3.32-3.24 (m, 2H), 3.15-3.11 (m, 1H), 3.06-2.91 (m, 2H), 2.88-2.86 (m, 4H), 2.38-2.08 (m, 8H), 1.58-1.56 (m, 3H).

### Example 10

### Preparation of 2-((2S)-1-(2-fluoroacryloyl)-4-(2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 67), 2-((S)-1-(2-fluoroacryloyl)-4-((R)-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 68) and 2-((S)-1-(2-fluoroacryloyl)-4-((S)-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 69)

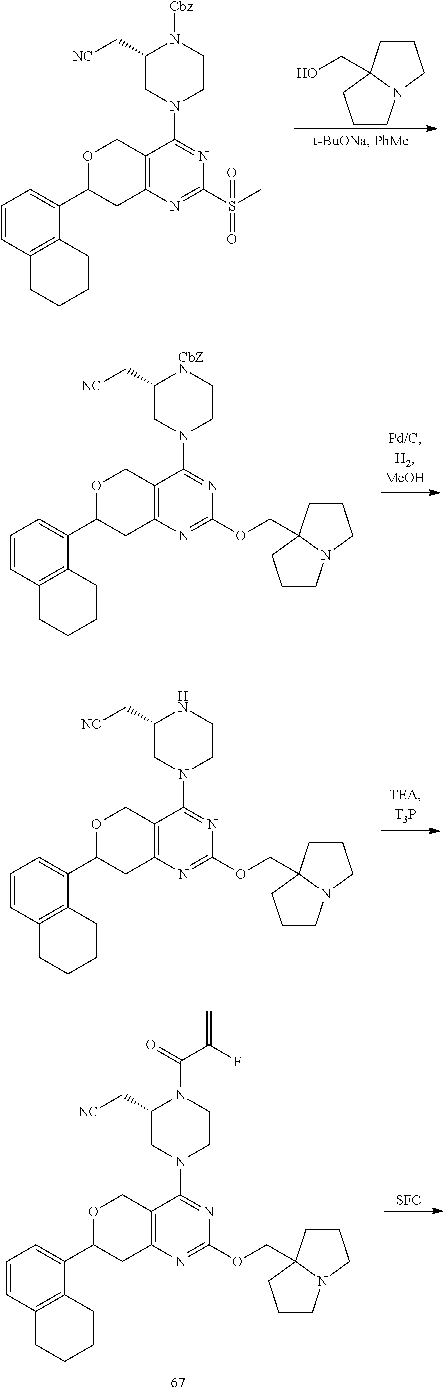

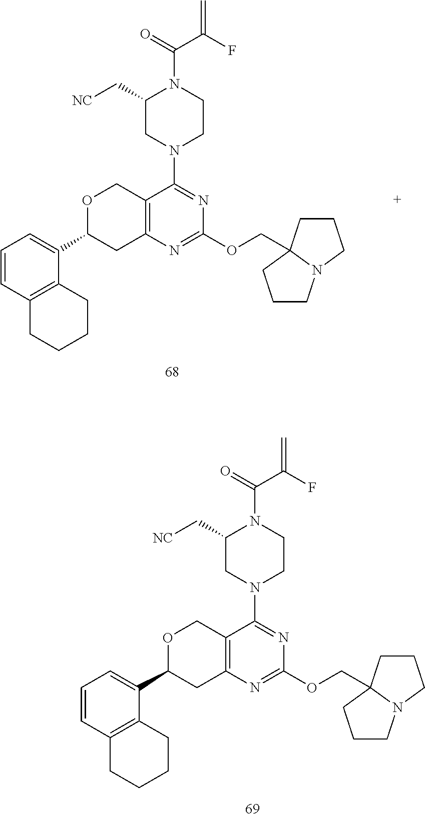 [SMILES: C=C(F)C(=O)N1CCN(c2nc(OCC34CCCN3CCC4)nc3c2CO[C@@H](c2cccc4c2CCCC4)C3)C[C@@H]1CC#N ; "+" ; C=C(F)C(=O)N1CCN(c2nc(OCC34CCCN3CCC4)nc3c2CO[C@H](c2cccc4c2CCCC4)C3)C[C@@H]1CC#N]

Compounds 67, 68 and 69 were prepared analogously with the procedure described in Example 5.

2-((2S)-1-(2-fluoroacryloyl)-4-(2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 67)

LC/MS: 600.8[M+H]⁺

¹H NMR (400 MHz, CD₃OD) δ 7.29-7.25 (m, 1H), 7.14 (t, J=7.6 Hz, 1H), 7.09-7.04 (m, 1H), 5.44-5.31 (m, 2H), 5.15-4.97 (m, 3H), 4.96-4.91 (m, 1H), 4.87-4.78 (m, 1H), 4.72-4.66 (m, 1H), 4.60-4.57 (m, 1H), 4.32-4.24 (m, 1H), 4.10-4.02 (m, 1H), 3.93-3.91 (m, 1H), 3.69 (d, J=11.8 Hz, 2H), 3.55-3.43 (m, 1H), 3.42-3.35 (m, 1H), 3.30-3.27 (m, 1H), 3.14-3.01 (m, 2H), 3.01-2.71 (m, 7H), 2.37-2.07 (m, 8H), 1.92-1.78 (m, 4H).

2-((S)-1-(2-fluoroacryloyl)-4-((R)-2-((tetrahydro-1 H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 68)

¹H NMR (400 MHz, CDCl₃) δ 7.30-7.27 (m, 1H), 7.16 (t, J=7.6 Hz, 1H), 7.08-7.04 (m, 1H), 5.40 (d, J=47.9 Hz, 1H), 5.24 (dd, J=16.8, 3.6 Hz, 1H), 5.01-4.96 (m, 1H), 4.88-4.82 (m, 1H), 4.74-4.69 (m, 1H), 4.14 (s, 2H), 3.99-3.93 (m, 1H), 3.90-3.81 (m, 1H), 3.64-3.44 (m, 2H), 3.31-3.01 (m, 5H), 2.91-2.63 (m, 8H), 2.20-2.07 (m, 2H), 1.99-1.62 (m, 12H).

2-((S)-1-(2-fluoroacryloyl)-4-((S)-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile (compound 69)

¹H NMR (400 MHz, CDCl₃) δ 7.26-7.23 (m, 1H), 7.15 (t, J=7.6 Hz, 1H), 7.08-7.04 (m, 1H), 5.39 (d, J=47.1 Hz, 1H), 5.28-5.21 (m, 1H), 4.98-4.93 (m, 1H), 4.90-4.81 (m, 2H), 4.72-4.60 (m, 2H), 4.46 (d, J=11.6 Hz, 1H), 3.90 (s, 2H), 3.70-3.63 (m, 1H), 3.47-3.34 (m, 2H), 3.31-3.25 (m, 1H), 3.17-3.10 (m, 1H), 3.08-2.93 (m, 3H), 2.90-2.78 (m, 3H), 2.71-2.41 (m, 8H), 2.37-2.27 (m, 2H), 2.15-2.05 (m, 2H), 2.00-1.90 (m, 2H), 1.87-1.76 (m, 3H).

### Example 11

### 2-((2S)-4-(7-(8-chloronaphthalen-1-yl)-2-((1-((dimethylamino)methyl)cyclopropyl)methoxy)-7,8-dihydro-5H-pyrano[4,3-d]pyrimidin-4-yl)-1-(2-fluoroacryloyl)piperazin-2-yl) acetonitrile (Compound 70)

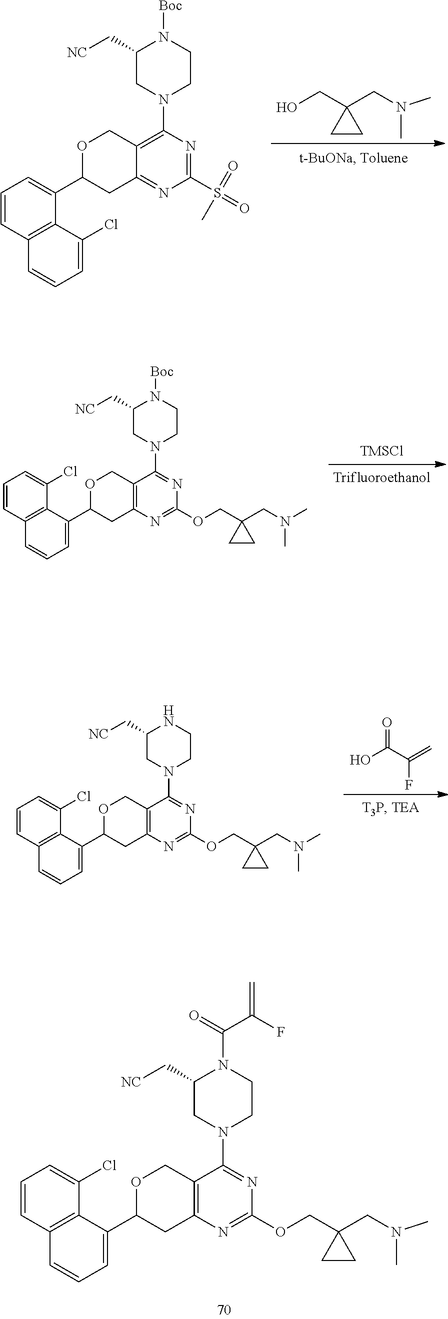

Compound 70 was prepared analogously with the procedure described in Example 8.

LC/MS: 618.8 [M+H]+.

¹H NMR (400 MHz, MeOD) δ 8.03 (d, J=7.5 Hz, 1H), 7.96-7.94 (m, 2H), 7.70-7.68 (m, 1H), 7.64-7.60 (m, 1H), 7.49-7.45 (m, 1H), 6.54-6.51 (m, 1H), 5.39-5.34 (m, 2H), 5.21-5.19 (m, 1H), 5.05-4.98 (m, 1H), 4.90-4.86 (m, 1H), 4.67-4.43 (m, 1H), 4.37-4.23 (m, 2H), 4.21-4.02 (m, 2H), 3.89-3.77 (m, 1H), 3.62-3.46 (m, 3H), 3.32-3.14 (m, 2H), 3.04-2.68 (m, 8H), 1.07-1.00 (m, 2H), 0.98-0.88 (m, 2H).

### Example 12

### Preparation of 2-((2S)-4-(7-(8-chloronaphthalen-1-yl)-2-((1-(pyrrolidin-1-ylmethyl)cyclopropyl)methoxy)-7,8-dihydro-5H-pyrano[4,3-d]pyrimidin-4-yl)-1-(2-fluoroacryloyl)piperazin-2-yl)acetonitrile formate (Compound 71); 2-((S)-4-((R)-7-(8-chloronaphthalen-1-yl)-2-((1-(pyrrolidin-1-ylmethyl)cyclopropyl)methoxy)-7,8-dihydro-5H-pyrano[4,3-d]pyrimidin-4-yl)-1-(2-fluoroacryloyl)piperazin-2-yl)acetonitrile (Compound 72); and 2-((S)-4-((S)-7-(8-chloronaphthalen-1-yl)-2-((1-(pyrrolidin-1-ylmethyl)cyclopropyl)methoxy)-7,8-dihydro-5H-pyrano[4,3-d]pyrimidin-4-yl)-1-(2-fluoroacryloyl)piperazin-2-yl)acetonitrile (Compound 73);

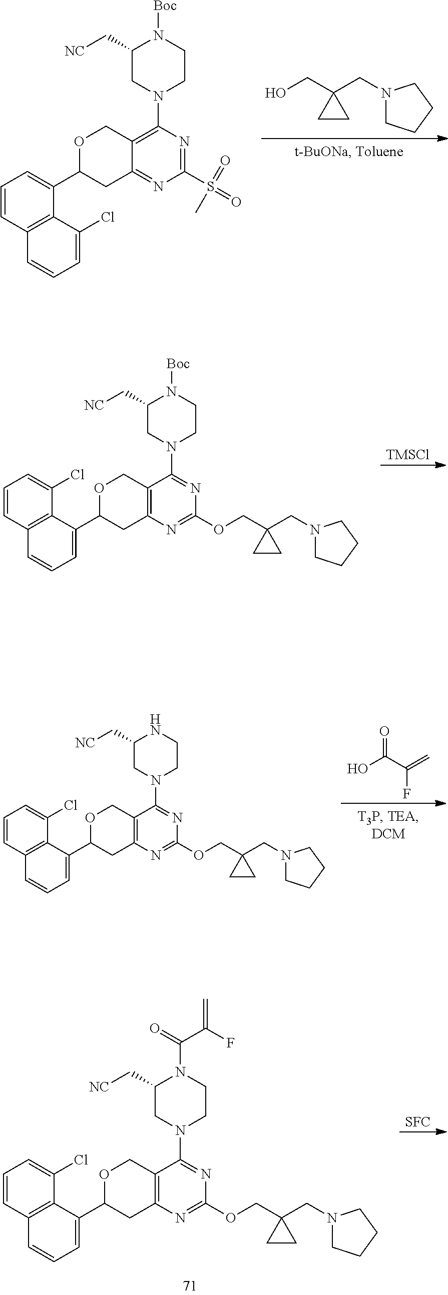

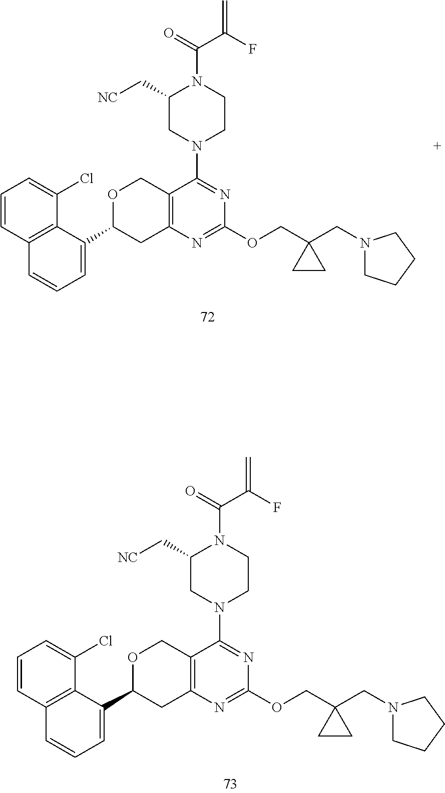 [SMILES: C=C(F)C(=O)N1CCN(c2nc(OCC3(CN4CCCC4)CC3)nc3c2CO[C@@H](c2cccc4cccc(Cl)c24)C3)C[C@@H]1CC#N ; C=C(F)C(=O)N1CCN(c2nc(OCC3(CN4CCCC4)CC3)nc3c2CO[C@H](c2cccc4cccc(Cl)c24)C3)C[C@@H]1CC#N]

Compounds 71, 72 and 73 were prepared analogously with the procedure described in Example 8.

Compound 71: LC/MS: 644.8[M+H]⁺

¹H NMR (400 MHz, MeOD) δ 8.05-8.02 (m, 1H), 7.94 (d, J=8.1 Hz, 2H), 7.68 (d, J=7.3 Hz, 1H), 7.61-7.59 (m, 1H), 7.45-7.43 (m, 1H), 6.56-6.49 (m, 1H), 5.41-5.36 (m, 1H), 5.34-5.26 (m, 1H), 5.14-5.12 (m, 1H), 5.01-4.92 (m, 1H), 4.69-4.51 (m, 1H), 4.45-4.43 (m, 1H), 4.32-4.31 (m, 2H), 4.25-4.13 (m, 2H), 4.05-3.90 (m, 1H), 3.82-3.62 (m, 1H), 3.59-3.47 (m, 2H), 3.43-3.36 (m, 1H), 3.29-2.85 (m, 6H), 2.77-2.68 (m, 1H), 2.01-1.98 (m, 4H), 1.08-0.82 (m, 4H).

The racemate sample of compound 71 (24 mg) was separated by SFC (Column: CHIRALPAK OJ-H 250 mm 20 mm, 5 μm; Modifier: 40% MEOH (NH₄OH 0.2%); Total Flow: 40 g/min) and suitable fractions were pooled and lyophilized to give Compound 72 (first peak; 6.3 mg, 26.3%) and Compound 73 (second peak; 6.5 mg, 27.1%).

Compound 72: LC/MS: 644.7[M+H]⁺.

¹H NMR (400 MHz, MeOD) δ 8.04 (d, J=7.3 Hz, 1H), 7.96-7.94 (m, 2H), 7.69 (dd, J=7.4, 1.2 Hz, 1H), 7.64-7.60 (m, 1H), 7.49-7.45 (m, 1H), 6.53-6.49 (m, 1H), 5.45-5.37 (m, 1H), 5.36-5.30 (m, 1H), 5.18-5.17 (m, 1H), 4.99-4.95 (m, 1H), 4.80-4.71 (m, 2H), 4.62-4.45 (m, 2H), 4.38-4.37 (m, 1H), 4.00-3.98 (m, 1H), 3.90-3.80 (m, 2H), 3.60-3.47 (m, 2H), 3.45-3.35 (m, 2H), 3.23-2.93 (m, 6H), 2.87-2.78 (m, 1H), 2.20-2.05 (m, 4H), 1.02-0.84 (m, 4H).

Compound 73:

¹H NMR (400 MHz, MeOD) δ 8.00 (d, J=7.4 Hz, 1H), 7.93-7.90 (m, 2H), 7.64 (d, J=6.6 Hz, 1H), 7.60-7.57 (m, 1H), 7.47-7.41 (m, 1H), 6.50-6.48 (m, 1H), 5.32-5.22 (m, 2H), 5.12-5.10 (m, 1H), 4.96-4.92 (m, 1H), 4.29 (s, 2H), 4.24-4.05 (m, 2H), 3.96-3.94 (m, 1H), 3.80-3.65 (m, 2H), 3.54-3.45 (m, 2H), 3.37-3.30 (m, 2H), 3.26-3.21 (m, 1H), 3.20-3.07 (m, 3H), 3.05-2.79 (m, 3H), 2.77-2.73 (m, 1H), 2.15-2.07 (m, 4H), 0.98-0.85 (m, 4H).

### Testing of Compounds for KRAS (KRAS G12C) Activity

### Example 13

### Gel Shift Assay to Determine the Activity of Exemplary Compounds in Modifying Mutant KRAS (KRAS G12C) and Wild Type KRAS

0.5 μM compounds were incubated with 1 μM GDP-bound untagged KRAS (G12C or wild type) proteins at 25° C. for 5 minutes in 40 μl reaction buffer (50 mM Tris, pH 7.5, 100 mM NaCl, 1 mM MgCl₂, 1 mM DTT). The reactions were quenched with 10 μI SDS-PAGE sample loading buffer (250 mM Tris-HCl, pH 6.8, 10% SDS, 0.5% bromophenol blue, 50% glycerol and 50 mM DTT) and then analyzed via SDS-PAGE using 4%-20% gradient polyacrylamide gel followed by Coomassie blue stain.

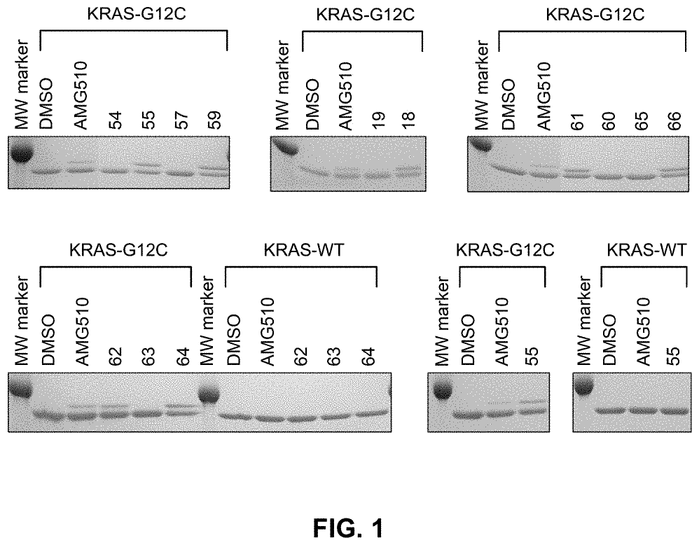

FIG. 1 illustrates mobility change of KRAS-G12C mutant or wild type proteins on SDS-PAGE after 5 minutes of coincubation at 25° C. with exemplary compounds of the present disclosure, indicative of covalent cysteine conjugation.

Table 3 summarizes molecular weight shift of KRAS-G12C mutant on SDS-PAGE after 5 mins of coincubation at 25° C. with exemplary compounds of the present disclosure, indicative of covalent cysteine conjugation.

TABLE 3
Molecular weight shift of KRAS-G12C mutant proteins on
SDS-PAGE after coincubation with exemplary compounds

| Compound # | KRAS-G12C molecular weight shift |
|---|---|
| 18 | Yes |
| 19 | No |
| 53 | Yes |
| 54 | No |
| 55 | Yes |
| 56 | Yes |
| 57 | No |
| 58 | Yes |
| 59 | Yes |
| 60 | No |
| 61 | Yes |
| 62 | Yes |
| 63 | No |
| 64 | Yes |
| 65 | No |
| 66 | Yes |
| 67 | Yes |
| 68 | No |
| 69 | Yes |
| 70 | Yes |
| 71 | Yes |
| 72 | No |
| 73 | Yes |

### Example 14

### TR-FRET Assay to Determine the Activity of the Disclosed Compounds in Inhibiting KRAS-GDP to KRAS-GTP Exchange Induced by SOS1

GDP form of 6*His tagged KRAS-G12C was diluted to 40 nM and incubated with 8 nM LanthaScreen™ Elite Terbium-anti-HIS Antibody (Thermo, catalog number PV5863) in reaction buffer containing 20 mM Tris, pH 7.5, 100 mM NaCl, 1 mM DTT, 0.1% Tween20, 0.05% BSA, 2% DMSO, 1 mM MgCl₂. After 1 hour-incubation at 4° C., proteins were placed in 384 well plate (PerkinElmer, PROXIPLATE-384 PLUS). Then compounds with different concentrations were added to the proteins. BODIPY labeled GTP (Thermo, catalog number G12411) and SOS-1 (or reaction buffer as control) were then added to reactions at 200 nM and 1 μM to trigger the exchange of GDP to BODIPY-GTP. TR-FRET emission signals were determined at 520 nm and 620 nm on a Envision® Multilabel Plate Reader (PerkinElmer) using a 337 nm laser as light source. All data were analyzed and plotted using Graph Pad Prism software (version 8.0.1). Raw TR-FRET data were converted to percentage of inhibition (relative to DMSO) using the following equations:

a) Emission ratio=Em520/Em620

b) For a given test compound concentration X:

Signal (X)=Emission ratio (SOS-1 & GTP)—Emission ratio (buffer & GTP)

c) Percentage of inhibition at concentration X=[1−Signal(X)/Signal (DMSO)]*100%

The 1050 values were determined by nonlinear regression of plots of [inhibitor] vs. percentage of inhibition with variable slope. Table 4 summarizes inhibition of SOS1-assisted GDP/GTP exchanging activity of KRAS-G12C mutant with exemplary compounds of the present disclosure.

TABLE 4
Inhibition of SOS1-assisted GDP/GTP exchanging activity of KRAS-
G12C mutant with exemplary compounds of the present disclosure

| Compound # | GDP/GTP exchange IC50 (uM) |
|---|---|
| 1 | 0.698 |
| 2 | 0.077 |
| 3 | 0.029 |
| 4 | 0.049 |
| 5 | 2.145 |
| 6 | 7.131 |
| 7 | 0.019 |
| 8 | 0.238 |
| 9 | 0.001 |
| 10 | 13.9 |
| 18 | 0.078 |
| 19 | 80.8 |
| 53 | 0.045 |
| 54 | 2.35 |
| 55 | 0.022 |
| 56 | 0.070 |
| 57 | 4.86 |
| 58 | 0.019 |
| 59 | 0.060 |
| 60 | 4.20 |
| 61 | 0.078 |
| 62 | 0.040 |
| 63 | 6.93 |
| 64 | 0.020 |
| 65 | 11.6 |
| 66 | 0.030 |
| 67 | 0.036 |
| 68 | 2.54 |
| 69 | 0.020 |
| 70 | 0.303 |
| 71 | 0.209 |
| 72 | 11.6 |
| 73 | 0.112 |

### Example 15

### pERK and ERK Western Blot Analysis

MIA PaCa-2 (ATCC), NCI-H358 (ATCC), NCI-H23 (ATCC), SW837 and A549 (ATCC) cells were plated in 24-well plates at 2×10⁵cells/well in RPMI growth medium containing 10% FBS and 1× Penicillin Streptomycin. They were then incubated at 37° C. overnight. The following day, the test compound was administered to the cells by using 1000× compound stock solution prepared in DMSO at various concentrations. After administration of the compound, the cells were then incubated at 37° C. for 4 hours. Upon completion, the cells were washed with PBS and protein was collected in Laemmli sample buffer (1×; VWR International). Proteins in cell lysate were separated by SDS-PAGE and transferred to Odyssey nitrocellulose membranes (Licor) with iblot® dry blotting transfer system (ThermoFisher). Nonspecific binding was blocked by incubating membranes with Intercept Blocking Buffer (Licor) for 1 hour at room temperature with gentle shaking. The membranes were then incubated overnight at 4° C. with primary antibodies rabbit anti-phospho p44/42 MAPK (Erk1/2) (1:2,000, Cell Signaling, 4370) and mouse anti-p44/42 MAPK (Erk1/2) (1:1,000, Cell Signaling, 4696) diluted in Intercept Blocking Buffer containing 0.1% Tween 20. After washing 3 times with TBS-T, the membranes were incubated with IRDye® 800CW goat anti-rabbit IgG (1:20,000, Licor) or IRDye® 680CW goat anti-mouse IgG (1:20,000, Licor) for 1 hour. After TBS-T washes, membranes were rinsed in TBS and scanned on Odyssey® CLx Imaging System (Licor). The bands were quantified using Image Studio™ Software (Licor).

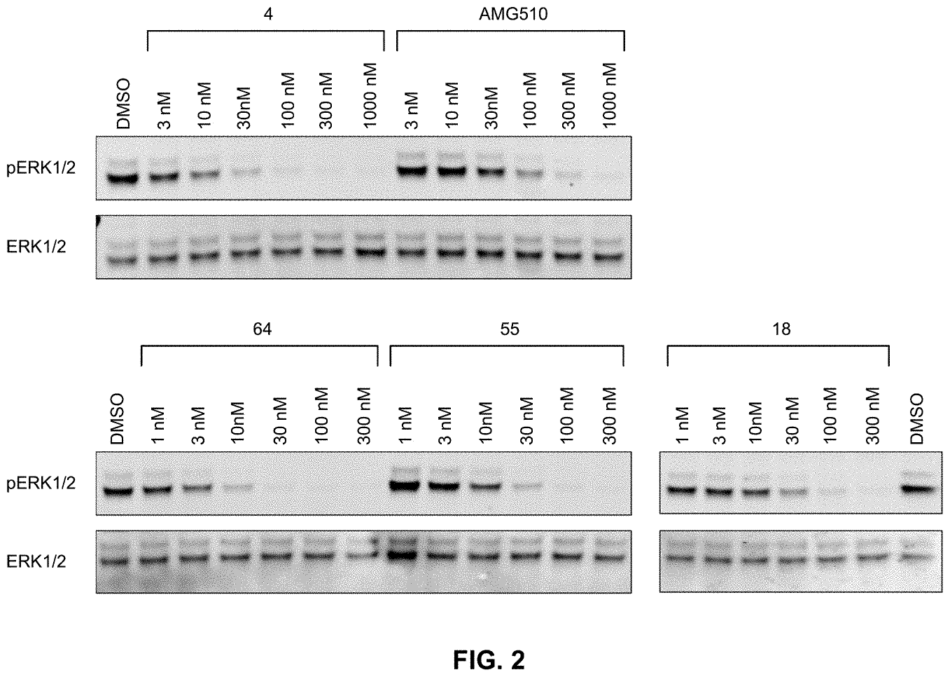

FIG. 2 illustrates phospho-ERK1/2 (Thr202/Tyr204) inhibition by exemplary compounds 4, 18, 55, and 64 of the present disclosure in a MIA PaCa-2 cell line 4 hours after incubation.

Table 5 summarizes phospho-ERK1/2 (Thr202/Tyr204) inhibition by exemplary compounds of the present disclosure in a MIA PaCa-2 cell line 4 hours after incubation. A: >0.1 nM and <1 nM; B: >=1 nM and <10 nM; C: >=10 nM and <100 nM; D: >100 nM.

TABLE 5
Phospho-ERK1/2 (Thr202/Tyr204) inhibition
by compounds in a MIA PaCa-2 cell line

| Compound # | 2D pERK IC50 (nM) |
|---|---|
| 1 | D |
| 2 | C |
| 3 | B |
| 4 | B |
| 5 | D |
| 6 | D |
| 7 | A |
| 8 | B |
| 9 | C |
| 10 | D |
| 18 | C |
| 19 | D |
| 53 | B |
| 54 | D |
| 55 | B |
| 56 | C |
| 57 | D |
| 58 | C |
| 59 | C |
| 60 | D |
| 61 | C |
| 62 | B |
| 63 | D |
| 64 | B |
| 65 | D |
| 66 | C |
| 67 | C |
| 68 | D |
| 69 | C |
| 70 | C |
| 71 | C |
| 72 | D |
| 73 | C |

TABLE 6
Phospho-ERK1/2 (Thr202/Tyr204) inhibition by compounds
18, 55, and 64 in multiple cell lines.

| | | pERK IC50 (nM) | | |
|---|---|---|---|---|
| | | Compound | Compound | Compound |
| Cell Lines | KRAS status | 64 | 55 | 18 |
| MiaPaCa2 | G12C | 2.4 | 10.1 | 21.9 |
| NCI-H23 | G12C | 1.7 | 6.4 | N/A |
| NCI-H358 | G12C | 2.5 | 12 | N/A |
| SW837 | G12C | 1.2 | 5.9 | N/A |
| A549 | G12S | >30,000 | >30,000 | N/A |

### Example 16

### 3D Cell Growth Assay to Determine the Activity of the Disclosed Compounds

MIA PaCa-2 (ATCC), NCI-H358 (ATCC), NCI-H23 (ATCC), SW837 and A549 (ATCC) cells were plated in round bottom 96-well spheroid microplates (Corning, 4520) at 3000 cells/well in 90 ul of RPMI growth medium containing 10% FBS and 1% Penicillin Streptomycin. Cells were incubated at 37° C. overnight. The following day, the test compound was administered to the cells by using 10× compound stock solution prepared in growth medium at various concentrations. After administration of the compound, cells were then incubated at 37° C. for 6 days. Before CellTiter-Glo assay, the plates were equilibrated at room temperature for approximately 10 minutes. 100 ul of CellTiter-Glo® Reagent (Promega, G7573) was added to each well. The plates were then incubated at room temperature for 10 minutes and luminescence was recorded by EnSpire plate reader (PerkinElmer). Table 6 illustrates growth inhibition (G150) by exemplary compounds 1-10 of the present disclosure in a MIA PaCa-2 cell line 6 days after administration in 3D cell culture. A: >0.1 nM and <1 nM; B: >=1 nM and <10 nM; C: >=10 nM and <100 nM; D: >100 nM.

TABLE 7
Growth inhibition (GI50) of compounds in a MIA PaCa-2 cell line

| Compound # | 3D growth inhibition GI50 (nM) |
|---|---|
| 1 | N/A |
| 2 | B |
| 3 | N/A |
| 4 | B |
| 5 | N/A |
| 6 | N/A |
| 7 | B |
| 8 | C |
| 9 | C |
| 10 | N/A |
| 18 | B |
| 19 | N/A |
| 53 | B |
| 54 | N/A |
| 55 | A |
| 56 | B |
| 57 | N/A |
| 58 | B |
| 59 | B |
| 60 | N/A |
| 61 | C |
| 62 | A |
| 63 | C |
| 64 | A |
| 65 | N/A |
| 66 | B |
| 67 | N/A |
| 68 | N/A |
| 69 | B |
| 70 | N/A |
| 71 | C |
| 72 | D |
| 73 | C |

TABLE 8
Growth inhibition (GI50) of compounds 18, 55 and
64 in multiple cell lines in 3D cell growth assay

| | | 3D growth inhibition GI50 (nM) | | |
|---|---|---|---|---|
| | | Compound | Compound | Compound |
| Cell lines | KRAS status | 64 | 55 | 18 |
| MiaPaCa2 | G12C | 0.3 | 0.8 | 7.3 |
| NCI-H23 | G12C | 2.1 | 8.4 | Not tested |
| NCI-H358 | G12C | 0.2 | 0.7 | Not tested |
| SW837 | G12C | 4.1 | 8.6 | Not tested |
| A549 | G12S | >300 | >300 | Not tested |

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description or examples.

1-64. (canceled)

65. A compound of Formula (1) or a tautomer, stereoisomer or a mixture of stereoisomers, or a pharmaceutically acceptable salt, or hydrate, or deuterated derivative thereof: 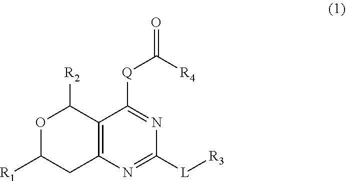
wherein:
R₁ is chosen from an aryl group, cycloalkyl, heterocycle, and heteroaryl, wherein each of the aryl group, cycloalkyl, heterocycle, and heteroaryl is independently substituted with 0, 1, 2, 3, or 4 R₅;
R₂ is chosen from hydrogen, C₁-C₄alkyl, and C₃-C₄cycloalkyl;
R₃ is chosen from hydrogen, C₁-C₆ alkyl, C₁-C₆ alkoxyalkyl, C₁-C₆ hydroxyalkyl, C₁-C₆ dihydroxyalkyl, C₁-C₆ dialkylaminoalkyl, C₃-C₆ cycloalkyl, C₃-C₆ heterocycle, C₃-C₆ heterocycloalkyl, aryl, and heteroaryl, each of which is independently substituted with 0, 1, 2, 3, or 4 R₆;
R₄ is chosen from a C₂-C₆ alkenyl and C₂-C₆ alkynyl, wherein each of the C₂-C₆ alkenyl and C₂-C₆ alkynyl is independently substituted with hydrogen, halogen, —CN, alkyl, haloalkyl, alkoxyalkyl, dialkylaminoalkyl, and heterocyclic alkyl;
R₅ is chosen from halogen, C₁-C₄ alkyl, C₂-alkynyl, CF₃, deuterated C₁-C₄ alkyl, amino group, —CN, hydroxyl, C₁-C₄alkoxy, and cycloalkyl;
R₆ is chosen from halogen, C₁-C₃ alkyl, C₁-C₃haloalkyl, dialkylamino group, amino group, —CN, hydroxyl, C₁-C₃alkoxy, cycloalkyl, heterocycle, heterocycloalkyl, and heteroaryl;
L is a bond, O, S, NH, or alkylamino;
Q is a 4-12 membered heteromonocyclic group, bridged or spiro heterocyclic group, wherein each of the heteromonocyclic group, bridged or spiro heterocyclic group is independently substituted with 0, 1, 2, 3, or 4 C₁-C₄alkyl, and wherein the C₁-C₄alkyl is further substituted with hydrogen, halogen, —CN, ester, and carboxamide;
with the proviso that the compound of Formula (1) is not
2-((2S)-1-acryloyl-4-(2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((2S)-1-acryloyl-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((S)-1-acryloyl-4-((S)-2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((S)-1-acryloyl-4-((R)-2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((S)-1-acryloyl-4-((R)-7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((S)-1-acryloyl-4-((S)-7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((S)-1-acryloyl-4-((S)-7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((S)-1-acryloyl-4-((R)-7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-(2-fluoroacryloyl)-4-(7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((S)-1-(2-fluoroacryloyl)-4-((S)-7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((S)-1-(2-fluoroacryloyl)-4-((R)-7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile;
2-((S)-4-((S)-7-(8-chloronaphthalen-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile;
2-((S)-4-((R)-7-(8-chloronaphthalen-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile;
2-((2S)-1-((E)-4-(dimethylamino)but-2-enoyl)-4-(7-(8-methylnaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile; or
2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-1-((E)-4-(dimethylamino)but-2-enoyl)piperazin-2-yl)acetonitrile.

66. The compound according to claim 65, wherein R₁ is chosen from

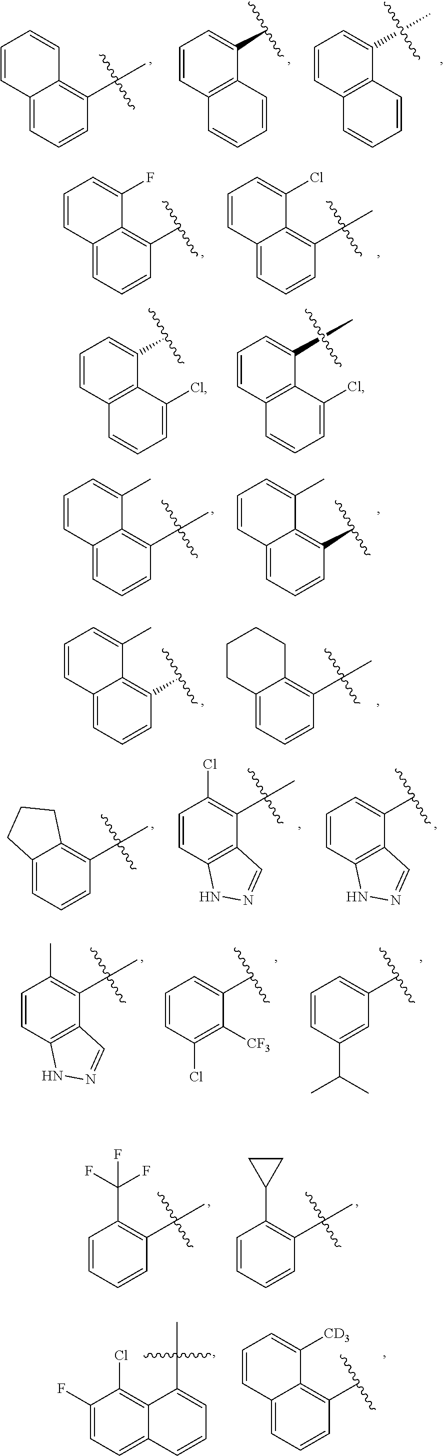

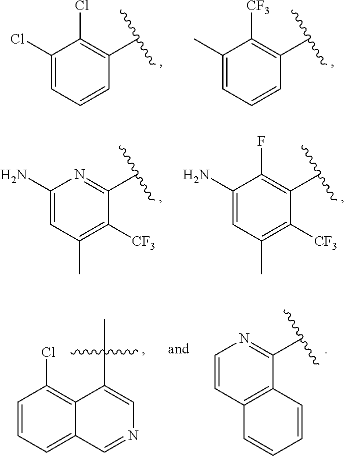

67. The compound according to claim 65, wherein R₁ is chosen from

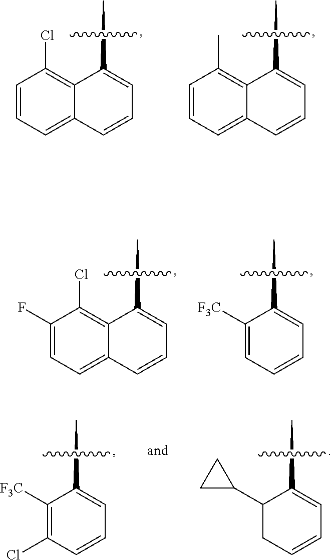

68. The compound according to claim 65, wherein R₅ is chosen from F, Cl, Br, I, —CF₃, —CD₃, —NH₂, —CH₃,

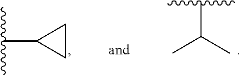

69. The compound according to claim 65, wherein R₂ is chosen from hydrogen, C₁-C₃ alkyl, and cycloalkyl.

70. The compound according to claim 69, wherein R₂ is chosen from hydrogen, —CH₃,

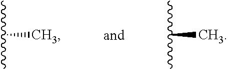

71. The compound according to claim 65, wherein R₃ is chosen from

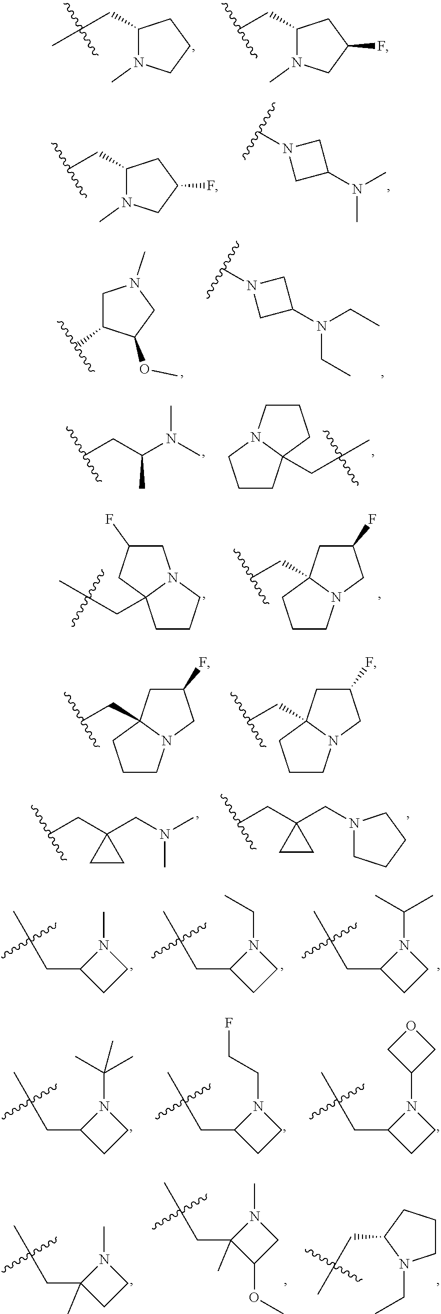

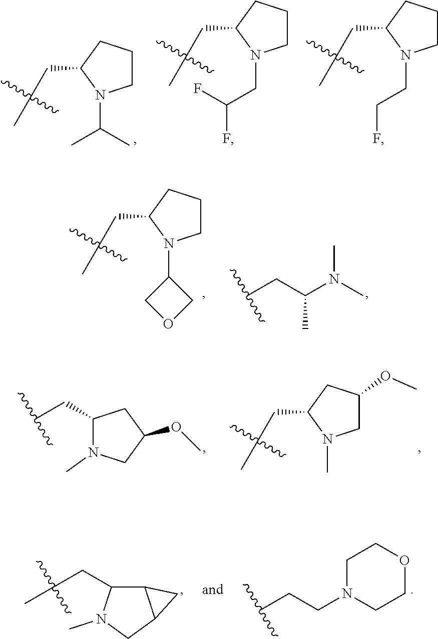

72. The compound according to claim 65, wherein R₃ is chosen from

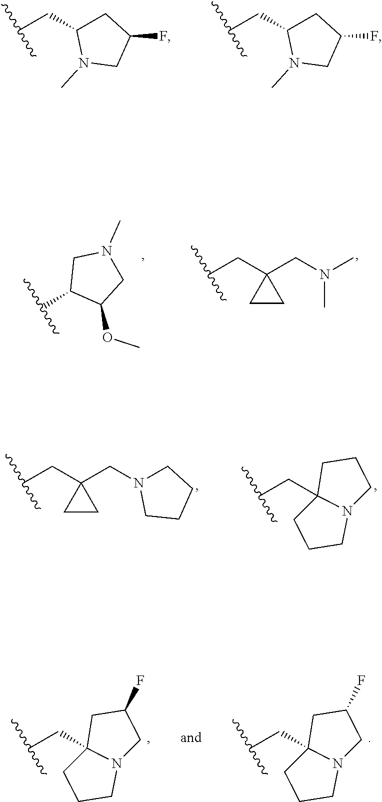

73. The compound according to claim 65, wherein R₆ is chosen from halogen, C₁-C₂ alkyl, C₁-C₂haloalkyl, C₁-C₂alkoxy, and dialkylamino group.

74. The compound according to claim 73, wherein R₆ is chosen from —CH₃,

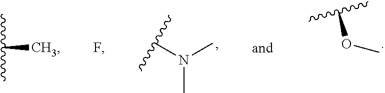

75. The compound according to claim 65, wherein R₄ is chosen from

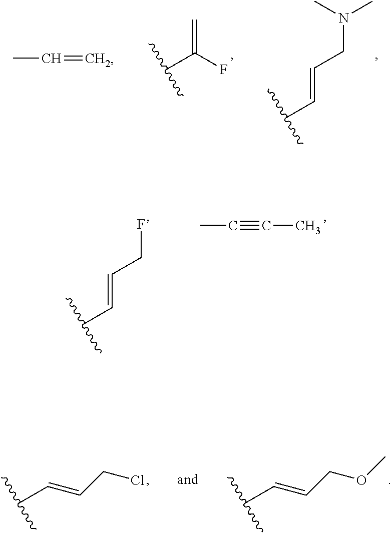

76. The compound according to claim 65, wherein L is a bond or O.

77. The compound according to claim 76, wherein L is O.

78. The compound according to claim 65, wherein Q is a 4-12 membered heteromonocyclic group, bridged or spiro heterocyclic group, wherein each of the heteromonocyclic group, bridged or spiro heterocyclic group is independently substituted with 0, 1, or 2 C₁-C₄alky.

79. The compound according to claim 78, wherein the C₁-C₄alkyl is further substituted with halogen, —CN, and carboxamide.

80. The compound according to claim 78, wherein Q is chosen from

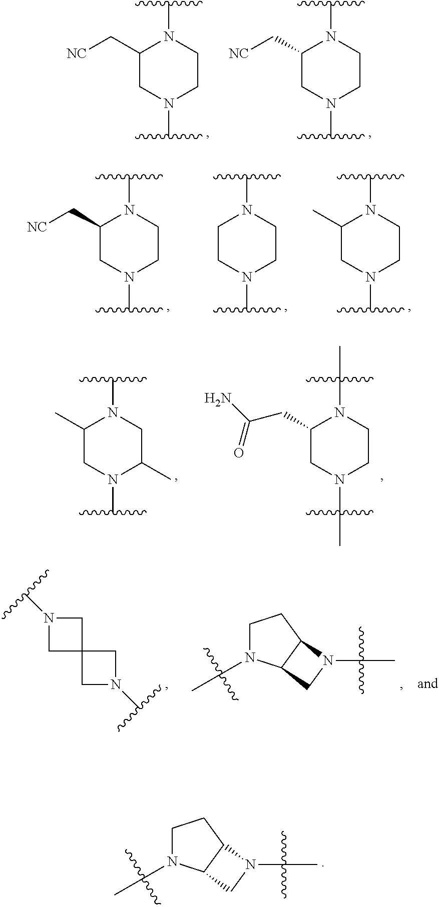

81. The compound according to claim 65, wherein the compound is chosen from:
2-((2S)-1-(2-fluoroacryloyl)-4-(2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((2S)-1-acryloyl-4-(2-(((2S,4R)-4-fluoro-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((S)-1-acryloyl-4-((S)-2-(((2S,4S)-4-fluoro-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((S)-1-acryloyl-4-((R)-2-(((2S,4S)-4-fluoro-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((2S)-1-acryloyl-4-(7-(8-chloronaphthalen-1-yl)-2-(((2S,4S)-4-fluoro-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(2-(((2S,4S)-4-fluoro-1-methylpyrrolidin-2-yl)methoxy)-7-(8-methylnaphthalen-1-yl)-7,8-dihydro-5H-pyrano[4,3-d]pyrimidin-4-yl)piperazin-2-yl)acetonitrile;
2-((2S)-4-(7-(8-chloronaphthalen-1-yl)-2-(((2S,4S)-4-fluoro-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile;
2-((2S)-4-(2-(((2S,4S)-4-fluoro-1-methylpyrrolidin-2-yl)methoxy)-7-(8-methylnaphthalen-1-yl)-7,8-dihydro-5H-pyrano[4,3-d]pyrimidin-4-yl)-1-(2-fluoroacryloyl)piperazin-2-yl)acetonitrile;
2-((2S)-1-acryloyl-4-(7-(8-chloronaphthalen-1-yl)-2-(3-(dimethylamino)azetidin-1-yl)-7,8-dihydro-5H-pyrano[4,3-d]pyrimidin-4-yl)piperazin-2-yl)acetonitrile;
2-((2S)-1-acryloyl-4-(7-(8-chloronaphthalen-1-yl)-2-(((3R,4R)-4-methoxy-1-methylpyrrolidin-3-yl)oxy)-7,8-dihydro-5H-pyrano[4,3-d]pyrimidin-4-yl)piperazin-2-yl)acetonitrile;
2-((2S)-1-acryloyl-4-(7-(8-chloronaphthalen-1-yl)-2-(3-(diethylamino)azetidin-1-yl)-7,8-dihydro-5H-pyrano[4,3-d]pyrimidin-4-yl)piperazin-2-yl)acetonitrile;
2-((2S)-1-acryloyl-4-(2-((S)-2-(dimethylamino)propoxy)-7-(naphthalen-1-yl)-7,8-dihydro-5H-pyrano[4,3-d]pyrimidin-4-yl)piperazin-2-yl)acetonitrile;
2-((2S)-1-acryloyl-4-(2-((R)-2-(dimethylamino)propoxy)-7-(naphthalen-1-yl)-7,8-dihydro-5H-pyrano[4,3-d]pyrimidin-4-yl)piperazin-2-yl)acetonitrile;
2-((2S)-1-acryloyl-4-(2-(((S)-1-methylpyrrolidin-2-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile;
2-((2S)-1-acryloyl-4-(7-(2,3-dihydro-1H-inden-4-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(5-chloro-1H-indazol-4-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-4-(7-(1H-indazol-4-yl)-2-(((S)-1-methylpyrrolidin-2-yl)methoxy)-7,8-dihydro-5H-pyrano[4,3-d]pyrimidin-4-yl)-1-acryloylpiperazin-2-yl)acetonitrile;
2-((2S)-1-acryloyl-4-(7-(5-methyl-1H-indazol-4-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(3-chloro-2-(trifluoromethyl)phenyl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(3-isopropylphenyl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(8-fluoronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(8-(methyl-d3)naphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(2,3-dichlorophenyl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(3-methyl-2-(trifluoromethyl)phenyl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(6-amino-4-methyl-3-(trifluoromethyl)pyridin-2-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(3-amino-2-fluoro-5-methyl-6-(trifluoromethyl)phenyl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(7-(isoquinolin-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-acryloyl-4-(5-methyl-2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((S)-1-acryloyl-4-((5S,7S)-5-methyl-2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-1-((E)-4-fluorobut-2-enoyl)piperazin-2-yl)acetonitrile;
2-((2S)-1-(but-2-ynoyl)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile;
2-((2S)-1-((E)-4-chlorobut-2-enoyl)-4-(2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((2S)-1-((E)-4-methoxybut-2-enoyl)-4-(2-(((S)-1-methylpyrrolidin-2-yl)methoxy)-7-(naphthalen-1-yl)-7,8-dihydro-5H-pyrano[4,3-d]pyrimidin-4-yl)piperazin-2-yl)acetonitrile;
2-((2S)-4-(7-(8-chloronaphthalen-1-yl)-2-(((2S,4R)-4-methoxy-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile;
2-((2S)-4-(7-(8-chloronaphthalen-1-yl)-2-(2-morpholinoethoxy)-7,8-dihydro-5H-pyrano[4,3-d]pyrimidin-4-yl)-1-(2-fluoroacryloyl)piperazin-2-yl)acetonitrile;
2-((2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile;
2-((S)-4-((R)-7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile;
2-((S)-4-((S)-7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile;
2-((2S)-1-(2-fluoroacryloyl)-4-(7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile;
2-((S)-1-(2-fluoroacryloyl)-4-((R)-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile;
2-((S)-1-(2-fluoroacryloyl)-4-((S)-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile;
2-((2S)-4-(7-(8-chloronaphthalen-1-yl)-5-methyl-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile;
2-((S)-1-(2-fluoroacryloyl)-4-((5R,7R)-5-methyl-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile;
2-((S)-1-(2-fluoroacryloyl)-4-((5S,7S)-5-methyl-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile;
2-((2S)-4-(7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile;
2-((S)-4-((R)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile;
2-((S)-4-((S)-7-(8-chloronaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile;
2-((S)-1-(2-fluoroacryloyl)-4-((5R,7R)-5-methyl-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile;
2-((S)-1-(2-fluoroacryloyl)-4-((5S,7S)-5-methyl-7-(8-methylnaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile;
2-((2S)-1-(2-fluoroacryloyl)-4-(2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile;
2-((S)-1-(2-fluoroacryloyl)-4-((R)-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile;
2-((S)-1-(2-fluoroacryloyl)-4-((S)-2-((tetrahydro-1H-pyrrolizin-7a(5H)-yl)methoxy)-7-(5,6,7,8-tetrahydronaphthalen-1-yl)-7,8-dihydro-5H-pyrano[4,3-d]pyrimidin-4-yl)piperazin-2-yl)acetonitrile;
2-((2S)-4-(7-(8-chloronaphthalen-1-yl)-2-((1-((dimethylamino)methyl)cyclopropyl)methoxy)-7,8-dihydro-5H-pyrano[4,3-d]pyrimidin-4-yl)-1-(2-fluoroacryloyl)piperazin-2-yl)acetonitrile;
2-((2S)-4-(7-(8-chloronaphthalen-1-yl)-2-((1-(pyrrolidin-1-ylmethyl)cyclopropyl)methoxy)-7,8-dihydro-5H-pyrano[4,3-d]pyrimidin-4-yl)-1-(2-fluoroacryloyl)piperazin-2-yl)acetonitrile;
2-((S)-4-((R)-7-(8-chloronaphthalen-1-yl)-2-((1-(pyrrolidin-1-ylmethyl)cyclopropyl)methoxy)-7,8-dihydro-5H-pyrano[4,3-d]pyrimidin-4-yl)-1-(2-fluoroacryloyl)piperazin-2-yl)acetonitrile;
2-((S)-4-((S)-7-(8-chloronaphthalen-1-yl)-2-((1-(pyrrolidin-1-ylmethyl)cyclopropyl)methoxy)-7,8-dihydro-5H-pyrano[4,3-d]pyrimidin-4-yl)-1-(2-fluoroacryloyl)piperazin-2-yl)acetonitrile;
2-((S)-1-(2-fluoroacryloyl)-4-((S)-2-((tetrahydro-1H-pyrrolizin-7a(5H)-yl)methoxy)-7-(2-(trifluoromethyl)phenyl)-7,8-dihydro-5H-pyrano[4,3-d]pyrimidin-4-yl)piperazin-2-yl)acetonitrile;
2-((S)-1-(2-fluoroacryloyl)-4-((S)-2-(((2R,7aS)-2-fluorotetrahydro-1H-pyrrolizin-7a(5H)-yl)methoxy)-7-(2-(trifluoromethyl)phenyl)-7,8-dihydro-5H-pyrano[4,3-d]pyrimidin-4-yl)piperazin-2-yl)acetonitrile;
2-((S)-1-(2-fluoroacryloyl)-4-((S)-7-(3-methyl-2-(trifluoromethyl)phenyl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile;
2-((S)-1-(2-fluoroacryloyl)-4-((S)-2-(((2R,7aS)-2-fluorotetrahydro-1H-pyrrolizin-7a(5H)-yl)methoxy)-7-(3-methyl-2-(trifluoromethyl)phenyl)-7,8-dihydro-5H-pyrano[4,3-d]pyrimidin-4-yl)piperazin-2-yl)acetonitrile;
2-((S)-4-((S)-7-(3-chloro-2-(trifluoromethyl)phenyl)-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile;
2-((S)-4-((S)-7-(3-chloro-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile;
2-((S)-4-((S)-7-(2-cyclopropylphenyl)-2-((tetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile; and
2-((S)-4-((S)-7-(2-cyclopropylphenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-(2-fluoroacryloyl)piperazin-2-yl)acetonitrile.

82. A pharmaceutical composition comprising the compound according to claim 65 and at least one additional component selected from pharmaceutically acceptable carriers, pharmaceutically acceptable vehicles, and pharmaceutically acceptable excipients.

83. The pharmaceutical composition according to 82, wherein the compound is present in a therapeutically effective amount.

84. A method of treating cancer in a subject in need thereof, comprising administering to the subject an effective amount of the compound according to claim 65, wherein the cancer is selected from breast cancer, lung cancer, pancreatic cancer, colorectal cancer, gall bladder cancer, thyroid cancer, bile duct cancer, ovarian cancer, endometrial cancer, prostate cancer, and esophageal cancer.

85. A method of treating cancer in a subject in need thereof, comprising administering to the subject an effective amount of the pharmaceutical composition according to claim 82, wherein the cancer is selected from breast cancer, lung cancer, pancreatic cancer, colorectal cancer, gall bladder cancer, thyroid cancer, bile duct cancer, ovarian cancer, endometrial cancer, prostate cancer, and esophageal cancer.

86. A method of inhibiting KRAS G12C in a cell, comprising contacting the cell with the compound of claim 65.

87. The method according to claim 84, further comprising administering to the subject in combination with an anti-cancer agent.

88. The compound of Formula (1) according to claim 65, wherein the compound is a compound chosen from: [chemical structure]
